(19) United States
(12) Patent Application Publication
(10) Pub. No.: US 2023/0104183 A1
(43) Pub. Date: Apr. 6, 2023

(54) COMPOUND FOR ORGANIC ELECTRICAL ELEMENT, ORGANIC ELECTRICAL ELEMENT USING SAME, AND ELECTRONIC DEVICE THEREOF

(71) Applicant: DUK SAN NEOLUX CO., LTD., Cheonan-si, Chungcheongnam-do (KR)

(72) Inventors: Dae Hwan OH, Cheonan-si, Chungcheongnam-do (KR); Jong Gwang PARK, Cheonan-si, Chungcheongnam-do (KR); Won Sam KIM, Cheonan-si, Chungcheongnam-do (KR); Sun Hee LEE, Cheonan-si, Chungcheongnam-do (KR); Soung Yun MUN, Cheonan-si, Chungcheongnam-do (KR); Chi Hyun PARK, Cheonan-si, Chungcheongnam-do (KR)

(73) Assignee: DUK SAN NEOLUX CO., LTD., Cheonan-si, Chungcheongnam-do (KR)

(21) Appl. No.: 17/758,382

(22) PCT Filed: Jan. 12, 2021
(86) PCT No.: PCT/KR2021/000417

§ 371 Date: Jul. 5, 2022

(30) Foreign Application Priority Data
Jan. 14, 2020 (KR) 10-2020-0004894

(51) Int. Cl. C07D 307/91; C07D 333/76; C07D 409/12; H01L 51/00; C07D 405/12; H01L 51/50

(52) U.S. Cl. CPC C07D 307/91; C07D 333/76; C07D 409/12; H01L 51/0059; H01L 51/0073; C07D 405/12; H01L 51/0067; H01L 51/0074; H01L 51/0064; H01L 51/504; C07B 2200/05

(57) ABSTRACT

Provided are a compound capable of improving the luminous efficiency, stability and lifespan of an organic electronic device employing the same, an organic electronic element employing the same, and an electronic device thereof.

### BACKGROUND

### Technical Field

The present invention relates to a compound for an organic electronic element, an organic electronic element using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function.

Lifespan and efficiency are the most problematic in organic electroluminescent device, and as displays become larger, these problems of efficiency and lifespan must be solved. Efficiency, lifespan, and driving voltage are related to each other, and when the efficiency is increased, the driving voltage is relatively decreased, and as the driving voltage is decreased, crystallization of the organic material due to Joule heating generated during driving decreases, and as a result, the lifespan tends to increase.

However, the efficiency cannot be maximized simply by improving the organic material layer. This is because, when the energy level and T1 value between each organic material layer, and the intrinsic properties of the material (mobility, interfacial properties, etc.) are optimally combined, a long lifespan and high efficiency can be achieved at the same time.

Also, in order to solve the problem of light emission in the hole transport layer in recent organic electroluminescent devices, an emitting-auxiliary layer must exist between the hole transport layer and the emitting layer, and it is time to develop different emitting-auxiliary layers according to each emitting layer (R, G, B).

In general, electrons are transferred from the electron transport layer to the emitting layer, and holes are transferred from the hole transport layer to the emitting layer, and excitons are generated by recombination.

However, since the material used for the hole transport layer should have a low HOMO value, most have a low T1 value. As a result, excitons generated in the emitting layer are transferred to the hole transport layer, resulting in charge unbalance in the emitting layer to emit light at the hole transport layer interface.

When light is emitted at the hole transport layer interface, the color purity and efficiency of the organic electronic element are lowered, and the lifespan is shortened. Therefore, it is urgently required to develop an emitting-auxiliary layer having a high T1 value and having a HOMO level between the HOMO energy level of the hole transport layer and the HOMO energy level of the emitting layer.

Furthermore, it is necessary to develop a hole injection layer material that delays the penetration and diffusion of metal oxides from the anode electrode (ITO) into the organic layer, which is one of the causes of shortening the lifespan of organic electronic element, and that has stable characteristics, that is, a high glass transition temperature, even against Joule heating generated during device driving. The low glass transition temperature of the hole transport layer material has a characteristic of lowering the uniformity of the thin film surface during device driving, which is reported to have a significant effect on device lifespan. Moreover, OLED devices are mainly formed by a deposition method, and it is necessary to develop a material that can withstand a long time during deposition, that is, a material with strong heat resistance.

In other words, in order to fully exhibit the excellent characteristics of an organic electronic element, it should be preceded that the material constituting the organic material layer in the device, such as a hole injection material, a hole transport material, a light emitting material, an electron transport material, an electron injection material, emitting auxiliary layer material, etc., is supported by a stable and efficient material, but the development of a stable and efficient organic material layer material for an organic electronic device has not yet been sufficiently made. Therefore, the development of new materials is continuously required.

As a reference prior art document, KR1020130076842 A was used.

### DETAILED DESCRIPTION OF THE INVENTION

### Summary

In order to solve the problems of the above-mentioned background art, the present invention has revealed a compound having a novel structure, and when this compound is applied to an organic electronic element, it has been found that the luminous efficiency, stability and lifespan of the device can be significantly improved.

Accordingly, an object of the present invention is to provide a novel compound, an organic electronic element using the same, and an electronic device thereof.

### Technical Solution

The present invention provides a compound represented by Formula 1.

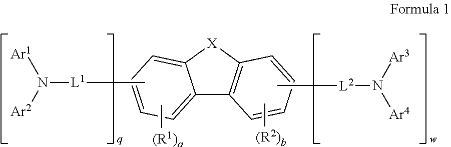

In another aspect, the present invention provides an organic electronic element comprising the compound represented by Formula 1 and an electronic device thereof.

### Effects of the Invention

By using the compound according to the present invention, high luminous efficiency, low driving voltage and high heat resistance of the device can be achieved, and color purity and lifespan of the device can be greatly improved.

[BRIEF DESCRIPTION OF THE DRAWINGS]

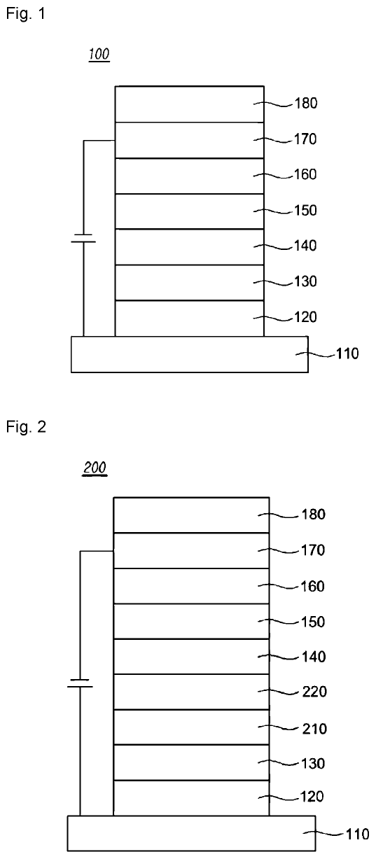

FIG. 1 to FIG.3 are exemplary views of an organic electroluminescent
device according to the present invention.
FIG. 4 shows a Formula according to an aspect of the present invention.

| | | |
|---|---|---|
| 100, 200, 300: organic electronic element | 110 : | the first electrode |
| 120 : hole injection layer | 130 : | hole transport layer |
| 140 : emitting layer | 150 : | electron transport layer |
| 160 : electron injection layer | 170 : | second electrode |
| 180 : light efficiency enhancing Layer | 210 : | buffer layer |
| 220 : emitting auxiliary layer | 320 : | first hole injection layer |
| 330 : first hole transport layer | 340 : | first emitting layer |
| 350 : first electron transport layer | 360 : | first charge generation layer |
| 361 : second charge generation layer | 420 : | second hole injection layer |
| 430 : second hole transport layer | 440 : | second emitting layer |
| 450 : second electron transport layer | CGL | : charge generation layer |
| ST1 : first stack | ST2 | : second stack |

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear. In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected “, ” coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an oxygen radical attached to an alkyl group, but is not limited thereto, and has 1 to 60 carbon atoms.

Unless otherwise stated, the term “aryloxyl group” or “aryloxy group”, as used herein, means an oxygen radical attached to an aryl group, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or an arylene group means a single ring or multiple ring aromatic, and includes an aromatic ring formed by an adjacent substituent joining or participating in a reaction.

For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

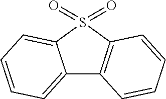 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

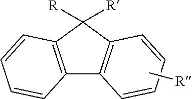

The term “Spiro compound”, as used herein, has a ‘Spiro union’, and a Spiro union means a connection in which two rings share only one atom. At this time, atoms shared in the two rings are called ‘spiro atoms’, and these compounds are called ‘monospiro-’, ‘di-spiro ’ and ‘tri-spiro’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless there is an explicit explanation, the formula used in the present invention is the same as the definition of the substituent by the exponent definition of the following formula.

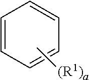

Here, when a is an integer of zero, the substituent R¹ is absent, when a is an integer of 1, the sole substituent R¹ is linked to any one of the carbon constituting the benzene ring, when a is an integer of 2 or 3, each is bonded as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

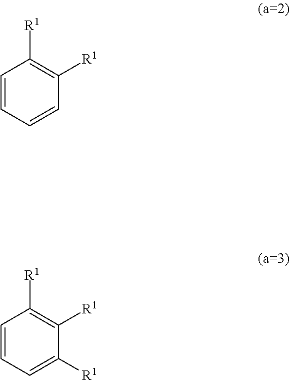

Hereinafter, a compound according to an aspect of the present invention and an organic electronic element including the same will be described.

The present invention provides a compound represented by Formula 1.

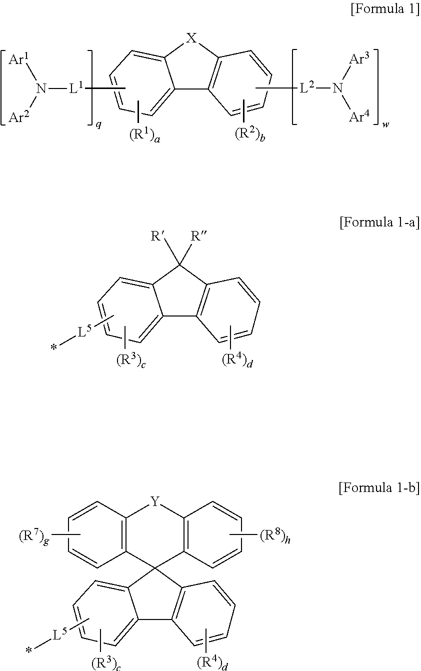

{wherein, each symbol may be defined as follows.

X is O or S,

R¹ and R² are each independently selected from the group consisting of a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring; C₁-C₆₀ alkyl group; a C₂-C₆₀ alkenyl group; a C₂-C₆₀ alkynyl group; a C₁-C₆₀ alkoxyl group; a C₆-C₆₀ aryloxy group; or, in case a and b are 2 or more, R¹ and R² are each in plural being the same or different.

Wherein in case R¹ and R² are an aryl group, it may be preferably a C₆-C₃₀ aryl group, and more preferably a C₆-C₂₅ aryl group, for example, it may be phenylene, biphenyl, naphthalene, terphenyl, etc.

In case R¹ and R² are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case R¹ and R² are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

In case R¹ and R² are an alkyl group, it may be preferably a C₁-C₃₀ alkyl group, and more preferably a C₁-C₂₄ alkyl group.

In case R¹ and R² are an alkenyl group, they may be preferably an C₂˜C₃₀ alkenyl group, more preferably an C₂˜-C₂₄ alkenyl group.

In case R¹ and R² are an alkynyl group, they may be preferably an C₂˜C₃₀ alkynyl group, more preferably an C₂˜C₂₄ alkynyl group.

In case R¹ and R² are an alkoxyl group, it may be preferably a C₁-C₃₀ alkoxyl group, more preferably an C₁˜C₂₄ alkoxyl group.

In case R¹ and R² are an aryloxy group, it may be preferably a C₆-C₃₀ aryloxy group, more preferably an C₆˜C₂₄ aryloxy group.

Wherein L¹ and L² are each independently selected from the group consisting of a single bond, a C₆-C₆₀ arylene group, a C₂-C₆₀ heteroarylene group including at least one heteroatom of O, N, S, Si or P; or in case q and w are 2 or more, L¹ and L² are each in plural being the same or different.

In case L¹ and L² are an arylene group, it may be preferably a C₆-C₃₀ arylene group, more preferably a C₆-C₂₄ arylene group, for example, phenylene, biphenyl, naphthalene, terphenyl, etc.

In case L¹ and L² are an heteroarylene group, it may be preferably a C₂-C₃₀ heteroarylene group, more preferably a C₂-C₂₄ heteroarylene group,

Wherein Ar¹, Ar², Ar³ and Ar⁴ are each independently selected from the group consisting of a C₆-C₆₀ aryl group; a substituent represented by Formula 1-a; and a substituent represented by Formula 1-b, however, at least one pair of Ar¹ and Ar², and Ar³ and Ar⁴ is a substituent represented by Formula 1-a.

In case Ar¹, Ar², Ar³ and Ar⁴ are an aryl group, they may be preferably a C₆-C₃₀ aryl group, most preferably a C₆-C₂₅ aryl group, for example, phenylene, biphenyl, naphthalene, terphenyl, and the like.

a and b are each independently an integer of 0 to 4, q and w are each independently an integer of 0 to 2, with the proviso that q+w is 2 or more,

In Formulas 1-a to 1-b, each symbol may be defined as follows.

Y is NR^{a}, O or S,

R^{a} is selected from the group consisting of a C₆-C₆₀ aryl group; a C₂-C₆₀ heteroaryl group including at least one heteroatom of O, N, S, Si or a C₂-C₆₀ heterocyclic group including at least one heteroatom of O, N, S, Si or and —L′—N(R^{c})(R^{d});

In case R^{a} is an aryl group, it may be preferably a C₆-C₃₀ aryl group, and more preferably a C₆-C₂₅ aryl group, for example, it may be phenylene, biphenyl, naphthalene, terphenyl, etc.

In case R^{a} is a heteroaryl group, it may be preferably a C₂-C₃₀ heteroaryl group, and more preferably a C₂-C₂₄ heteroaryl group,

In case R^{a} is a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

L′ is selected from the group consisting of a C₆-C₆₀ arylene group; a fluorenylene group; a fused ring group of a C₃-C₆₀ aliphatic ring and a C₆-C₆₀ aromatic ring; and a C₂-C₆₀ heterocyclic group including at least one heteroatom of O, N, S, Si or P;

In case L′ is an arylene group, it may be preferably a C₆-C₃₀ arylene group, more preferably a C₆-C₂₄ arylene group, for example, phenylene, biphenyl, naphthalene, terphenyl, etc.

In case L′ is a heterocyclic group, it may be preferably a C₂˜C₃₀ heterocyclic group, and more preferably a C₂˜C₂₄ heterocyclic group, for example, pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case L′ is fused ring groups, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

R^{c} and R^{d} are each independently selected from the group consisting of a C₆-C₆₀ aryl group; a fluorenyl group; a fused ring group of a C₃-C₆₀ aliphatic ring and a C₆-C₆₀ aromatic ring; a C₂-C₆₀ heterocyclic group including at least one heteroatom of O, N, S, Si or P;

In case R^{c} and R^{d} are an aryl group, it may be preferably a C₆-C₃₀ aryl group, and more preferably a C₆-C₂₅ aryl group, for example, it may be phenylene, biphenyl, naphthalene, terphenyl, etc.

In case R^{c} and R^{d} are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

In case R^{c} and R^{d} are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

R³, R⁴, R⁷ and R⁸ are each independently selected from the group consisting of a C₆-C₆₀ aryl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₆₀ alkyl group; an C₂-C₆₀ alkenyl group; a C₂-C₆₀ alkynyl group; a C₁-C₆₀ alkoxyl group; a C₆-C₆₀ aryloxy group; or in case a, b, c and d are 2 or more, R³, R⁴, R⁷ and R⁸ are each in plural being the same or different.

Wherein in case R³, R⁴, R⁷ and R⁸ are an aryl group, it may be preferably a C₆-C₃₀ aryl group, and more preferably a C₆-C₂₅ aryl group, for example, it may be phenylene, biphenyl, naphthalene, terphenyl, etc.

In case R³, R⁴, R⁷ and R⁸ are a heterocyclic group, it may be preferably a C₂-C₃₀ heterocyclic group, and more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

In case R³, R⁴, R⁷ and R⁸ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

In case R³, R⁴, R⁷ and R⁸ are an alkyl group, it may be preferably a C₁-C₃₀ alkyl group, and more preferably a C₁-C₂₄ alkyl group.

In case R³, R⁴, R⁷ and R⁸ are an alkenyl group, it may be preferably an C₂˜C₃₀ alkenyl group, more preferably an C₂˜C₂₄ alkenyl group.

In case R³, R⁴, R⁷ and R⁸ are an alkynyl group, it may be preferably an C₂˜C₃₀ alkynyl group, more preferably an C₂˜C₂₄ alkynyl group.

In case R³, R⁴, R⁷ and R⁸ are an alkoxyl group, it may be preferably a C₁-C₃₀ alkoxyl group, more preferably an C₁˜C₂₄ alkoxyl group.

In case R³, R⁴, R⁷ and R⁸ are an aryloxy group, it may be preferably a C₆-C₃₀ aryloxy group, more preferably an C₆˜C₂₄ aryloxy group.

d, g and h are each independently an integer from 0 to 4, c is an integer from 0 to 3,

L⁵ and L₅ are each independently selected from the group consisting of a single bond; a C₆-C₆₀ arylene group; a C₂-C₆₀ heteroarylene group including at least one heteroatom of O, N, S, Si or P;

In case L⁵ and L₅ are an arylene group, it may be preferably a C₆-C₃₀ arylene group, and more preferably a C₆-C₂₄ arylene group,

In case L⁵ and L₅ are a heteroarylene group, it may be preferably a C₂-C₃₀ heteroarylene group, and more preferably a C₂-C₂₄ heteroarylene group,

R′ and R″ are independently selected from the group consisting of hydrogen; deuterium; halogen; cyano group; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heteroaryl group including at least one heteroatom of O, N, S, Si or P; a fused ring group of a C₃-C₆₀ aliphatic ring and a C₆-C₆₀ aromatic ring; wherein R′ and R″ are bonded to each other to form a spiro.

In case R′ and R″ are an alkyl group, it may be preferably a C₁-C₃₀ alkyl group, and more preferably a C₁₁-C₂₄ alkyl group.

In case R′ and R″ are an alkenyl group, they may be preferably an C₂˜C₃₀ alkenyl group, more preferably an C₂-C₂₄ alkenyl group.

In case R′ and R″ are an alkynyl group, they may be preferably an C₂˜C₃₀ alkynyl group, more preferably an C₂˜C₂₄ alkynyl group.

In case R′ and R″ are an alkoxyl group, it may be preferably a C₁-C₃₀ alkoxyl group, more preferably an C₁˜C₂₄ alkoxyl group.

In case R′ and R″ are an aryloxy group, it may be preferably a C₆-C₃₀ aryloxy group, more preferably an C₆˜C₂₄ aryloxy group.

In case R′ and R″ are an aryl group, it may be preferably a C₆-C₃₀ aryl group, more preferably an C₆˜C₂₅ aryl group, for example, it may be phenylene, biphenyl, naphthalene, terphenyl, etc.

In case R′ and R″ are a heteroaryl group, it may be preferably a C₂-C₃₀ heteroaryl group, more preferably an C₂˜C₂₄ heteroaryl group.

In case R′ and R″ are a fused ring group, it may be preferably a fused ring group of a C₃-C₃₀ aliphatic ring and a C₆-C₃₀ aromatic ring, more preferably a fused ring group of a C₃-C₂₄ aliphatic ring and a C₆-C₂₄ aromatic ring.

* indicates the position to be bonded,

wherein the aryl group, arylene group, heteroarylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group; and C₈-C₂₀ arylalkenyl group; and —L′—N(R^{c})(R^{d}); also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, Formula 1 includes a compound represented by Formula 2.

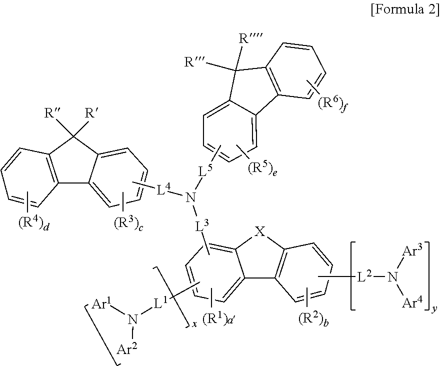

{Wherein X, R¹, R², R³, R⁴, L¹, L², L⁵, Ar¹, Ar², Ar³, Ar⁴, R′ and R″ are the same as defined above,

a′, c and e are each independently an integer of 0 to 3, b, d and f are each independently an integer of 0 to 4,

x is 0 or 1, y is an integer from 0 to 2, provided that x+y is 1 or more,

R⁵ and R⁶ are the same as the definitions of R¹ to R² above,

L³ and L⁴ are the same as the definitions of L² and L⁵ above,

R′″ and R″″ have the same definitions as R′ and R″ above.}

Also, Formula 1 is represented by Formulas 2-1 to 2-5

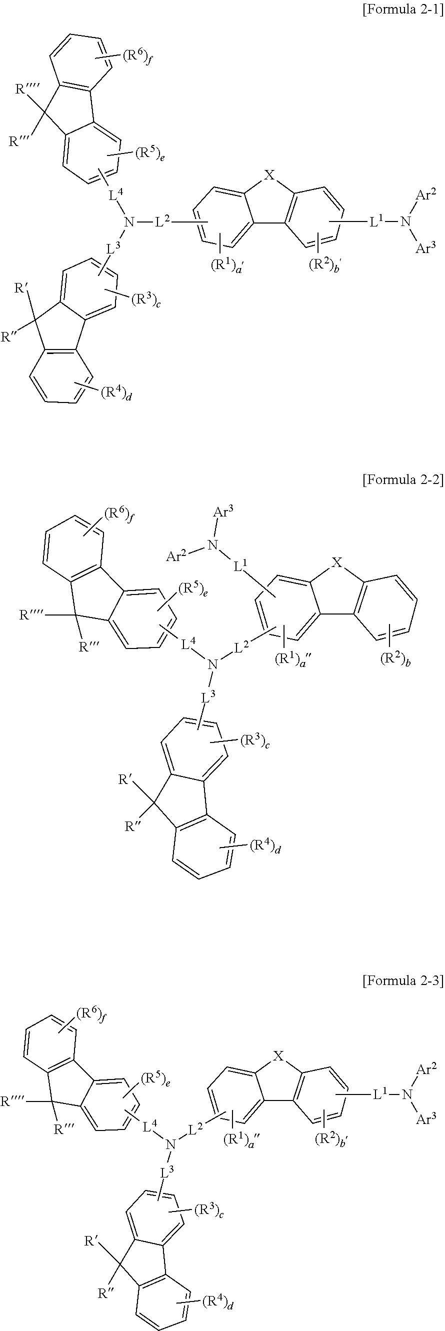

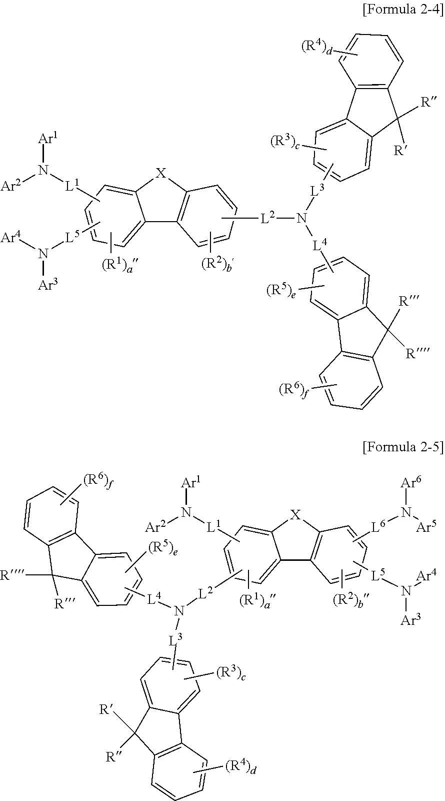

Wherein, X, R¹, R², R³, R⁴, R⁵, R⁶, L¹, L², L³, L⁴, L⁵, Ar¹, Ar², Ar³, Ar⁴, R′, R″, R′″, R″″, b, c, d, e and f are the same as defined above,

L⁶ is the same as the definition of L¹ to L⁵,

Ar⁵ and Ar⁶ are the same as the definitions of Ar¹ to Ar⁴,

a″ and b″ are independently integers from 0 to 2, and a′ and b′ are independently integers from 0 to 3.

Also, Formula 1 is represented by Formula 2-6 or Formula 2-7

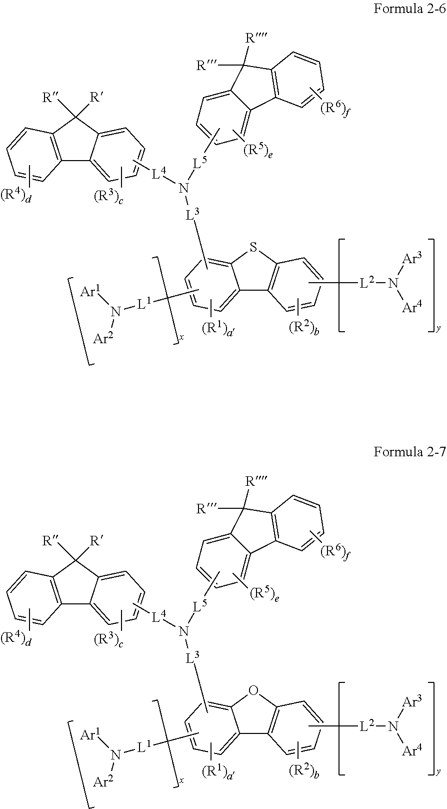

{Wherein R¹, R², R³, R⁴, R⁵, R⁶, L¹, L², L³, L⁴, L⁵, Ar¹, Ar², Ar³, Ar⁴, R′, R″, R′″, R″″, a′, b, c, d, e, f, x and y are the same as defined above.}

Also, Formula 1 is represented by Formulas 2-8 to 2-11.

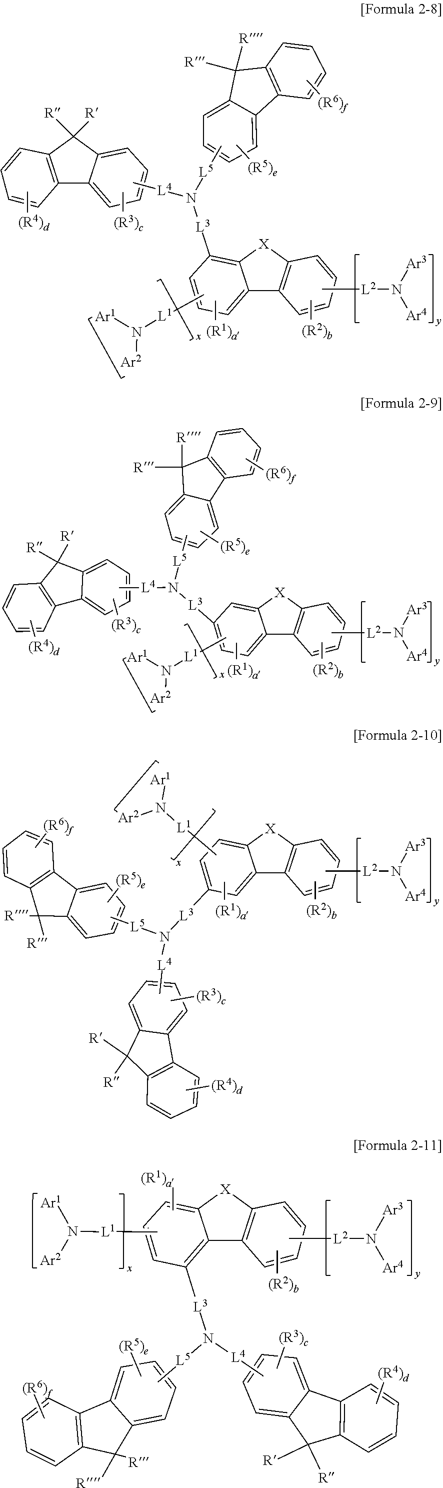

{Wherein X, R¹, R², R³, R⁴, R⁵, R⁶, L¹, L², L³, L⁴, L⁵, Ar¹, Ar², Ar³, Ar⁴, R′, R″, R′″, R″″, a′, b, c, d, e, f, x and y are the same as defined above.}

Also, Formula 1 is represented by Formulas 2-12 to 2-15

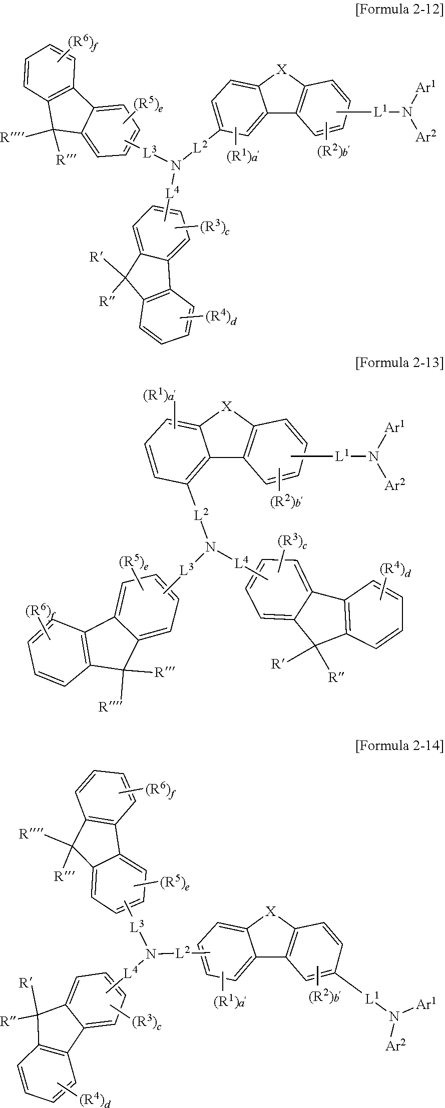

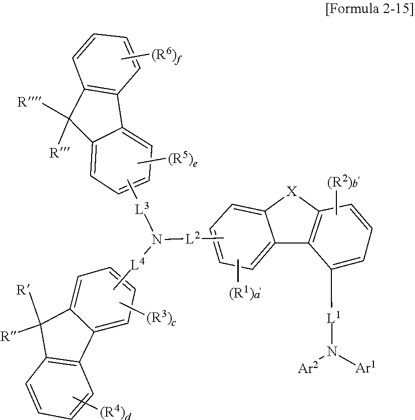

{Wherein X, R¹, R², R³, R⁴, R⁵, R⁶, L¹, L², L³, L⁴, Ar¹, Ar², R′, R″, R′″ and R″″ are the same as defined above,

a′, b′, c and e are independently integers from 0 to 3, and b and f are independently integers from 0 to 4.}

Also, Formula 1 is represented by Formulas 2-16 to 2-19

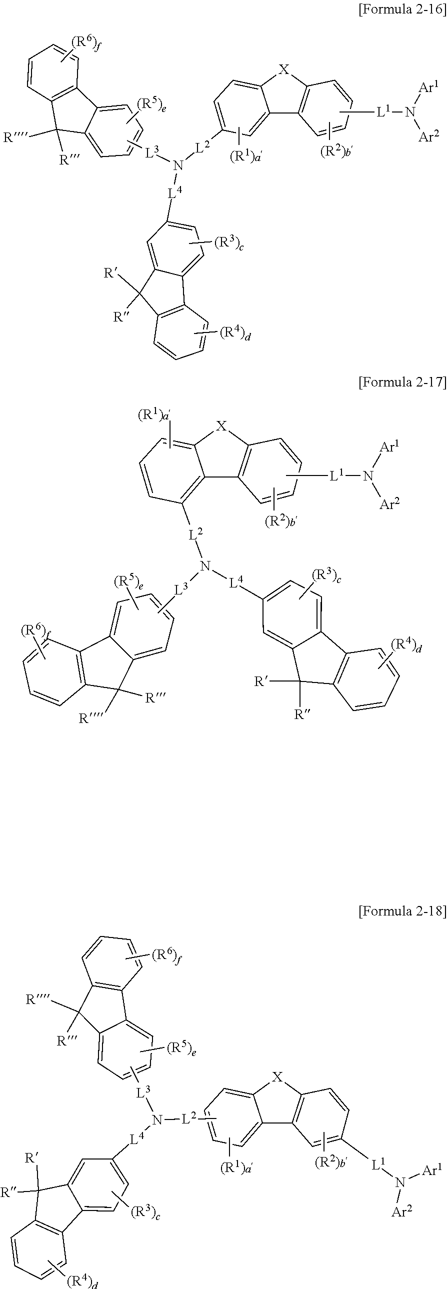

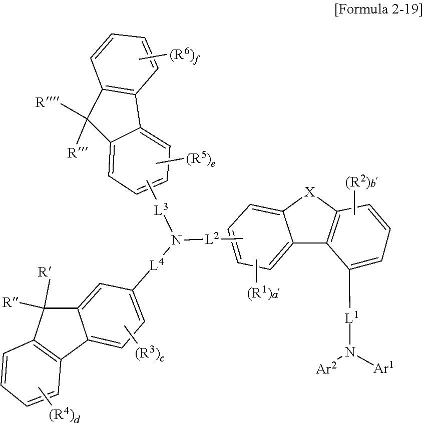

{Wherein X, R¹, R², R³, R⁴, R⁵, R⁶, L¹, L², L³, L⁴, Ar¹, Ar², R′, R″, R′″ and R″″ are the same as defined above,

a′, b′, c and e are independently integers from 0 to 3, and b and f are independently integers from 0 to 4.}

Also, Formula 1 is represented by Formula 2-20.

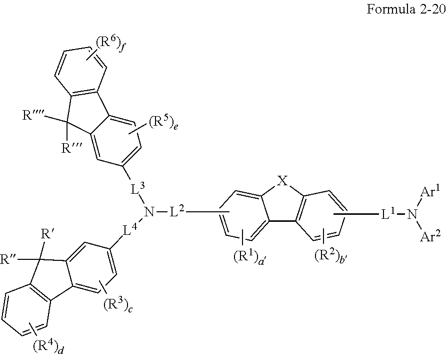

{Wherein X, R¹, R², R³, R⁴, R⁵, R⁶, L¹, L², L³, L⁴, Ar¹, Ar², R′, R″, R′″ and R″″ are the same as defined above,

a′, b′, c and e are independently integers from 0 to 3, and b and f are independently integers from 0 to 4.}

Specifically, the compound represented by Formula 1 may be any one of the following compounds P-1 to P-60, but is not limited thereto.

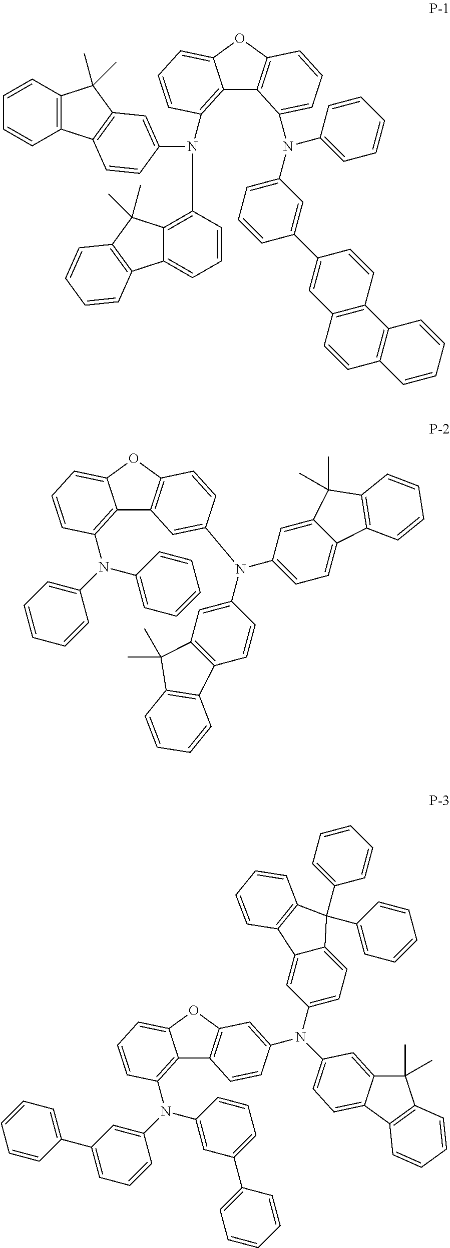 [SMILES: CC1(C)c2ccccc2-c2ccc(N(c3cccc4c3C(C)(C)c3ccccc3-4)c3cccc4oc5cccc(N(c6ccccc6)c6cccc(-c7ccc8c(ccc9ccccc98)c7)c6)c5c34)cc21 ; CC1(C)c2ccccc2-c2ccc(N(c3ccc4c(c3)C(C)(C)c3ccccc3-4)c3ccc4oc5cccc(N(c6ccccc6)c6ccccc6)c5c4c3)cc21 ; CC1(C)c2ccccc2-c2ccc(N(c3ccc4c(c3)-c3ccccc3C4(c3ccccc3)c3ccccc3)c3ccc4c(c3)oc3cccc(N(c5cccc(-c6ccccc6)c5)c5cccc(-c6ccccc6)c5)c34)cc21]

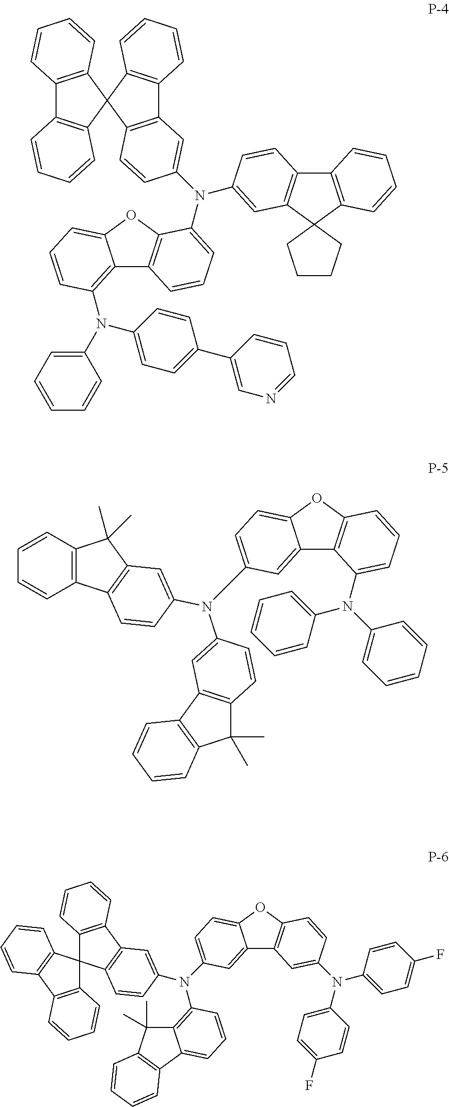 [SMILES: c1ccc(N(c2ccc(-c3cccnc3)cc2)c2cccc3oc4c(N(c5ccc6c(c5)-c5ccccc5C65c6ccccc6-c6ccccc65)c5ccc6c(c5)C5(CCCC5)c5ccccc5-6)cccc4c23)cc1 ; CC1(C)c2ccccc2-c2cc(N(c3ccc4c(c3)C(C)(C)c3ccccc3-4)c3ccc4oc5cccc(N(c6ccccc6)c6ccccc6)c5c4c3)ccc21 ; CC1(C)c2ccccc2-c2cccc(N(c3ccc4c(c3)-c3ccccc3C43c4ccccc4-c4ccccc43)c3ccc4oc5ccc(N(c6ccc(F)cc6)c6ccc(F)cc6)cc5c4c3)c21]

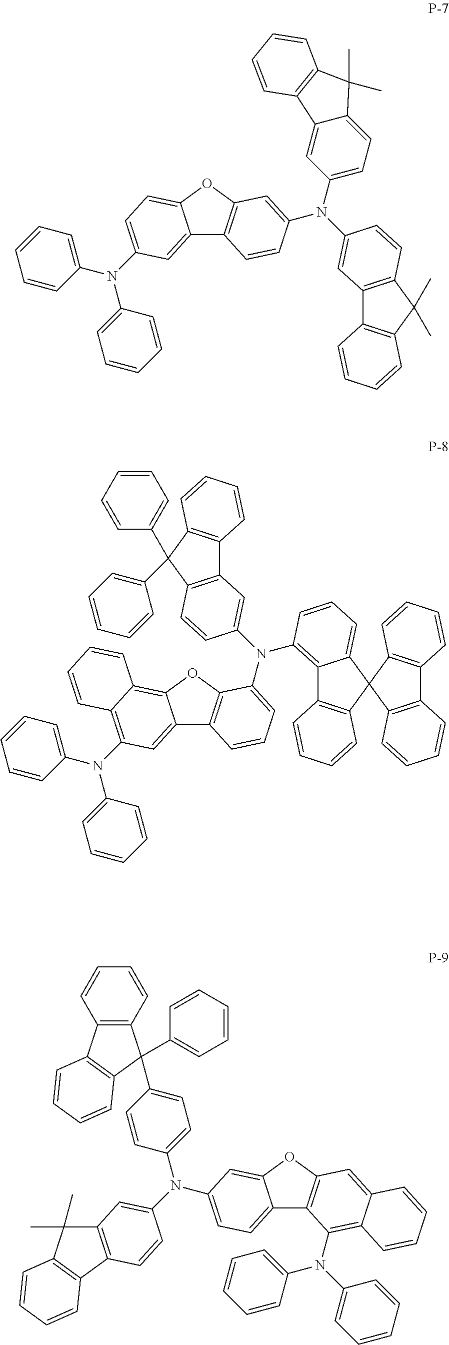 [SMILES: CC1(C)c2ccccc2-c2cc(N(c3ccc4c(c3)-c3ccccc3C4(C)C)c3ccc4c(c3)oc3ccc(N(c5ccccc5)c5ccccc5)cc34)ccc21 ; c1ccc(N(c2ccccc2)c2cc3c4cccc(N(c5ccc6c(c5)-c5ccccc5C6(c5ccccc5)c5ccccc5)c5cccc6c5-c5ccccc5C65c6ccccc6-c6ccccc65)c4oc3c3ccccc23)cc1 ; CC1(C)c2ccccc2-c2ccc(N(c3ccc(C4(c5ccccc5)c5ccccc5-c5ccccc54)cc3)c3ccc4c(c3)oc3cc5ccccc5c(N(c5ccccc5)c5ccccc5)c34)cc21]

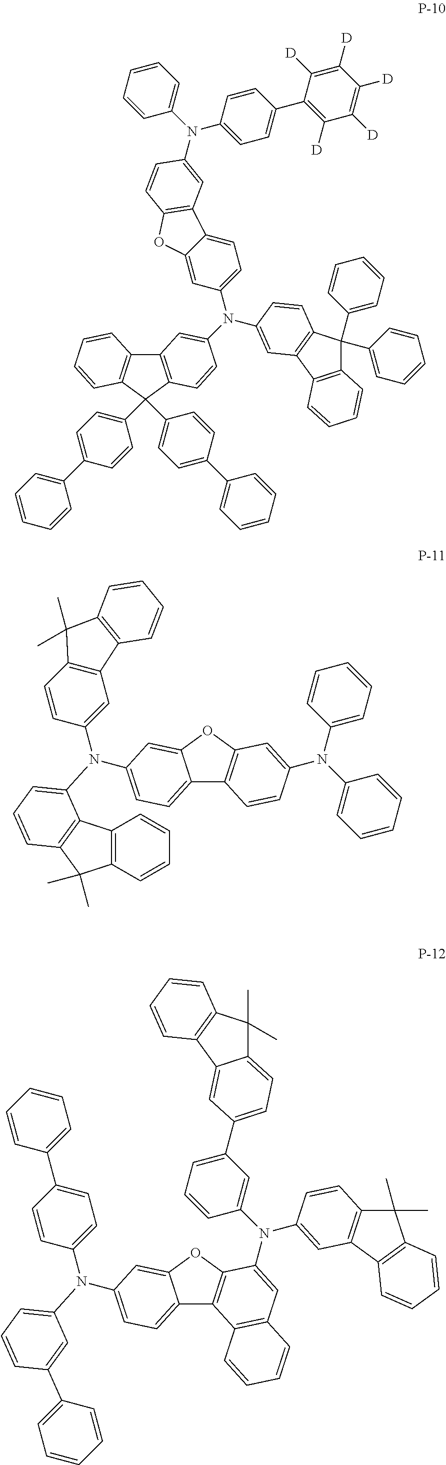 [SMILES: [2H]c1c([2H])c([2H])c(-c2ccc(N(c3ccccc3)c3ccc4oc5cc(N(c6ccc7c(c6)-c6ccccc6C7(c6ccccc6)c6ccccc6)c6ccc7c(c6)-c6ccccc6C7(c6ccc(-c7ccccc7)cc6)c6ccc(-c7ccccc7)cc6)ccc5c4c3)cc2)c([2H])c1[2H] ; CC1(C)c2ccccc2-c2cc(N(c3ccc4c(c3)oc3cc(N(c5ccccc5)c5ccccc5)ccc34)c3cccc4c3-c3ccccc3C4(C)C)ccc21 ; CC1(C)c2ccccc2-c2cc(-c3cccc(N(c4ccc5c(c4)-c4ccccc4C5(C)C)c4cc5ccccc5c5c4oc4cc(N(c6ccc(-c7ccccc7)cc6)c6cccc(-c7ccccc7)c6)ccc45)c3)ccc21]

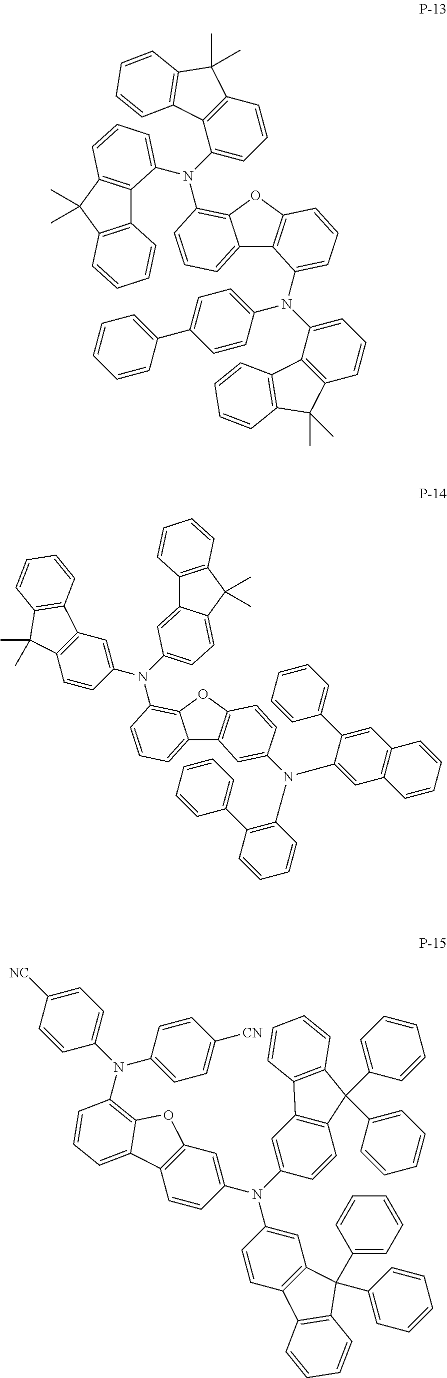 [SMILES: CC1(C)c2ccccc2-c2c(N(c3cccc4c3-c3ccccc3C4(C)C)c3cccc4c3oc3cccc(N(c5ccc(-c6ccccc6)cc5)c5cccc6c5-c5ccccc5C6(C)C)c34)cccc21 ; CC1(C)c2ccccc2-c2cc(N(c3ccc4c(c3)-c3ccccc3C4(C)C)c3cccc4c3oc3ccc(N(c5ccccc5-c5ccccc5)c5cc6ccccc6cc5-c5ccccc5)cc34)ccc21 ; N#Cc1ccc(N(c2ccc(C#N)cc2)c2cccc3c2oc2cc(N(c4ccc5c(c4)-c4ccccc4C5(c4ccccc4)c4ccccc4)c4ccc5c(c4)C(c4ccccc4)(c4ccccc4)c4ccccc4-5)ccc23)cc1]

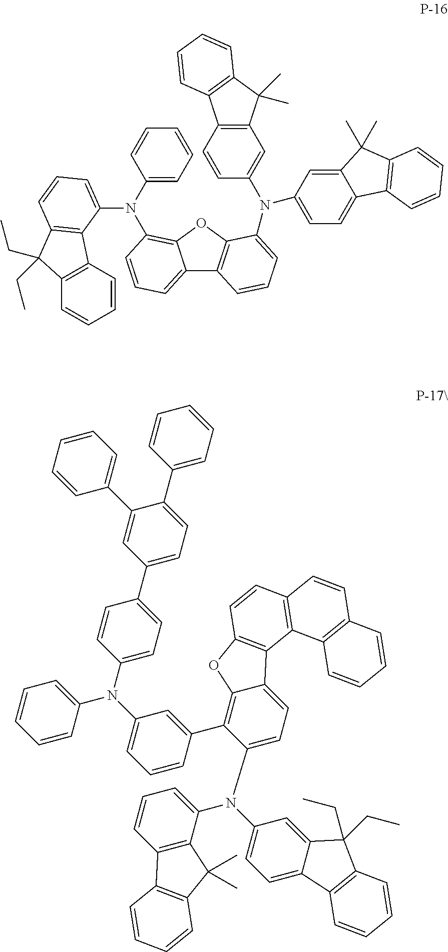 [SMILES: CCC1(CC)c2ccccc2-c2c(N(c3ccccc3)c3cccc4c3oc3c(N(c5ccc6c(c5)C(C)(C)c5ccccc5-6)c5ccc6c(c5)C(C)(C)c5ccccc5-6)cccc34)cccc21 ; CCC1(CC)c2ccccc2-c2ccc(N(c3cccc4c3C(C)(C)c3ccccc3-4)c3ccc4c(oc5ccc6ccc7ccccc7c6c54)c3-c3cccc(N(c4ccccc4)c4ccc(-c5ccc(-c6ccccc6)c(-c6ccccc6)c5)cc4)c3)cc21]

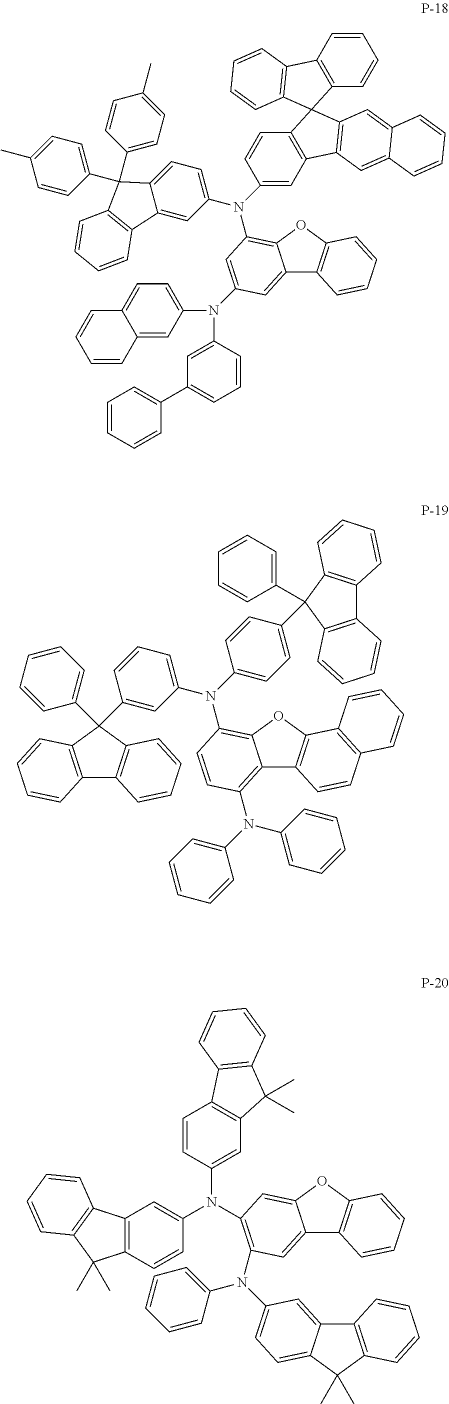 [SMILES: Cc1ccc(C2(c3ccc(C)cc3)c3ccccc3-c3cc(N(c4ccc5c(c4)-c4cc6ccccc6cc4C54c5ccccc5-c5ccccc54)c4cc(N(c5cccc(-c6ccccc6)c5)c5ccc6ccccc6c5)cc5c4oc4ccccc45)ccc32)cc1 ; c1ccc(N(c2ccccc2)c2ccc(N(c3ccc(C4(c5ccccc5)c5ccccc5-c5ccccc54)cc3)c3cccc(C4(c5ccccc5)c5ccccc5-c5ccccc54)c3)c3oc4c5ccccc5ccc4c23)cc1 ; CC1(C)c2ccccc2-c2cc(N(c3ccc4c(c3)C(C)(C)c3ccccc3-4)c3cc4oc5ccccc5c4cc3N(c3ccccc3)c3ccc4c(c3)-c3ccccc3C4(C)C)ccc21]

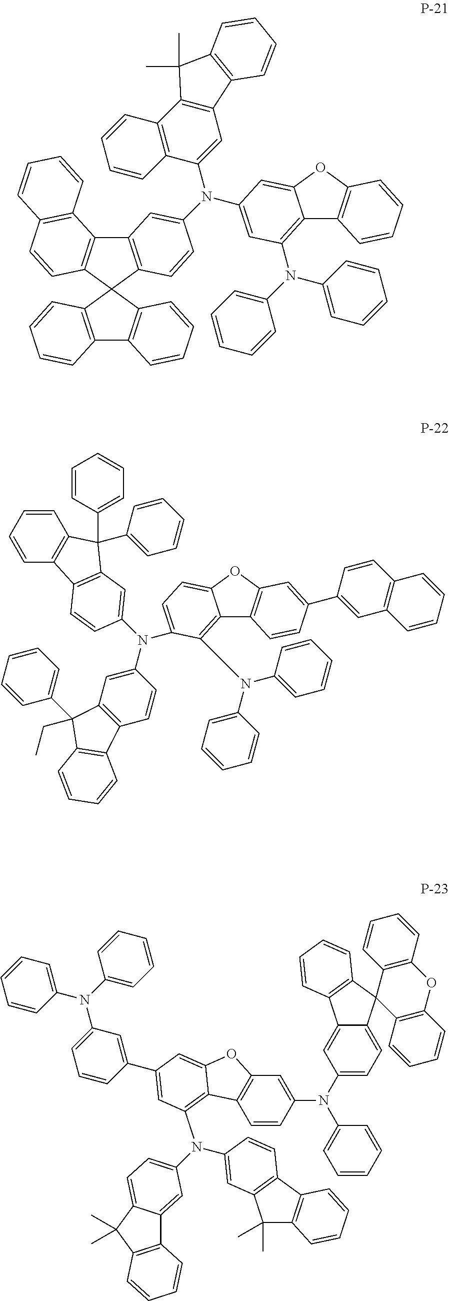 [SMILES: CC1(C)c2ccccc2-c2cc(N(c3ccc4c(c3)-c3c(ccc5ccccc35)C43c4ccccc4-c4ccccc43)c3cc(N(c4ccccc4)c4ccccc4)c4c(c3)oc3ccccc34)c3ccccc3c21 ; CCC1(c2ccccc2)c2ccccc2-c2ccc(N(c3ccc4c(c3)C(c3ccccc3)(c3ccccc3)c3ccccc3-4)c3ccc4oc5cc(-c6ccc7ccccc7c6)ccc5c4c3N(c3ccccc3)c3ccccc3)cc21 ; CC1(C)c2ccccc2-c2cc(N(c3ccc4c(c3)C(C)(C)c3ccccc3-4)c3cc(-c4cccc(N(c5ccccc5)c5ccccc5)c4)cc4oc5cc(N(c6ccccc6)c6ccc7c(c6)-c6ccccc6C76c7ccccc7Oc7ccccc76)ccc5c34)ccc21]

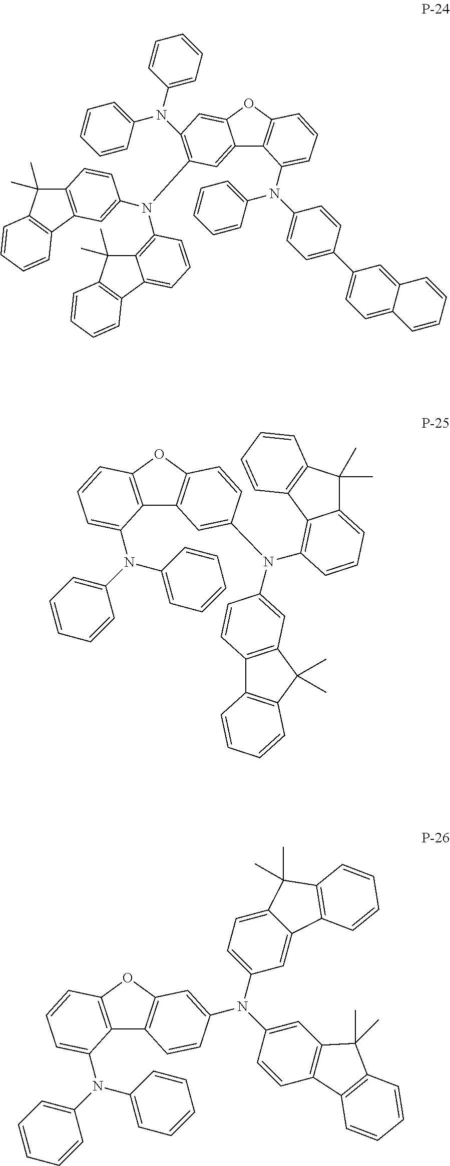 [SMILES: CC1(C)c2ccccc2-c2cc(N(c3cc4c(cc3N(c3ccccc3)c3ccccc3)oc3cccc(N(c5ccccc5)c5ccc(-c6ccc7ccccc7c6)cc5)c34)c3cccc4c3C(C)(C)c3ccccc3-4)ccc21 ; CC1(C)c2ccccc2-c2ccc(N(c3ccc4oc5cccc(N(c6ccccc6)c6ccccc6)c5c4c3)c3cccc4c3-c3ccccc3C4(C)C)cc21 ; CC1(C)c2ccccc2-c2cc(N(c3ccc4c(c3)C(C)(C)c3ccccc3-4)c3ccc4c(c3)oc3cccc(N(c5ccccc5)c5ccccc5)c34)ccc21]

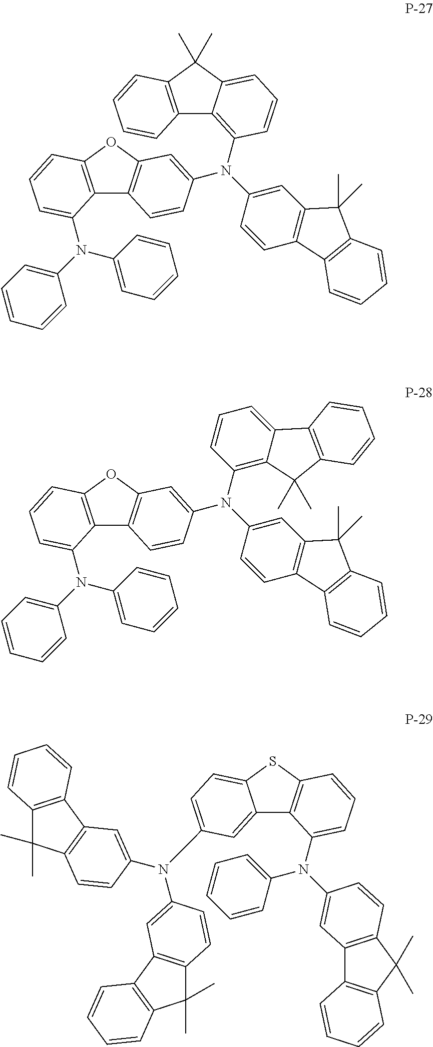

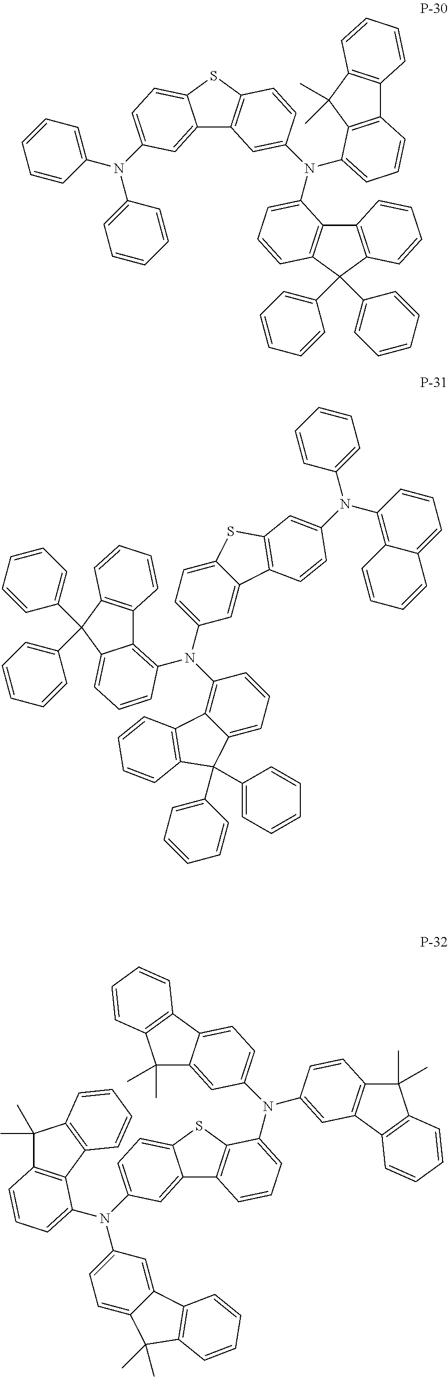 [SMILES: CC1(C)c2ccccc2-c2cccc(N(c3ccc4sc5ccc(N(c6ccccc6)c6ccccc6)cc5c4c3)c3cccc4c3-c3ccccc3C4(c3ccccc3)c3ccccc3)c21 ; c1ccc(N(c2ccc3c(c2)sc2ccc(N(c4cccc5c4-c4ccccc4C5(c4ccccc4)c4ccccc4)c4cccc5c4-c4ccccc4C5(c4ccccc4)c4ccccc4)cc23)c2cccc3ccccc23)cc1 ; CC1(C)c2ccccc2-c2cc(N(c3ccc4sc5c(N(c6ccc7c(c6)-c6ccccc6C7(C)C)c6ccc7c(c6)C(C)(C)c6ccccc6-7)cccc5c4c3)c3cccc4c3-c3ccccc3C4(C)C)ccc21]

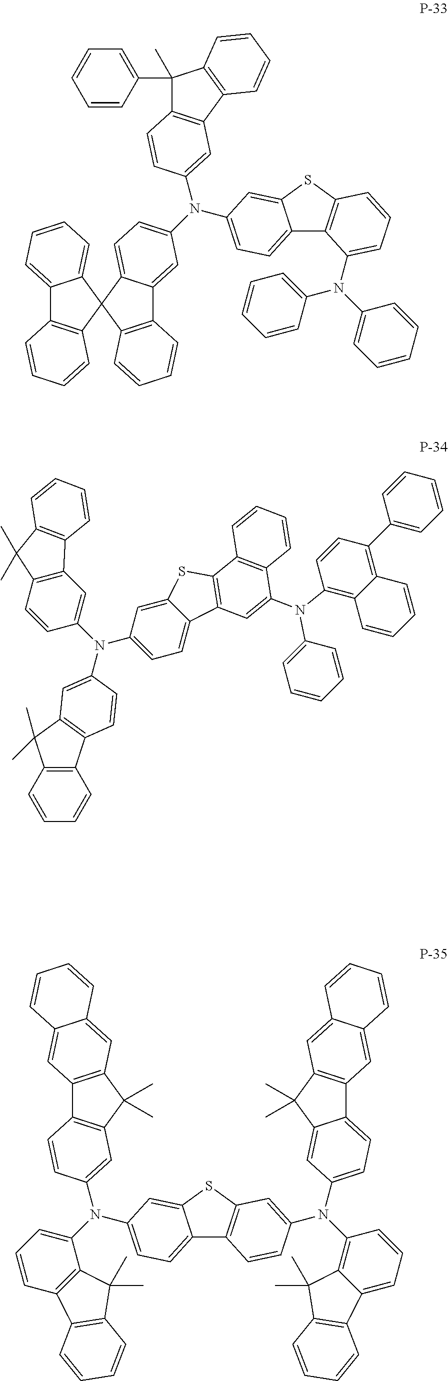 [SMILES: CC1(c2ccccc2)c2ccccc2-c2cc(N(c3ccc4c(c3)-c3ccccc3C43c4ccccc4-c4ccccc43)c3ccc4c(c3)sc3cccc(N(c5ccccc5)c5ccccc5)c34)ccc21 ; CC1(C)c2ccccc2-c2cc(N(c3ccc4c(c3)C(C)(C)c3ccccc3-4)c3ccc4c(c3)sc3c5ccccc5c(N(c5ccccc5)c5ccc(-c6ccccc6)c6ccccc56)cc43)ccc21 ; CC1(C)c2cc(N(c3ccc4c(c3)sc3cc(N(c5ccc6c(c5)C(C)(C)c5cc7ccccc7cc5-6)c5cccc6c5C(C)(C)c5ccccc5-6)ccc34)c3cccc4c3C(C)(C)c3ccccc3-4)ccc2-c2cc3ccccc3cc21]

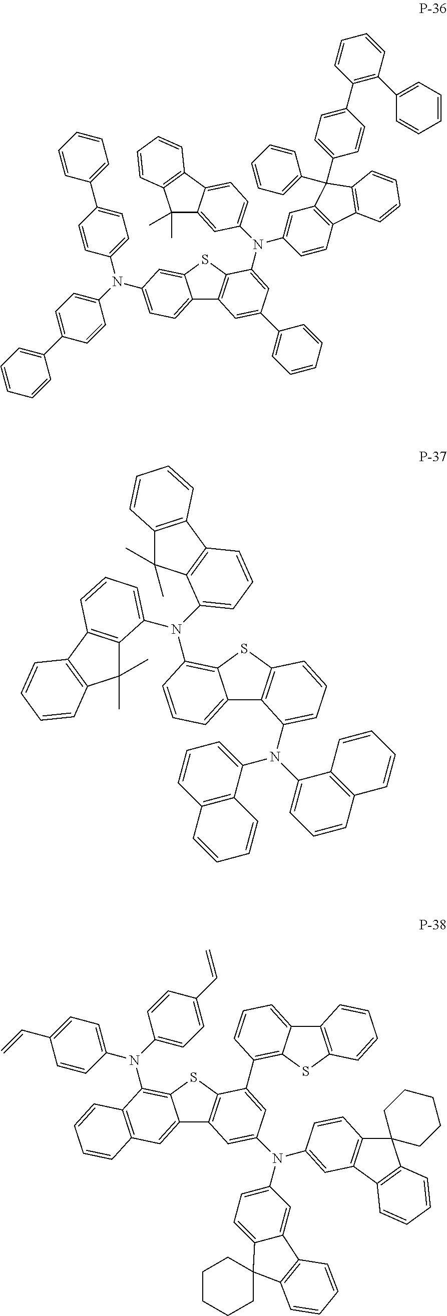 [SMILES: CC1(C)c2ccccc2-c2ccc(N(c3ccc4c(c3)C(c3ccccc3)(c3ccc(-c5ccccc5-c5ccccc5)cc3)c3ccccc3-4)c3cc(-c4ccccc4)cc4c3sc3cc(N(c5ccc(-c6ccccc6)cc5)c5ccc(-c6ccccc6)cc5)ccc34)cc21 ; CC1(C)c2ccccc2-c2cccc(N(c3cccc4c3C(C)(C)c3ccccc3-4)c3cccc4c3sc3cccc(N(c5cccc6ccccc56)c5cccc6ccccc56)c34)c21 ; C=Cc1ccc(N(c2ccc(C=C)cc2)c2c3ccccc3cc3c2sc2c(-c4cccc5c4sc4ccccc45)cc(N(c4ccc5c(c4)-c4ccccc4C54CCCCC4)c4ccc5c(c4)-c4ccccc4C54CCCCC4)cc23)cc1]

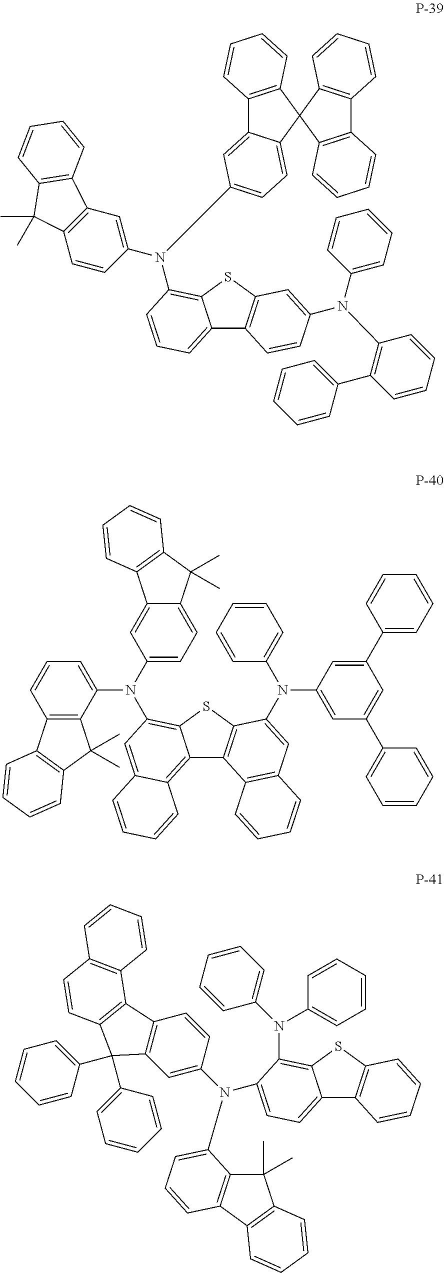 [SMILES: CC1(C)c2ccccc2-c2cc(N(c3ccc4c(c3)-c3ccccc3C43c4ccccc4-c4ccccc43)c3cccc4c3sc3cc(N(c5ccccc5)c5ccccc5-c5ccccc5)ccc34)ccc21 ; CC1(C)c2ccccc2-c2cc(N(c3cccc4c3C(C)(C)c3ccccc3-4)c3cc4ccccc4c4c3sc3c(N(c5ccccc5)c5cc(-c6ccccc6)cc(-c6ccccc6)c5)cc5ccccc5c34)ccc21 ; CC1(C)c2ccccc2-c2cccc(N(c3ccc4c(c3)C(c3ccccc3)(c3ccccc3)c3ccc5ccccc5c3-4)c3ccc4c(sc5ccccc54)c3N(c3ccccc3)c3ccccc3)c21]

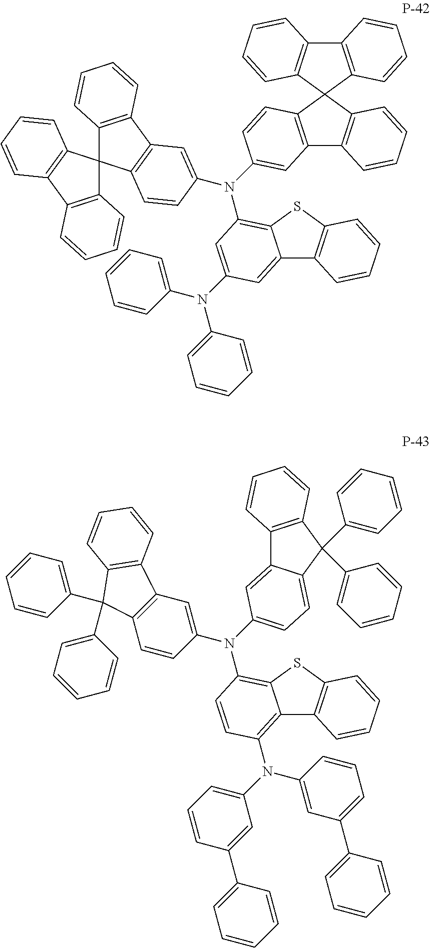 [SMILES: c1ccc(N(c2ccccc2)c2cc(N(c3ccc4c(c3)-c3ccccc3C43c4ccccc4-c4ccccc43)c3ccc4c(c3)-c3ccccc3C43c4ccccc4-c4ccccc43)c3sc4ccccc4c3c2)cc1 ; c1ccc(-c2cccc(N(c3cccc(-c4ccccc4)c3)c3ccc(N(c4ccc5c(c4)-c4ccccc4C5(c4ccccc4)c4ccccc4)c4ccc5c(c4)-c4ccccc4C5(c4ccccc4)c4ccccc4)c4sc5ccccc5c34)c2)cc1]

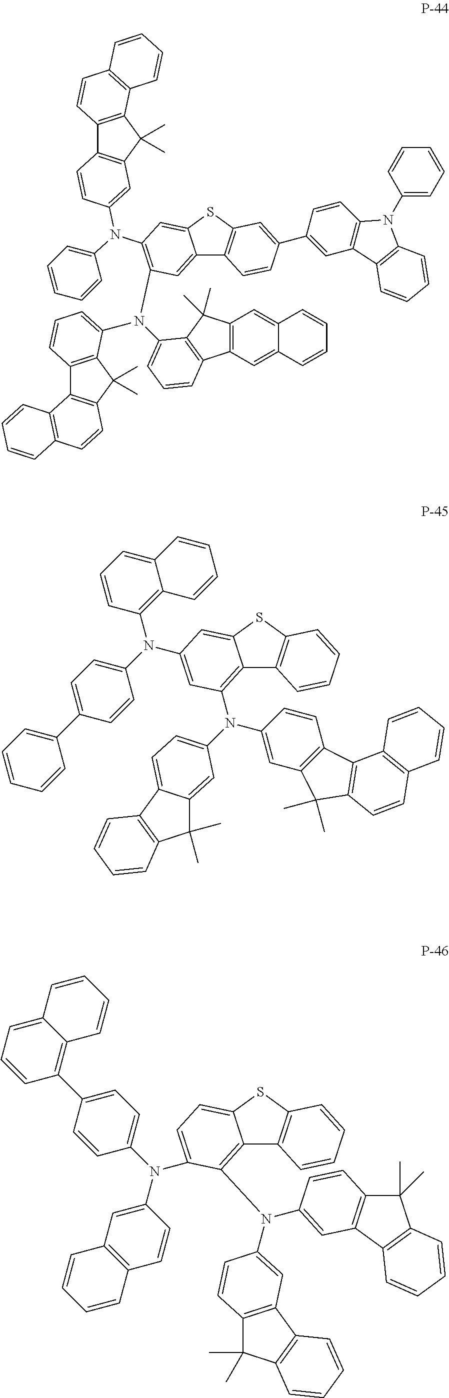 [SMILES: CC1(C)c2cc3ccccc3cc2-c2cccc(N(c3cc4c(cc3N(c3ccccc3)c3ccc5c(c3)C(C)(C)c3c-5ccc5ccccc35)sc3cc(-c5ccc6c(c5)c5ccccc5n6-c5ccccc5)ccc34)c3cccc4c3C(C)(C)c3ccc5ccccc5c3-4)c21 ; CC1(C)c2ccccc2-c2ccc(N(c3ccc4c(c3)C(C)(C)c3ccc5ccccc5c3-4)c3cc(N(c4ccc(-c5ccccc5)cc4)c4cccc5ccccc45)cc4sc5ccccc5c34)cc21 ; CC1(C)c2ccccc2-c2cc(N(c3ccc4c(c3)-c3ccccc3C4(C)C)c3c(N(c4ccc(-c5cccc6ccccc56)cc4)c4ccc5ccccc5c4)ccc4sc5ccccc5c34)ccc21]

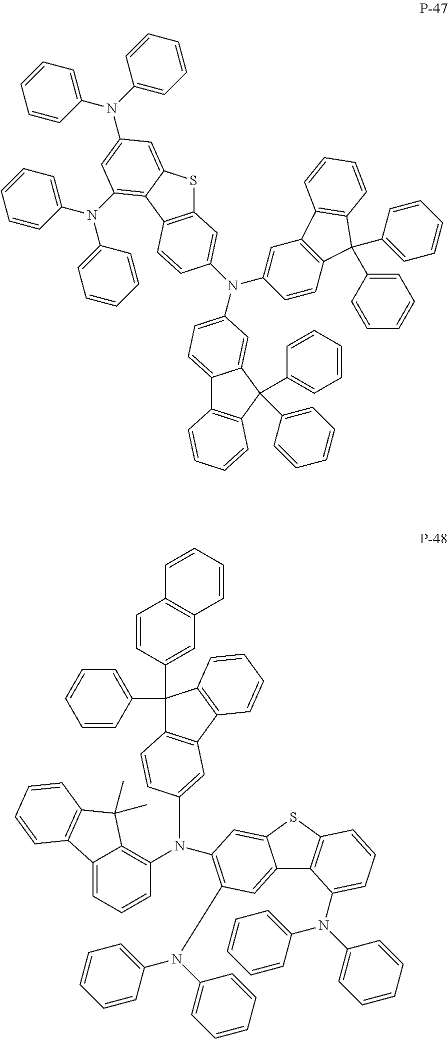 [SMILES: c1ccc(N(c2ccccc2)c2cc(N(c3ccccc3)c3ccccc3)c3c(c2)sc2cc(N(c4ccc5c(c4)-c4ccccc4C5(c4ccccc4)c4ccccc4)c4ccc5c(c4)C(c4ccccc4)(c4ccccc4)c4ccccc4-5)ccc23)cc1 ; CC1(C)c2ccccc2-c2cccc(N(c3ccc4c(c3)-c3ccccc3C4(c3ccccc3)c3ccc4ccccc4c3)c3cc4sc5cccc(N(c6ccccc6)c6ccccc6)c5c4cc3N(c3ccccc3)c3ccccc3)c21]

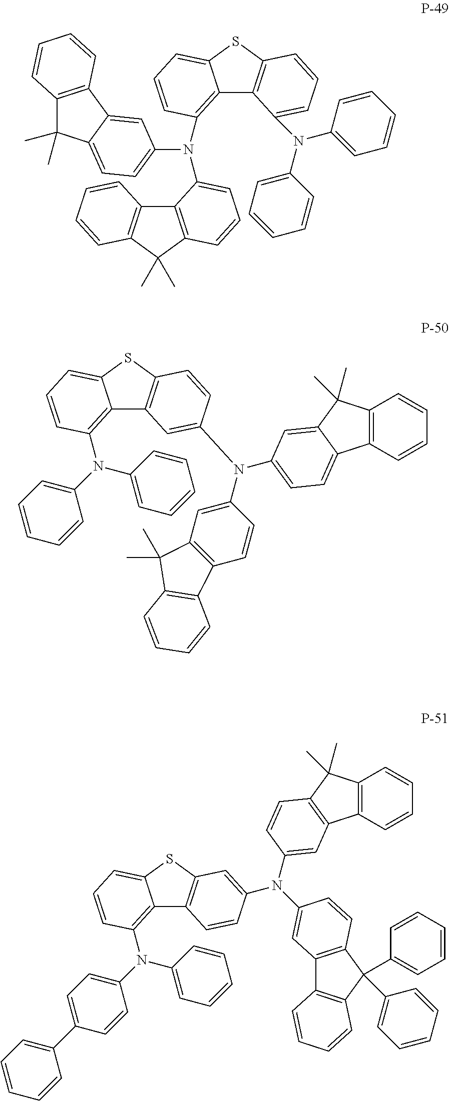 [SMILES: CC1(C)c2ccccc2-c2cc(N(c3cccc4c3-c3ccccc3C4(C)C)c3cccc4sc5cccc(N(c6ccccc6)c6ccccc6)c5c34)ccc21 ; CC1(C)c2ccccc2-c2ccc(N(c3ccc4c(c3)C(C)(C)c3ccccc3-4)c3ccc4sc5cccc(N(c6ccccc6)c6ccccc6)c5c4c3)cc21 ; CC1(C)c2ccccc2-c2cc(N(c3ccc4c(c3)-c3ccccc3C4(c3ccccc3)c3ccccc3)c3ccc4c(c3)sc3cccc(N(c5ccccc5)c5ccc(-c6ccccc6)cc5)c34)ccc21]

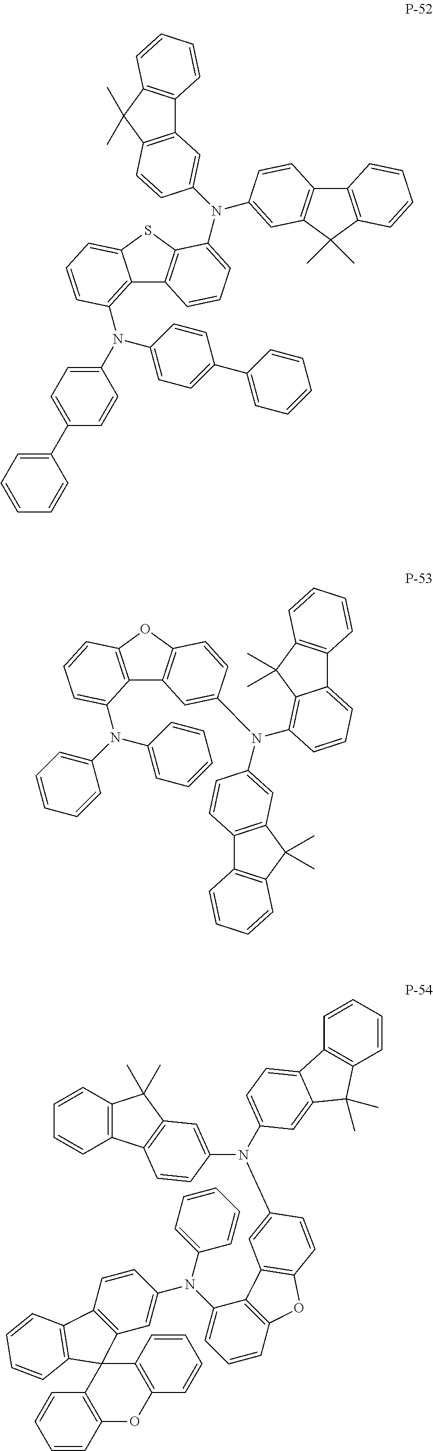 [SMILES: CC1(C)c2ccccc2-c2cc(N(c3ccc4c(c3)C(C)(C)c3ccccc3-4)c3cccc4c3sc3cccc(N(c5ccc(-c6ccccc6)cc5)c5ccc(-c6ccccc6)cc5)c34)ccc21 ; CC1(C)c2ccccc2-c2ccc(N(c3ccc4oc5cccc(N(c6ccccc6)c6ccccc6)c5c4c3)c3cccc4c3C(C)(C)c3ccccc3-4)cc21 ; CC1(C)c2ccccc2-c2ccc(N(c3ccc4c(c3)C(C)(C)c3ccccc3-4)c3ccc4oc5cccc(N(c6ccccc6)c6ccc7c(c6)C6(c8ccccc8Oc8ccccc86)c6ccccc6-7)c5c4c3)cc21]

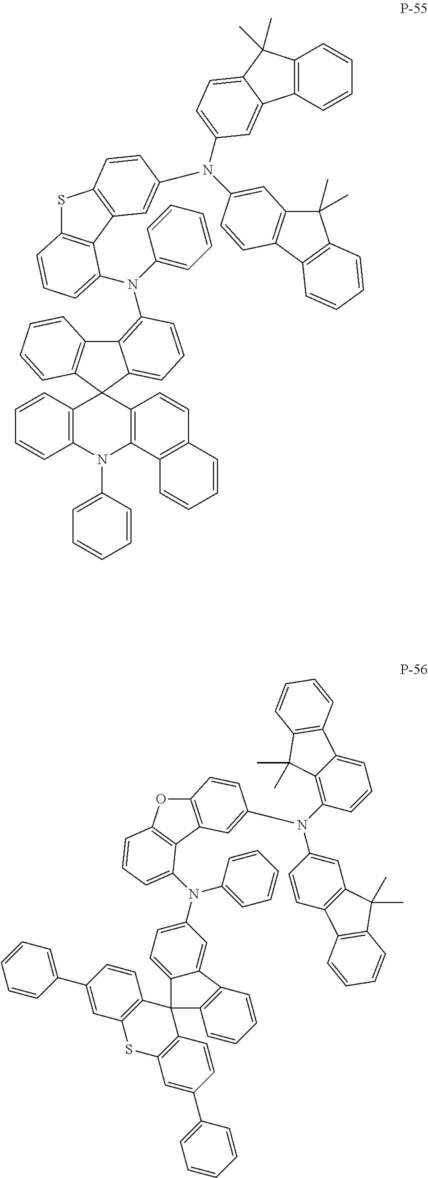 [SMILES: CC1(C)c2ccccc2-c2cc(N(c3ccc4c(c3)C(C)(C)c3ccccc3-4)c3ccc4sc5cccc(N(c6ccccc6)c6cccc7c6-c6ccccc6C76c7ccccc7N(c7ccccc7)c7c6ccc6ccccc76)c5c4c3)ccc21 ; CC1(C)c2ccccc2-c2ccc(N(c3ccc4oc5cccc(N(c6ccccc6)c6ccc7c(c6)-c6ccccc6C76c7ccc(-c8ccccc8)cc7Sc7cc(-c8ccccc8)ccc76)c5c4c3)c3cccc4c3C(C)(C)c3ccccc3-4)cc21]

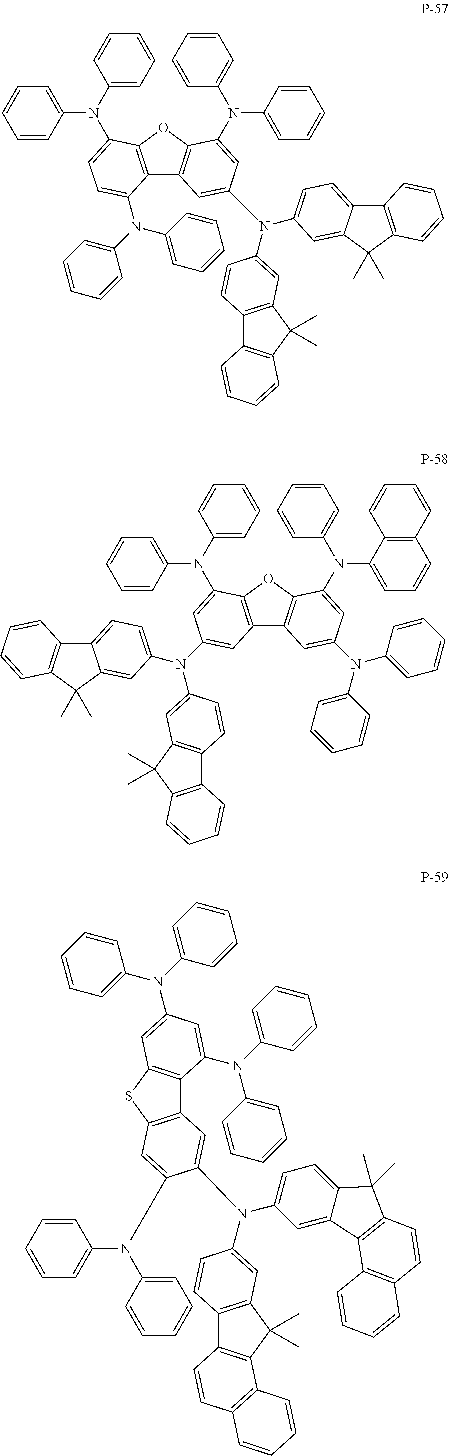 [SMILES: CC1(C)c2ccccc2-c2ccc(N(c3ccc4c(c3)C(C)(C)c3ccccc3-4)c3cc(N(c4ccccc4)c4ccccc4)c4oc5c(N(c6ccccc6)c6ccccc6)ccc(N(c6ccccc6)c6ccccc6)c5c4c3)cc21 ; CC1(C)c2ccccc2-c2ccc(N(c3ccc4c(c3)C(C)(C)c3ccccc3-4)c3cc(N(c4ccccc4)c4ccccc4)c4oc5c(N(c6ccccc6)c6cccc7ccccc67)cc(N(c6ccccc6)c6ccccc6)cc5c4c3)cc21 ; CC1(C)c2ccc(N(c3ccc4c(c3)C(C)(C)c3c-4ccc4ccccc34)c3cc4c(cc3N(c3ccccc3)c3ccccc3)sc3cc(N(c5ccccc5)c5ccccc5)cc(N(c5ccccc5)c5ccccc5)c34)cc2-c2c1ccc1ccccc21]

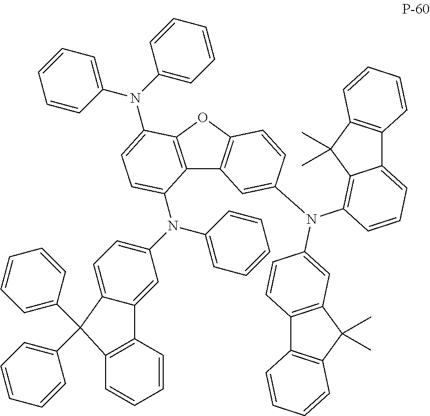 [SMILES: CC1(C)c2ccccc2-c2ccc(N(c3ccc4oc5c(N(c6ccccc6)c6ccccc6)ccc(N(c6ccccc6)c6ccc7c(c6)-c6ccccc6C7(c6ccccc6)c6ccccc6)c5c4c3)c3cccc4c3C(C)(C)c3ccccc3-4)cc21]

Referring to FIG. 1, the organic electronic element (100) according to the present invention includes a first electrode (110), a second electrode (170), and an organic material layer including a single compound or 2 or more compounds represented by Formula 1 between the first electrode (110) and the second electrode (170). In this case, the first electrode (110) may be an anode, and the second electrode (170) may be a cathode. In the case of an inverted type, the first electrode may be a cathode and the second electrode may be an anode.

The organic material layer may sequentially include a hole injection layer (120), a hole transport layer (130), an emitting layer (140), an electron transport layer (150), and an electron injection layer (160) on the first electrode (110). In this case, the remaining layers except for the emitting layer (140) may not be formed. It may further include a hole blocking layer, an electron blocking layer, an emitting-auxiliary layer (220), a buffer layer (210), etc. and the electron transport layer (150) and the like may serve as a hole blocking layer. (See FIG. 2)

Also, the organic electronic element according to an embodiment of the present invention may further include a protective layer or a light efficiency enhancing layer (180). The light efficiency enhancing layer may be formed on one of both surfaces of the first electrode not in contact with the organic material layer or on one of both surfaces of the second electrode not in contact with the organic material layer. The compound according to an embodiment of the present invention applied to the organic material layer may be used as a host or dopant of the hole injection layer (120), the hole transport layer (130), the emitting-auxiliary layer (220), electron transport auxiliary layer, the electron transport layer (150), and an electron injection layer (160), the emitting layer (140) or as a material for the light efficiency enhancing layer. Preferably, for example, the compound according to Formula 1 of the present invention may be used as a host of the emitting layer and/or as a material for the emitting auxiliary layer.

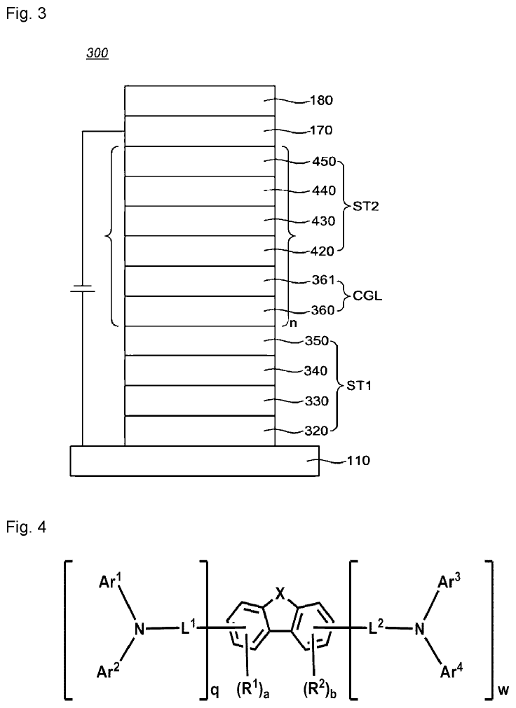

The organic material layer may include 2 or more stacks including a hole transport layer, an emitting layer and an electron transport layer sequentially formed on the anode, further include a charge generation layer formed between the 2 or more stacks (see FIG. 3).

Otherwise, even with the same core, the band gap, electrical characteristics, interface characteristics, etc. may vary depending on which position the substituent is bonded to, therefore the choice of core and the combination of sub-substituents bound thereto are also very important, and in particular, when the optimal combination of energy levels and T1 values and unique properties of materials(mobility, interfacial characteristics, etc.) of each organic material layer is achieved, a long lifespan and high efficiency can be achieved at the same time. The organic electroluminescent device according to an embodiment of the present invention may be manufactured using a PVD (physical vapor deposition) method. For example, depositing a metal or a metal oxide having conductivity or an alloy thereof on a substrate to form an anode, and after forming an organic material layer including the hole injection layer (120), the hole transport layer (130), the emitting layer (140), the electron transport layer (150) and the electron injection layer (160) thereon, it can be prepared by depositing a material that can be used as a cathode thereon.

Also, in the present invention, the organic material layer is formed by any one of a spin coating process, a nozzle printing process, an inkjet printing process, a slot coating process, a dip coating process, and a roll-to-roll process, and the organic material layer provides an organic electronic element comprising the compound as an electron transport material.

As another specific example, the same or different compounds of the compound represented by Formula 1 are mixed and used in the organic material layer.

Also, the present invention provides an emitting auxiliary layer composition comprising the compound represented by Formula 1, and provides an organic electronic element including the emitting auxiliary layer.

Also, the present invention provides a green organic electronic element comprising the compound represented by Formula 1.

Also, the present invention provides a green organic electronic element comprising an emitting auxiliary layer comprising the compound represented by Formula (1).

Also, the present invention provides an electronic device comprising a display device including the organic electronic element; and a control unit for driving the display device;

In another aspect, the organic electronic element is at least one of an organic electroluminescent device, an organic solar cell, an organic photo conductor, an organic transistor, and a device for monochromatic or white lighting. At this time, the electronic device may be a current or future wired/wireless communication terminal, and covers all kinds of electronic devices including mobile communication terminals such as mobile phones, a personal digital assistant (PDA), an electronic dictionary, a point-to-multipoint(PMP), a remote controller, a navigation unit, a game player, various kinds of TVs, and various kinds of computers.

Hereinafter, a synthesis example of the compound represented by Formula 1 of the present invention and a manufacturing example of an organic electronic element of the present invention will be described in detail with reference to Examples, but the present invention is not limited to the following Examples.

### SYNTHESIS EXAMPLE

The compound represented by Formula 1 according to the present invention (final products) is synthesized by reacting Sub 1 and Sub 2 as shown in Scheme 1, but is not limited thereto.

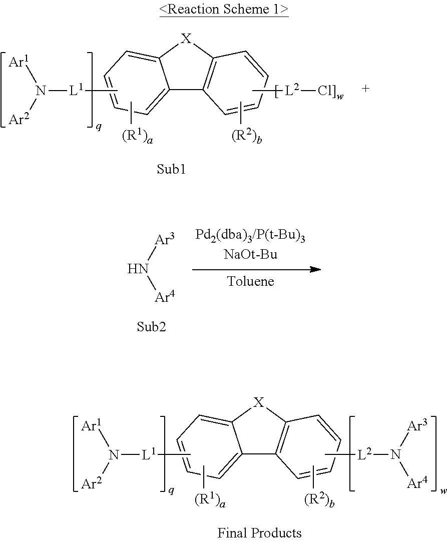

### I. Synthesis of Sub 1

Sub 1 of Reaction Scheme 1 may be synthesized by the reaction route of Scheme 2, but is not limited thereto.

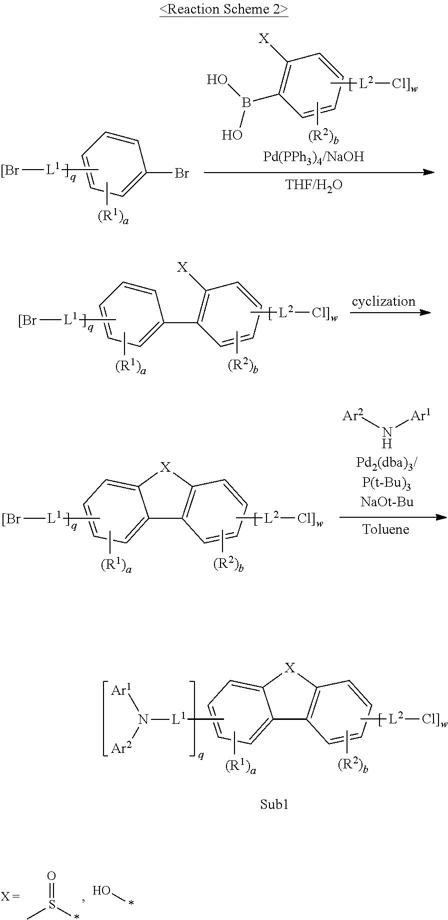

Synthesis examples of specific compounds belonging to Sub 1 are as follows.

### 1. Synthesis Example of Sub 1-1

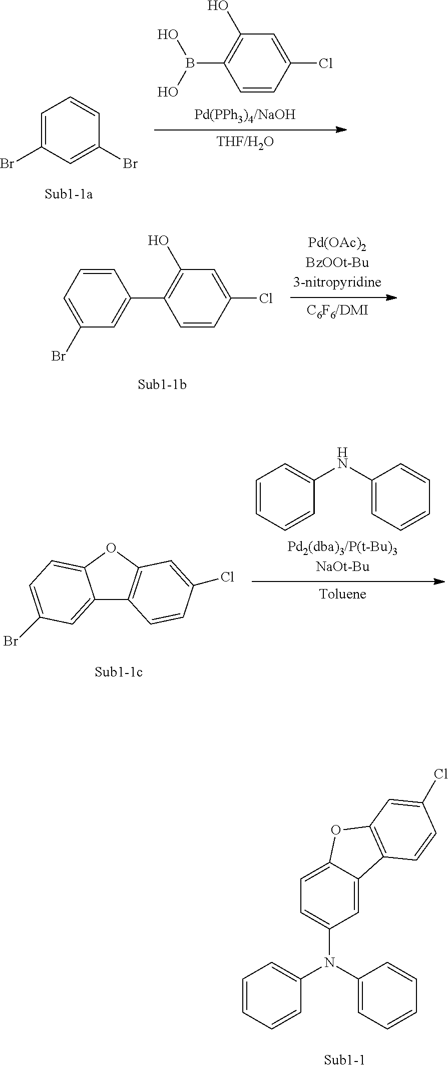 [SMILES: OB(O)c1ccc(Cl)cc1O ; Brc1cccc(Br)c1 ; Oc1cc(Cl)ccc1-c1cccc(Br)c1 ; c1ccc(Nc2ccccc2)cc1 ; Clc1ccc2c(c1)oc1ccc(Br)cc12 ; Clc1ccc2c(c1)oc1ccc(N(c3ccccc3)c3ccccc3)cc12]

(1) Synthesis of Sub 1-1 b

Sub 1-1a (50 g, 0.21 mol), (4-chloro-2-hydroxyphenyl)boronic acid (36.6 g, 0.21 mol), Pd(PPh₃)₄ (7.4 g, 0.006 mol), NaOH (25.5 g, 0.64 mol), THF (425 ml), Water (130 ml) were added and reacted for 6 hours. After the reaction was completed, the temperature of the reactant was cooled to room temperature and the reaction solvent was removed. Then, the concentrated reactant was separated using a silicagel column or recrystallization method to obtain 49 g (81.2%) of the product Sub 1-1b.

(2) Synthesis of Sub 1-1c

The obtained Sub 1-1 b (50 g, 0.18 mol) was added with Pd(OAc)₂ (1.2 g, 0.005 mol) and 3-nitropyridine (0.66 g, 0.005 mol), After dissolving in C₆F₆ (280 ml) and DMI (350 ml), tert-butyl peroxybenzoate (102.75 g, 0.53 mol) was added and stirred at 90° C. When the reaction was completed, 40 g (yield: 80.6%) of the product Sub 1-1c was obtained by using the separation method of Sub 1-1b.

(3) Synthesis of Sub 1-1

Diphenylamine (18 g, 0.11 mol), Pd₂(dba)₃ (2.9 g, 0.0032 mol), 50% P(t-Bu)₃ (2.6 g, 0.0064 mol), NaOt-Bu (30.8 g, 0.32 mol), Toluene (220 ml) were added to the obtained Sub 1-1c (30 g, 0.11 mol) and stirred at 90° C. When the reaction was completed, 55 g (yield: 90.6%) of the product Sub 1-1 was obtained by using the separation method of Sub 1-1b.

### 2. Synthesis Example of Sub 1-6

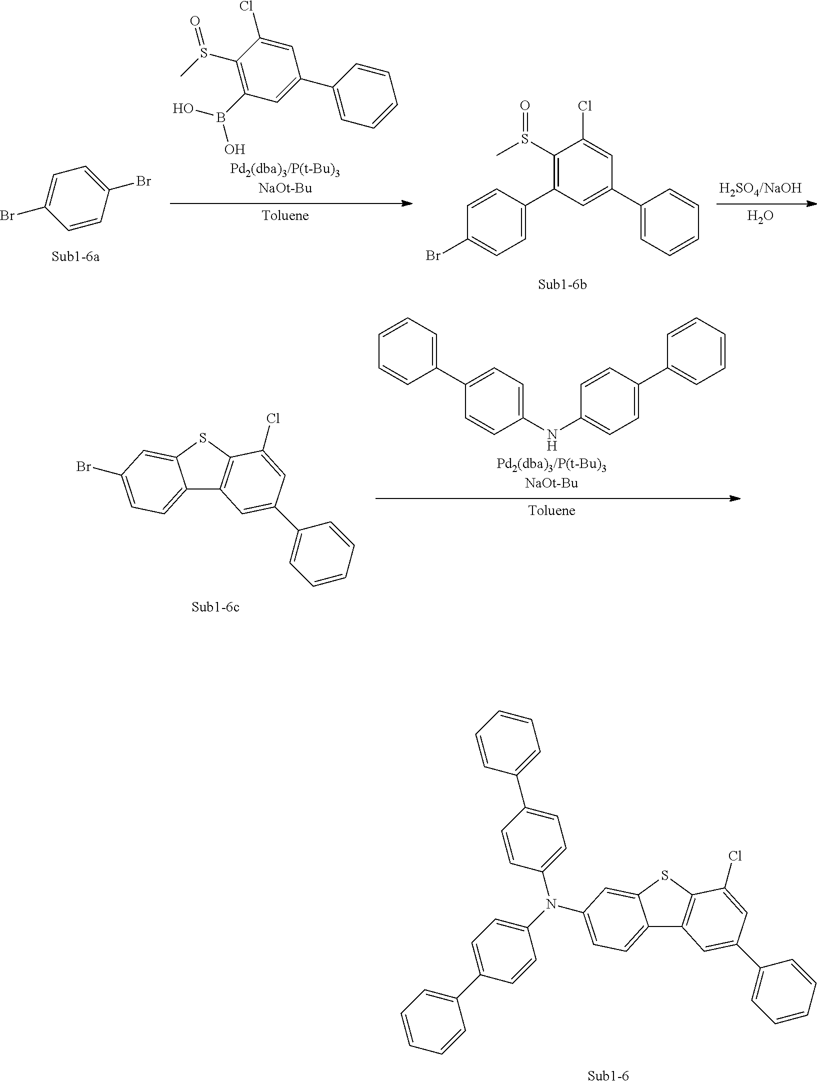

(1) Synthesis of Sub 1-6b

Sub 1-6a (60 g, 0.25 mol), (5-chloro-4-(methylsulfinyl)-[1,1′-biphenyl]-3-yl)boronic acid (75 g, 0.25 mol), Pd(PPh₃)₄ (7 g, 0.007 mol), NaOH (30.5 g, 0.76 mol), THF (510 ml) and Water (150 ml) were added and reacted for 6 hours.

After the reaction was completed, 88 g (85.4%) of Sub 1-6b was obtained by using the separation method of Sub 1-1b.

### (2) Synthesis of Sub 1-6c

Sub 1-6b (30 g, 0.07 mol) was dissolved in H₂SO₄ (150 ml) and stirred at 45° C. for 12 hours. When the reaction was completed, the reactant was slowly added to an aqueous NaOH solution and then neutralized. Then, 23 g (yield: 83.2%) of the product Sub 1-6c was obtained by using a silicagel column or recrystallization method for the produced reactant.

### (3) Synthesis of Sub 1-6

Di([1,1′-biphenyl]-4-yl)amine (43 g, 0.13 mol), Pd₂(dba)₃ (3.7 g, 0.004 mol), 50% P(t-Bu)₃ (3.3 g, 0.008 mol), NaOt-Bu (38.7 g, 0.4 mol), toluene (270 ml) were added to the obtained Sub 1-6c (50 g, 0.13 mol) and stirred at 90° C.

When the reaction was completed, 40 g (yield: 80.6%) of the product Sub 1-6 was obtained by using the separation method of Sub 1-1b.

### 3. Synthesis Example of Sub 1-14

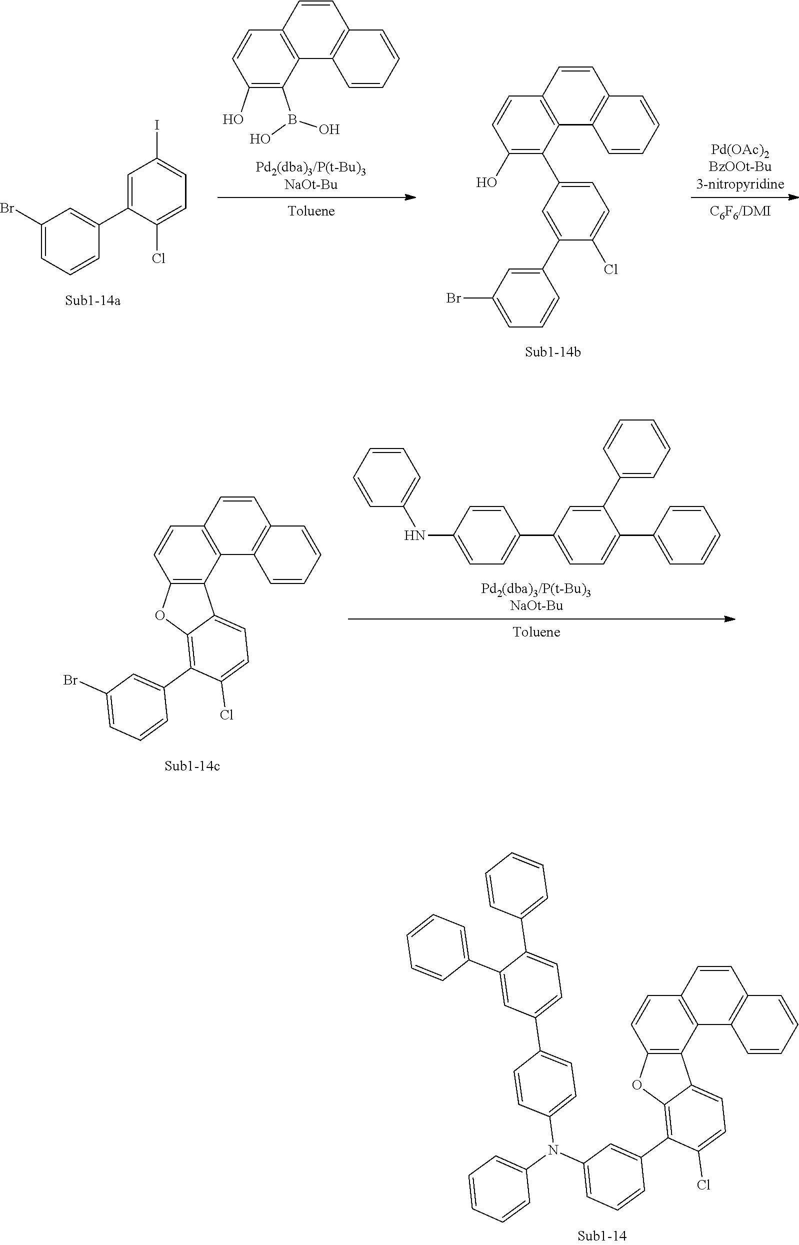 [SMILES: Clc1ccc(I)cc1-c1cccc(Br)c1 ; OB(O)c1c(O)ccc2ccc3ccccc3c12 ; Oc1ccc2ccc3ccccc3c2c1-c1ccc(Cl)c(-c2cccc(Br)c2)c1 ; Clc1ccc2c(oc3ccc4ccc5ccccc5c4c32)c1-c1cccc(Br)c1 ; c1ccc(Nc2ccc(-c3ccc(-c4ccccc4)c(-c4ccccc4)c3)cc2)cc1 ; Clc1ccc2c(oc3ccc4ccc5ccccc5c4c32)c1-c1cccc(N(c2ccccc2)c2ccc(-c3ccc(-c4ccccc4)c(-c4ccccc4)c3)cc2)c1]

### (1) Synthesis of Sub 1-14b

Sub 1-14a (30 g, 0.08 mol), (3-hydroxyphenanthren-4-yl)boronic acid (18 g, 0.08 mol), Pd(PPh₃)₄ (2.6 g, 0.002 mol), NaOH (9.2 g, 0.23 mol), THF (150 ml) and Water (40 ml) were added and reacted for 6 hours. After the reaction was completed, the temperature of the reactant was cooled to room temperature and the reaction solvent was removed. After the reaction was completed, 31 g (yield: 88.5%) of the product Sub 1-14b was obtained by using the separation method of Sub 1-1b.

### (2) Synthesis of Sub 1-14c

The obtained Sub 1-14b (30 g, 0.07 mol) was put together with Pd(OAc)₂ (0.44 g, 0.002 mol), 3-nitropyridine (0.24 g, 0.002 mol), dissolved in C₆F₆ (90 ml), DMI (130 ml), tent-butyl peroxybenzoate (38.1 g, 0.196 mol) was added, and the mixture was stirred at 90° C. When the reaction was completed, 27 g (yield: 90.4%) of the product Sub 1-14c was obtained by using the separation method of Sub 1-1b.

### (3) Synthesis of Sub 1-14

n, 4′-diphenyl-[1,1′: 3′, 1″-terphenyl]-4-amine (13 g, 0.05 mol), Pd₂(dba)₃ (1.5 g, 0.0016 mol), 50% P(t-Bu)3 (1.3 g, 0.0033 mol), NaOt-Bu (15.8 g, 0.16 mol), toluene (110 ml) were added to the obtained Sub 1-14c (25 g, 0.05 mol)

and stirred at 90° C. When the reaction was completed, 35 g (yield: 82.7%) of the product Sub 1-14 was obtained by using the separation method of Sub 1-1b.

4. Synthesis example of Sub 1-16

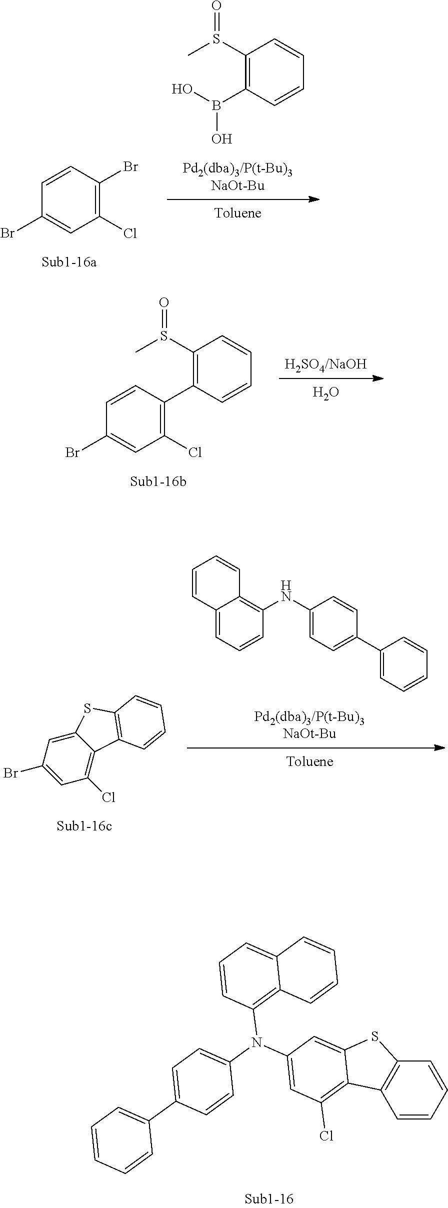

(1) Synthesis of Sub 1-16b

Sub 1-16a (30 g, 0.11 mol), (2-(methylsulfinyl)phenyl)boronic acid (20 g, 0.11 mol), Pd(PPh₃)₄ (3.9 g, 0.003 mol), NaOH (13.3 g, 0.33 mol), THF (220 ml) and Water (70 ml) were added and reacted for 6 hours. After the reaction was completed, 30 g (82.2%) of the product Sub 1-16b was obtained by using the separation method of Sub 1-1b.

(2) Synthesis of Sub 1-16c

The obtained Sub 1-16b (30 g, 0.09 mol) was dissolved in H₂SO₄ (150 ml), followed by stirring at 45° C. for 12 hours. When the reaction was completed, the reactant was slowly added to an aqueous NaOH solution and then neutralized. Then, 24 g (yield: 88.4%) of the product Sub 1-16c was obtained by using a silicagel column or recrystallization method for the resulting reactant.

### (3) Synthesis of Sub 1-16

N-([1,1′-biphenyl]-4-yl)naphthalen-1-amine (29.8 g, 0.10 mol), Pd₂(dba)₃ (2.8 g, 0.003 mol), 50% P(t-Bu)3 (2.4 g, 0.006 mol), NaOt-Bu (29.1 g, 0.3 mol), Toluene (200 ml) were added to the obtained Sub 1-16c (30 g, 0.10 mol) and stirred at 90° C. When the reaction was completed, 44 g (yield: 85.2%) of the product Sub 1-16 was obtained by using the separation method of Sub 1-1b.

### 5. Synthesis Example of Sub 1-26

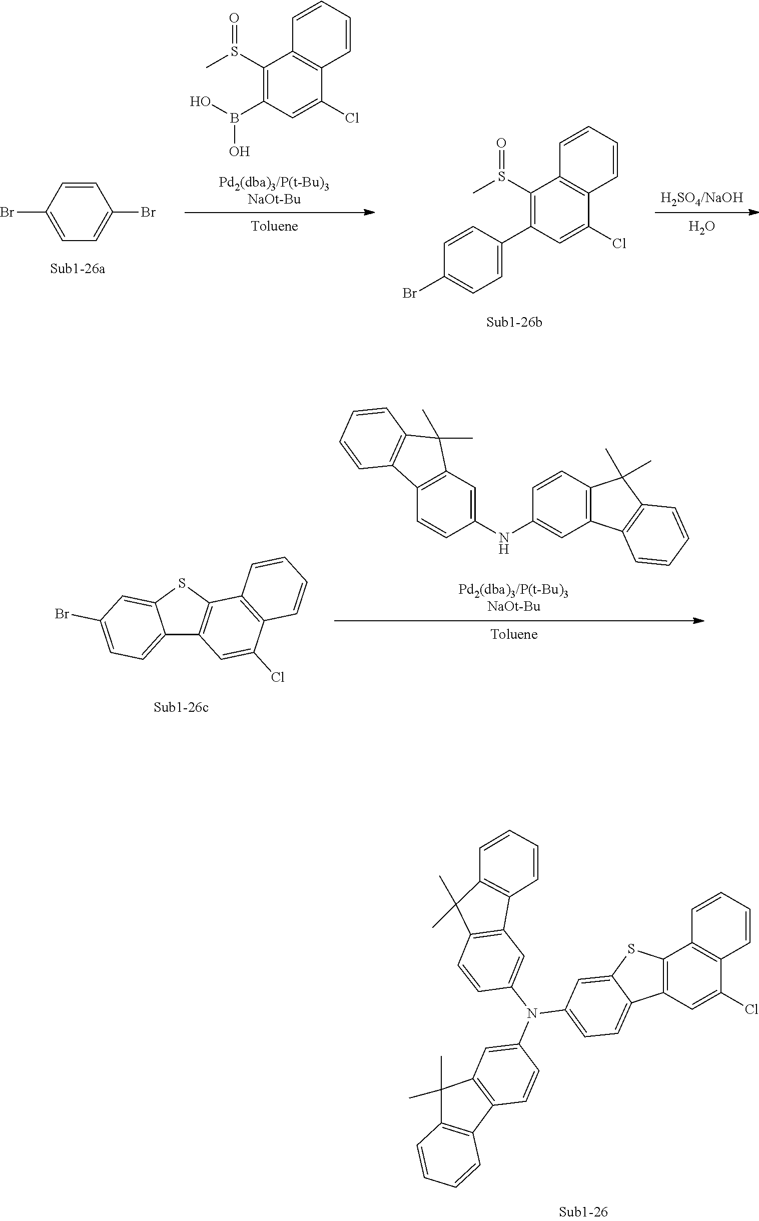

### (1) Synthesis of Sub 1-26b

Sub 1-26a (40 g, 0.17 mol), (4-chloro-1-(methylsulfinyl)naphthalen-2-yl)boronic acid (45.5 g, 0.17 mol), Pd(PPh₃)₄ (5.9 g, 0.005 mol), NaOH (20.3 g, 0.51 mol), THF (340 ml), Water (120 ml) were added and reacted for 6 hours.

After the reaction was completed, 51 g (79.4%) of Sub 1-26b was obtained by using the separation method of Sub 1-1b.

### (2) Synthesis of Sub 1-26c

The obtained Sub 1-26b (38 g, 0.10 mol) was dissolved in H₂SO₄ (200 ml) and stirred at 45° C. for 12 hours. When the reaction was completed, the reactant was slowly added to an aqueous NaOH solution and then neutralized. Then, 31 g (yield: 88.9%) of the product Sub 1-26c was obtained by using a silicagel column or recrystallization method for the resulting reactant.

### (3) Synthesis of Sub 1-26

N-(9,9-dimethyl-9H-fluoren-3-yl)-9,9-dimethyl-9H-fluoren-2-amine (35.8 g, 0.09 mol), Pd₂(dba)₃ (2.5 g, 0.003 mol), 50% P(t-Bu)₃ (2.2 g, 0.006 mol), NaOt-Bu (25.7 g, 0.3 mol), Toluene (180 ml) were added to the obtained Sub 1-26c (31 g, 0.09 mol) and stirred at 90° C. When the reaction was completed, 48 g (yield: 80.5%) of the product Sub 1-26 was obtained by using the separation method of Sub 1-1b.

### 6. Synthesis Example of Sub 1-35

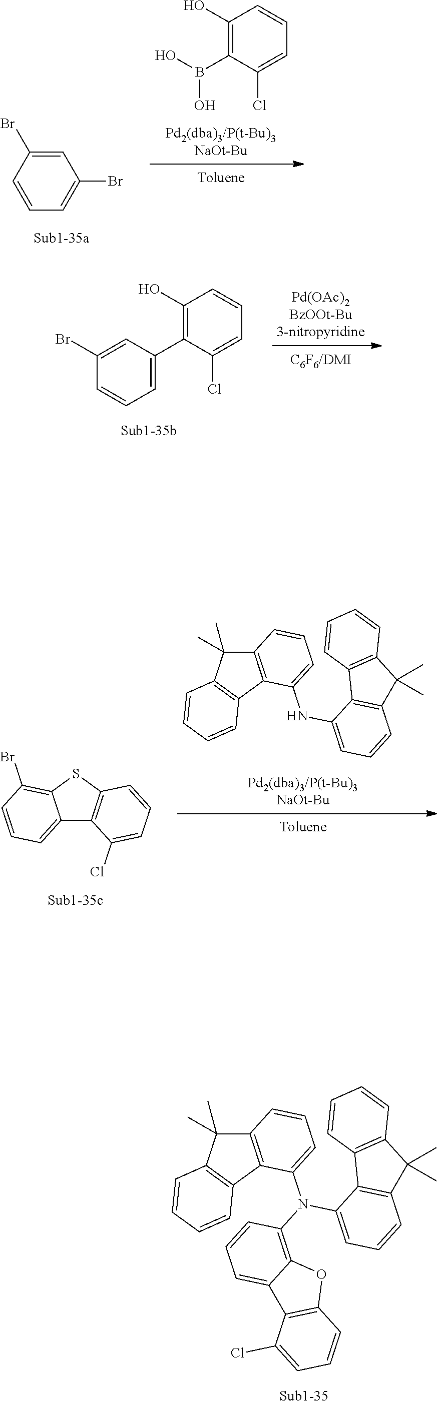

### (1) Synthesis of Sub 1-35b

Sub 1-35a (40 g, 0.17 mol), (2-chloro-6-hydroxyphenyl)boronic acid (29.2 g, 0.17 mol), Pd(PPh₃)₄ (5.9 g, 0.005 mol), NaOH (20.3 g, 0.51 mol), THF (340 ml), Water (110 ml) were added and reacted for 6 hours. After the reaction was completed, the temperature of the reactant was cooled to room temperature and the reaction solvent was removed. After the reaction was completed, 41 g (85.3%) of Sub 1-35b was obtained by using the separation method of Sub 1-1b.

### (2) Synthesis of Sub 1-35c

The obtained Sub 1-35b (40 g, 0.14 mol) was put together with Pd(OAc)₂ (0.95 g, 0.004 mol), 3-nitropyridine (0.53 g, 0.004 mol), dissolved in C₆F₆ (170 ml) and DMI (280 ml), tert-butyl peroxybenzoate (82.2 g, 0.423 mol) was added, and the mixture was stirred at 90° C. When the reaction was completed, 34 g (yield: 85.6%) of the product Sub 1-35c was obtained by using the separation method of Sub 1-1b.

### (3) Synthesis of Sub 1-35

bis(9,9-dimethyl-9H-fluoren-4-yl)amine (50 g, 0.12 mol), Pd₂(dba)₃ (3.4 g, 0.0037 mol), 50% P(t-Bu)₃ (3 g, 0.0075 mol), NaOt-Bu (35.9 g, 0.37 mol), toluene (250 ml) were added to the obtained Sub 1-35c (35 g, 0.12 mol) and stirred at 90° C. When the reaction was completed, 60 g (yield: 80%) of the product Sub 1-35 was obtained by using the separation method of Sub 1-1b.

7. Synthesis example of Sub 1-48

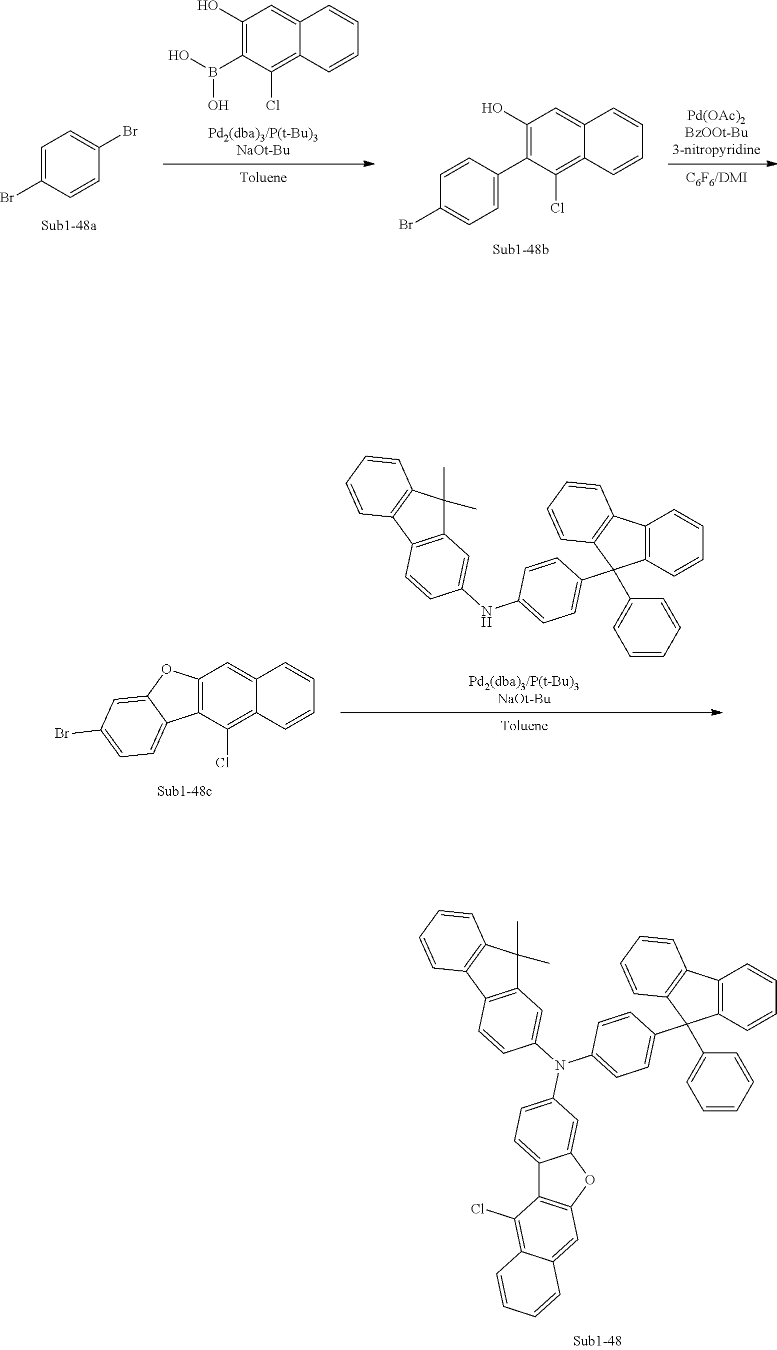

### (1) Synthesis of Sub 1-48b

Sub 1-48a (30 g, 0.13 mol), (1-chloro-3-hydroxynaphthalen-2-yl)boronic acid (28.3 g, 0.13 mol), Pd(PPh₃)₄ (4.4 g, 0.004 mol), NaOH (15.3 g, 0.38 mol), THF (250 ml), and Water (80 ml) were added and reacted for 6 hours. After the reaction was completed, the temperature of the reactant was cooled to room temperature and the reaction solvent was removed. After the reaction was completed, 35 g (yield: 82.5%) of the product Sub 1-48b was obtained by using the separation method of Sub 1-1b.

### (2) Synthesis of Sub 1-48c

The obtained Sub 1-48b (35 g, 0.10 mol) was put together with Pd(OAc)₂ (0.71 g, 0.003 mol), 3-nitropyridine (0.39 g, 0.003 mol), dissolved in C₆F₆ (160 ml), DMI (210 ml), tent-butyl peroxybenzoate (61.1 g, 0.32 mol) was added, and the mixture was stirred at 90° C. When the reaction was completed, 30 g (yield: 86.4%) of the product Sub 1-48c was obtained by using the separation method of Sub 1-1b.

### (3) Synthesis of Sub 1-48

9,9-dimethyl-N-(4-(9-phenyl-9H-fluoren-9-yl)phenyl)-9H-fluoren-2-amine (55.5 g, 0.11 mol), Pd2(dba)₃ (2.9 g, 0.0032 mol), 50% P(t-Bu)₃ (2.6 g, 0.0064 mol), NaOt-Bu (30.5 g, 0.32 mol), toluene (210 ml) were added to the obtained Sub 1-14c (25 g, 0.05 mol) and stirred at 90° C. When the reaction was completed, 70 g (yield: 85.3%) of the product Sub 1-48 was obtained by using the separation method of Sub 1-1b.

### 8. Synthesis Example of Sub 1-49

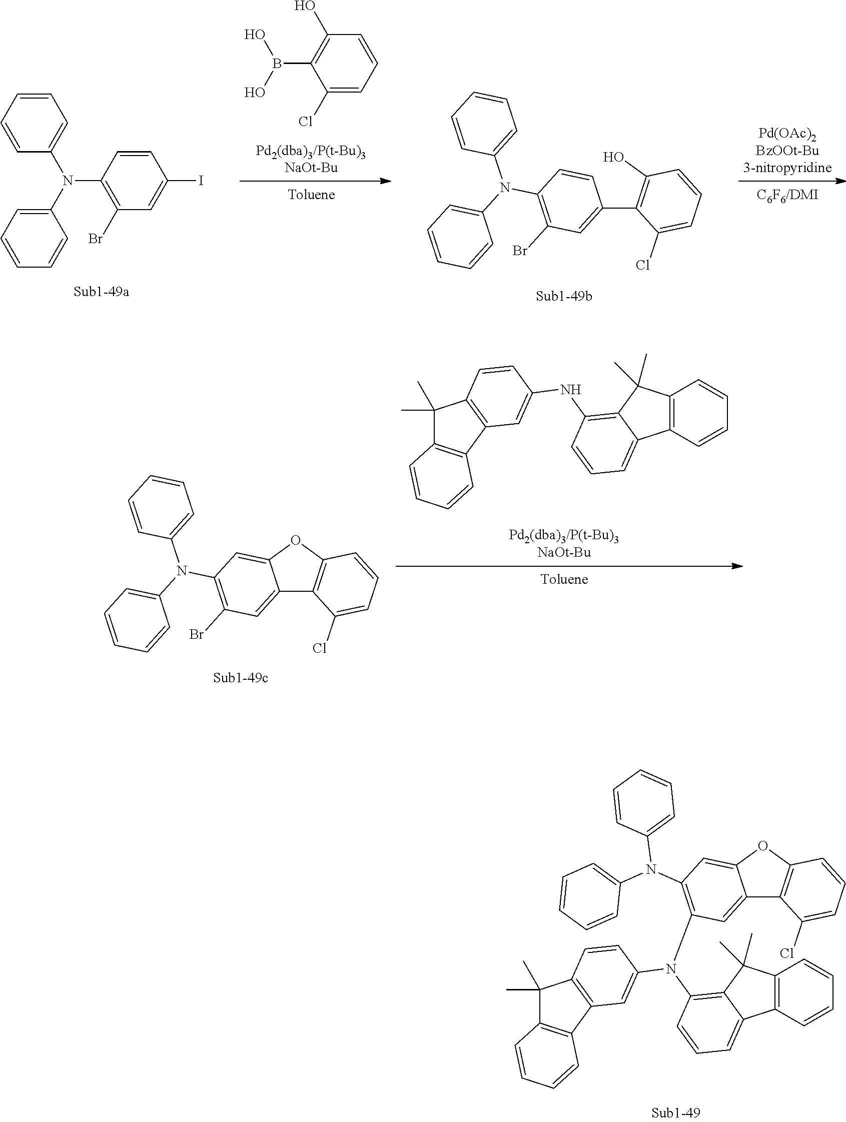 [SMILES: Brc1cc(I)ccc1N(c1ccccc1)c1ccccc1 ; OB(O)c1c(O)cccc1Cl ; Oc1cccc(Cl)c1-c1ccc(N(c2ccccc2)c2ccccc2)c(Br)c1 ; Clc1cccc2oc3cc(N(c4ccccc4)c4ccccc4)c(Br)cc3c12 ; CC1(C)c2ccccc2-c2cc(Nc3cccc4c3C(C)(C)c3ccccc3-4)ccc21 ; CC1(C)c2ccccc2-c2cc(N(c3cc4c(cc3N(c3ccccc3)c3ccccc3)oc3cccc(Cl)c34)c3cccc4c3C(C)(C)c3ccccc3-4)ccc21]

### (1) Synthesis of Sub 1-49b

Sub 1-49a (40 g, 0.09 mol), (2-chloro-6-hydroxyphenyl)boronic acid (15.3 g, 0.09 mol), Pd(PPh₃)₄ (3.1 g, 0.003 mol), NaOH (10.7 g, 0.27 mol), THF (180 ml) and water (60 ml) were added and reacted for 6 hours. After the reaction was completed, the temperature of the reactant was cooled to room temperature and the reaction solvent was removed. After the reaction was completed, 34 g (yield: 85%) of the product Sub 1-49b was obtained by using the separation method of Sub 1-1b.

### (2) Synthesis of Sub 1-49c

The obtained Sub 1-49b (30 g, 0.07 mol) was put together with Pd(OAc)₂ (0.45 g, 0.002 mol), 3-nitropyridine (0.25 g, 0.002 mol), dissolved in C₆F₆ (70 ml), DMI (130 ml), tent-butyl peroxybenzoate (38.8 g, 0.2 mol) was added, and the mixture was stirred at 90° C. When the reaction was completed, 27 g (yield: 90.3%) of the product Sub 1-49c was obtained by using the separation method of Sub 1-1b.

### (3) Synthesis of Sub 1-49

N-(9,9-dimethyl-9H-fluoren-3-yl)-9,9-dimethyl-9H-fluoren-1-amine (44.8 g, 0.11 mol), Pd₂(dba)₃ (3.1 g, 0.0033 mol), 50% P(t-Bu)3 (2.7 g, 0.007 mol), NaOt-Bu (32.2 g, 0.33 mol), toluene (230 ml) were added to the obtained Sub 1-49c (50 g, 0.11 mol) and stirred at 90° C. When the reaction was completed, 74 g (yield: 86.2%) of the product Sub 1-49 was obtained by using the separation method of Sub 1-1b.

The compound belonging to Sub 1 may be the following compounds, but is not limited thereto, and Table 1 below shows FD-MS values of the compounds belonging to Sub 1.

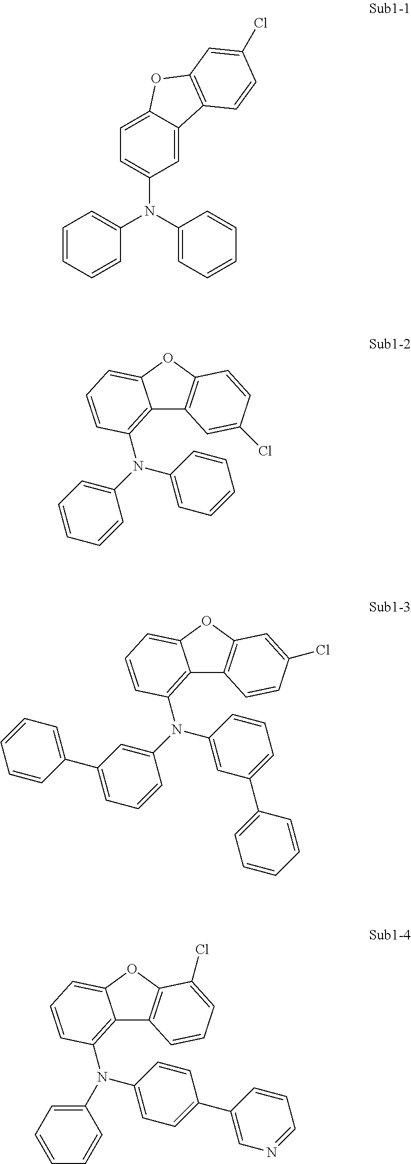 [SMILES: Clc1ccc2c(c1)oc1ccc(N(c3ccccc3)c3ccccc3)cc12 ; Clc1ccc2oc3cccc(N(c4ccccc4)c4ccccc4)c3c2c1 ; Clc1ccc2c(c1)oc1cccc(N(c3cccc(-c4ccccc4)c3)c3cccc(-c4ccccc4)c3)c12 ; Clc1cccc2c1oc1cccc(N(c3ccccc3)c3ccc(-c4cccnc4)cc3)c12]

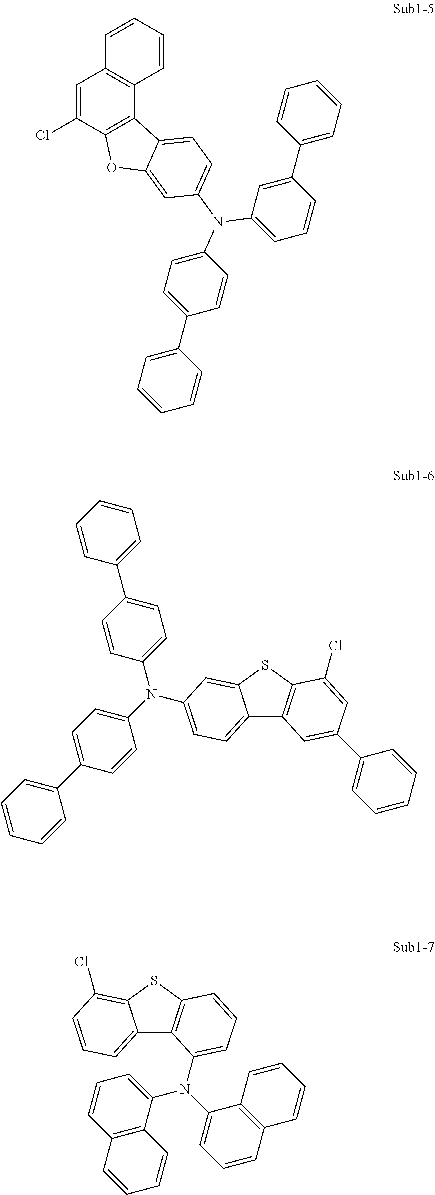 [SMILES: Clc1cc2ccccc2c2c1oc1cc(N(c3ccc(-c4ccccc4)cc3)c3cccc(-c4ccccc4)c3)ccc12 ; Clc1cc(-c2ccccc2)cc2c1sc1cc(N(c3ccc(-c4ccccc4)cc3)c3ccc(-c4ccccc4)cc3)ccc12 ; Clc1cccc2c1sc1cccc(N(c3cccc4ccccc34)c3cccc4ccccc34)c12]

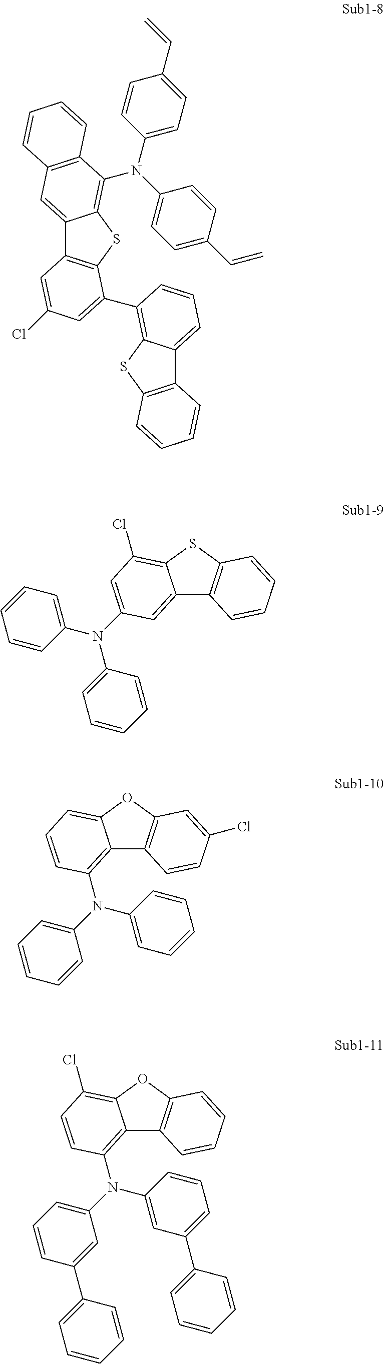 [SMILES: C=Cc1ccc(N(c2ccc(C=C)cc2)c2c3ccccc3cc3c2sc2c(-c4cccc5c4sc4ccccc45)cc(Cl)cc23)cc1 ; Clc1cc(N(c2ccccc2)c2ccccc2)cc2c1sc1ccccc12 ; Clc1ccc2c(c1)oc1cccc(N(c3ccccc3)c3ccccc3)c12 ; Clc1ccc(N(c2cccc(-c3ccccc3)c2)c2cccc(-c3ccccc3)c2)c2c1oc1ccccc12]

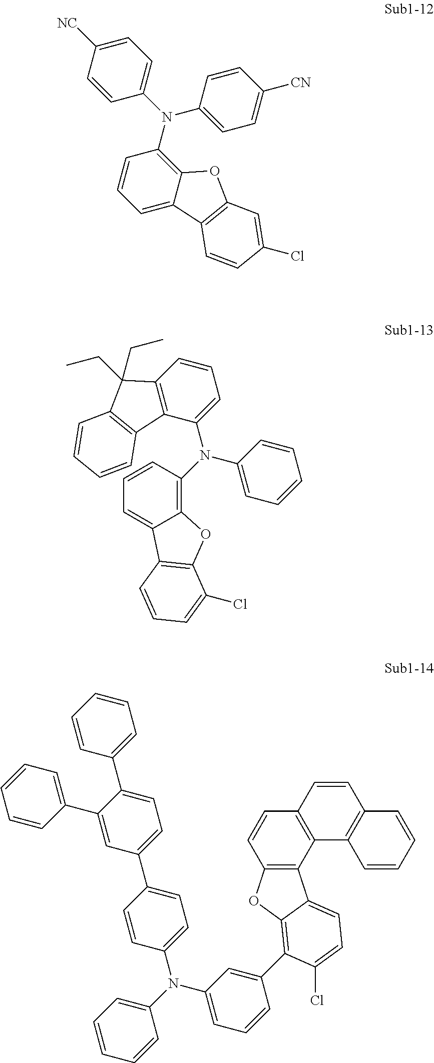 [SMILES: N#Cc1ccc(N(c2ccc(C#N)cc2)c2cccc3c2oc2cc(Cl)ccc23)cc1 ; CCC1(CC)c2ccccc2-c2c(N(c3ccccc3)c3cccc4c3oc3c(Cl)cccc34)cccc21 ; Clc1ccc2c(oc3ccc4ccc5ccccc5c4c32)c1-c1cccc(N(c2ccccc2)c2ccc(-c3ccc(-c4ccccc4)c(-c4ccccc4)c3)cc2)c1]

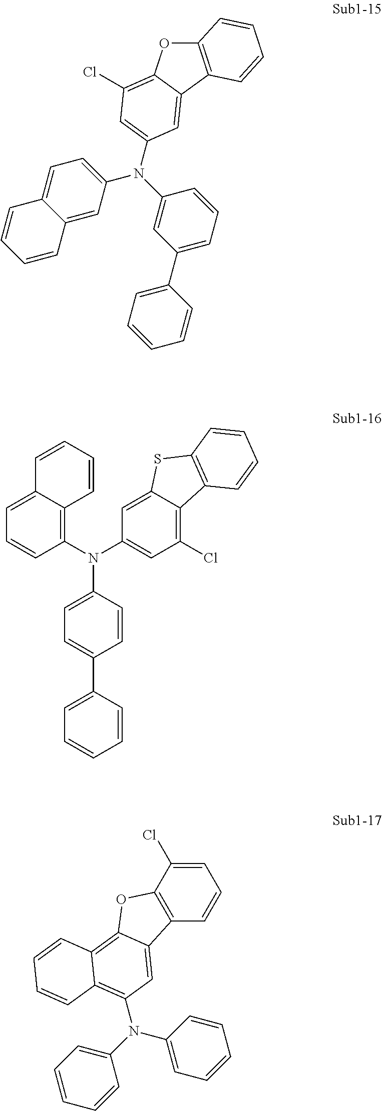 [SMILES: Clc1cc(N(c2cccc(-c3ccccc3)c2)c2ccc3ccccc3c2)cc2c1oc1ccccc12 ; Clc1cc(N(c2ccc(-c3ccccc3)cc2)c2cccc3ccccc23)cc2sc3ccccc3c12 ; Clc1cccc2c1oc1c3ccccc3c(N(c3ccccc3)c3ccccc3)cc21]

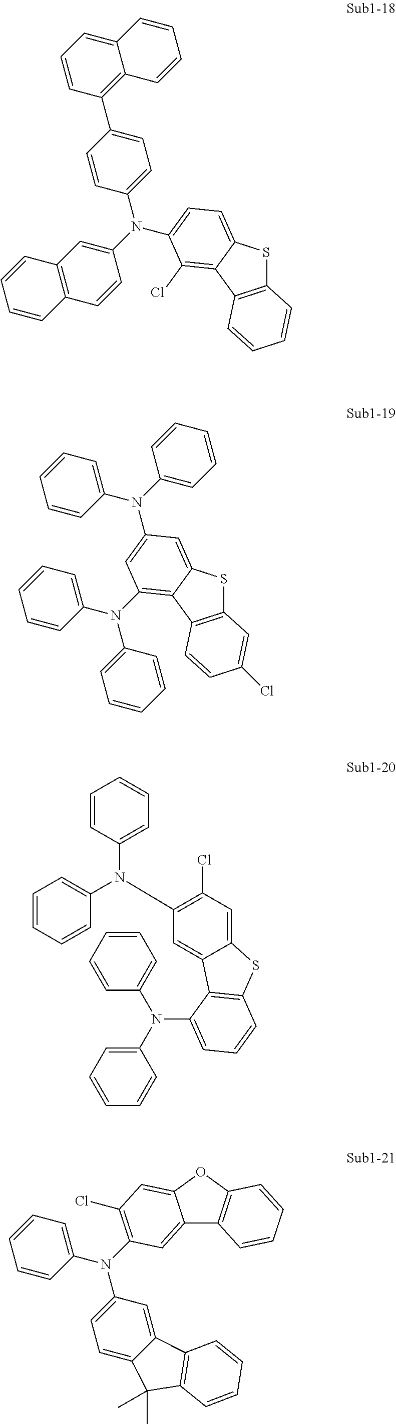 [SMILES: Clc1c(N(c2ccc(-c3cccc4ccccc34)cc2)c2ccc3ccccc3c2)ccc2sc3ccccc3c12 ; Clc1ccc2c(c1)sc1cc(N(c3ccccc3)c3ccccc3)cc(N(c3ccccc3)c3ccccc3)c12 ; Clc1cc2sc3cccc(N(c4ccccc4)c4ccccc4)c3c2cc1N(c1ccccc1)c1ccccc1 ; CC1(C)c2ccccc2-c2cc(N(c3ccccc3)c3cc4c(cc3Cl)oc3ccccc34)ccc21]

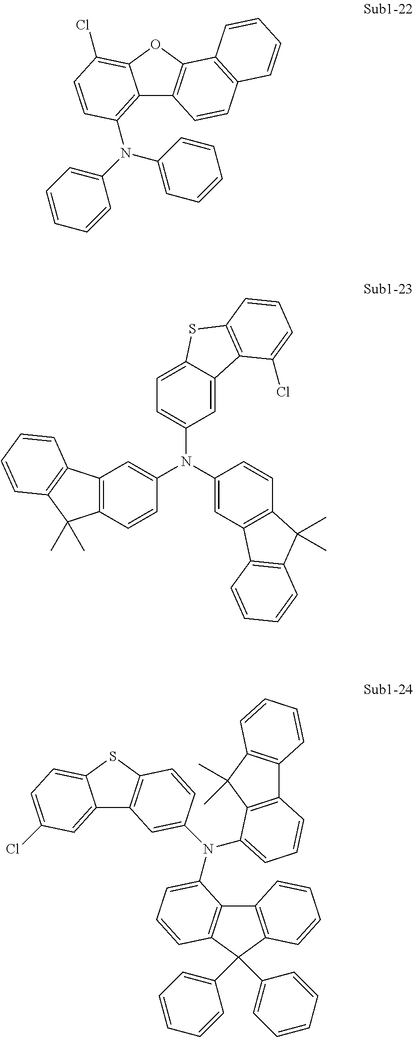 [SMILES: Clc1ccc(N(c2ccccc2)c2ccccc2)c2c1oc1c3ccccc3ccc12 ; CC1(C)c2ccccc2-c2cc(N(c3ccc4c(c3)-c3ccccc3C4(C)C)c3ccc4sc5cccc(Cl)c5c4c3)ccc21 ; CC1(C)c2ccccc2-c2cccc(N(c3ccc4sc5ccc(Cl)cc5c4c3)c3cccc4c3-c3ccccc3C4(c3ccccc3)c3ccccc3)c21]

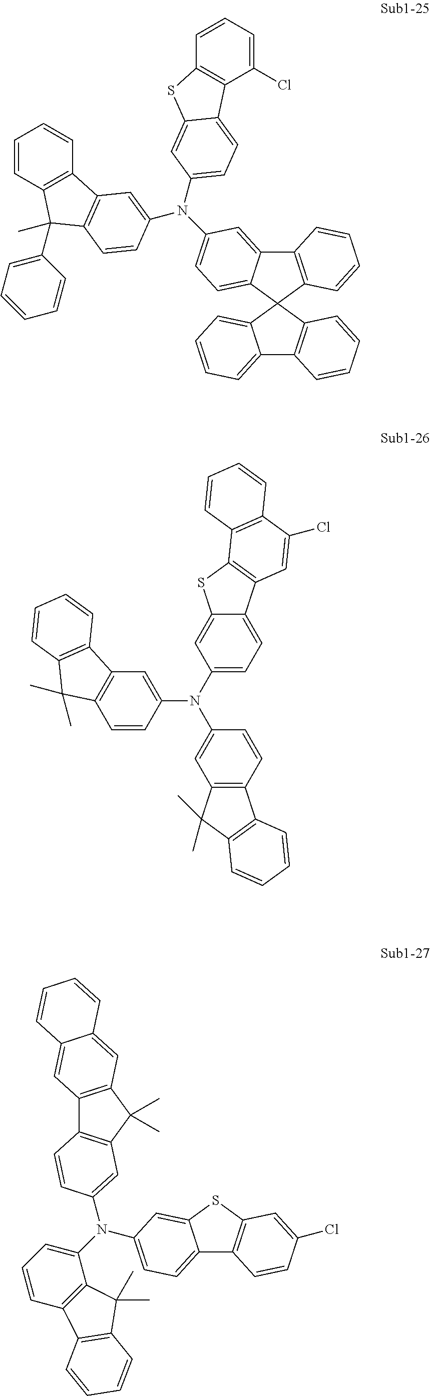 [SMILES: CC1(c2ccccc2)c2ccccc2-c2cc(N(c3ccc4c(c3)-c3ccccc3C43c4ccccc4-c4ccccc43)c3ccc4c(c3)sc3cccc(Cl)c34)ccc21 ; CC1(C)c2ccccc2-c2cc(N(c3ccc4c(c3)C(C)(C)c3ccccc3-4)c3ccc4c(c3)sc3c5ccccc5c(Cl)cc43)ccc21 ; CC1(C)c2cc(N(c3ccc4c(c3)sc3cc(Cl)ccc34)c3cccc4c3C(C)(C)c3ccccc3-4)ccc2-c2cc3ccccc3cc21]

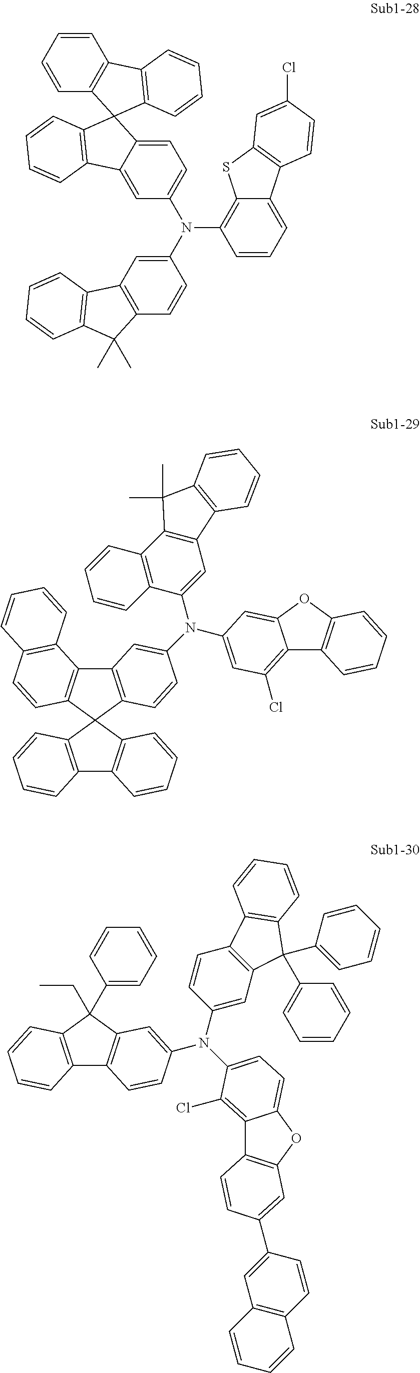 [SMILES: CC1(C)c2ccccc2-c2cc(N(c3ccc4c(c3)-c3ccccc3C43c4ccccc4-c4ccccc43)c3cccc4c3sc3cc(Cl)ccc34)ccc21 ; CC1(C)c2ccccc2-c2cc(N(c3ccc4c(c3)-c3c(ccc5ccccc35)C43c4ccccc4-c4ccccc43)c3cc(Cl)c4c(c3)oc3ccccc34)c3ccccc3c21 ; CCC1(c2ccccc2)c2ccccc2-c2ccc(N(c3ccc4c(c3)C(c3ccccc3)(c3ccccc3)c3ccccc3-4)c3ccc4oc5cc(-c6ccc7ccccc7c6)ccc5c4c3Cl)cc21]

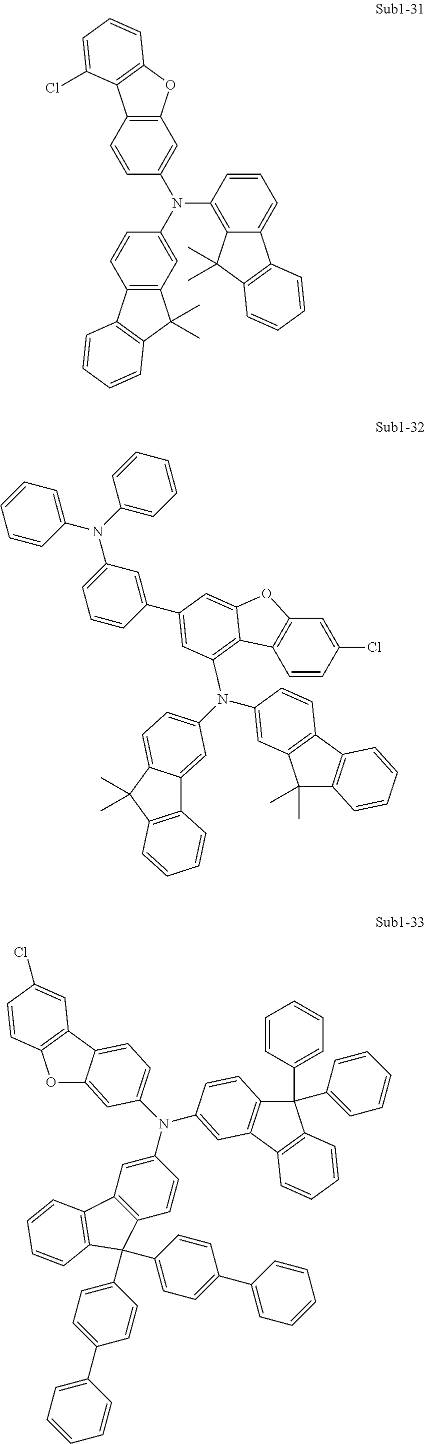 [SMILES: CC1(C)c2ccccc2-c2ccc(N(c3ccc4c(c3)oc3cccc(Cl)c34)c3cccc4c3C(C)(C)c3ccccc3-4)cc21 ; CC1(C)c2ccccc2-c2cc(N(c3ccc4c(c3)C(C)(C)c3ccccc3-4)c3cc(-c4cccc(N(c5ccccc5)c5ccccc5)c4)cc4oc5cc(Cl)ccc5c34)ccc21 ; Clc1ccc2oc3cc(N(c4ccc5c(c4)-c4ccccc4C5(c4ccccc4)c4ccccc4)c4ccc5c(c4)-c4ccccc4C5(c4ccc(-c5ccccc5)cc4)c4ccc(-c5ccccc5)cc4)ccc3c2c1]

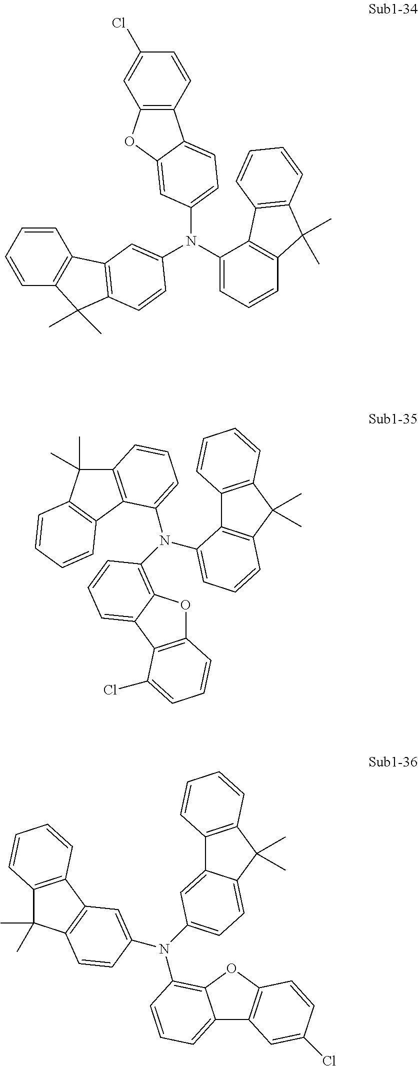

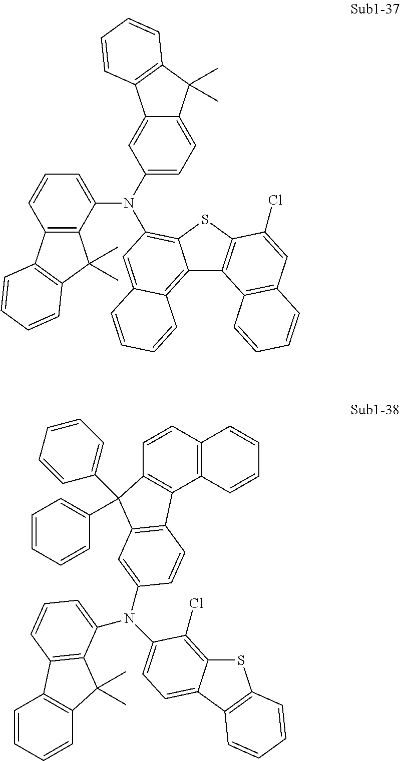 [SMILES: CC1(C)c2ccccc2-c2cc(N(c3cccc4c3C(C)(C)c3ccccc3-4)c3cc4ccccc4c4c3sc3c(Cl)cc5ccccc5c34)ccc21 ; CC1(C)c2ccccc2-c2cccc(N(c3ccc4c(c3)C(c3ccccc3)(c3ccccc3)c3ccc5ccccc5c3-4)c3ccc4c(sc5ccccc54)c3Cl)c21]

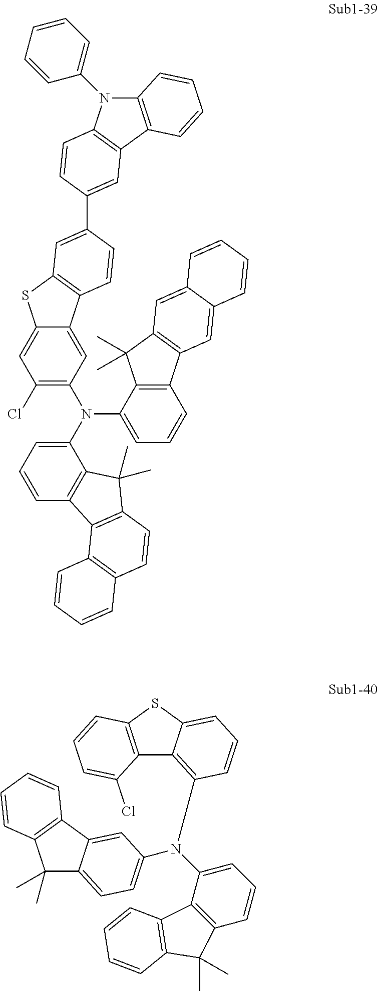 [SMILES: CC1(C)c2cc3ccccc3cc2-c2cccc(N(c3cc4c(cc3Cl)sc3cc(-c5ccc6c(c5)c5ccccc5n6-c5ccccc5)ccc34)c3cccc4c3C(C)(C)c3ccc5ccccc5c3-4)c21 ; CC1(C)c2ccccc2-c2cc(N(c3cccc4c3-c3ccccc3C4(C)C)c3cccc4sc5cccc(Cl)c5c34)ccc21]

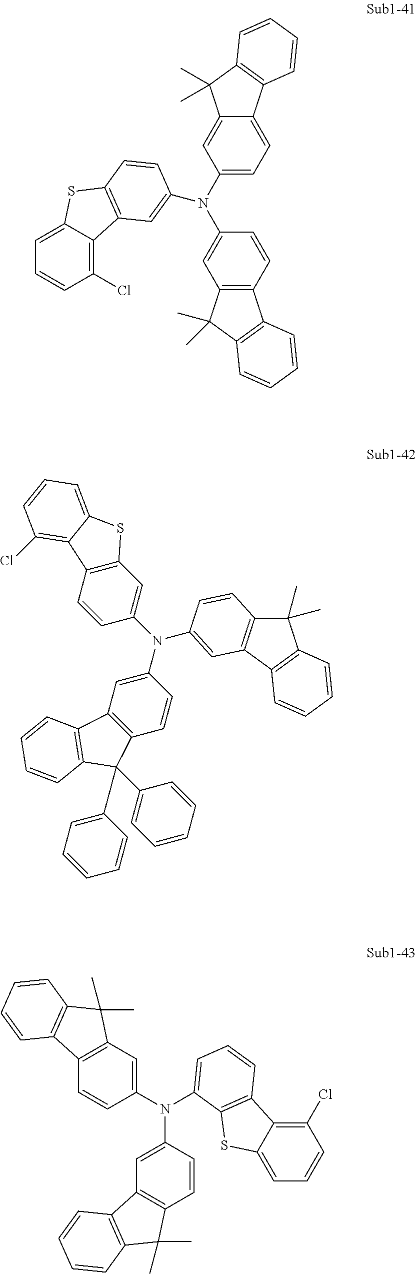 [SMILES: CC1(C)c2ccccc2-c2ccc(N(c3ccc4c(c3)C(C)(C)c3ccccc3-4)c3ccc4sc5cccc(Cl)c5c4c3)cc21 ; CC1(C)c2ccccc2-c2cc(N(c3ccc4c(c3)-c3ccccc3C4(c3ccccc3)c3ccccc3)c3ccc4c(c3)sc3cccc(Cl)c34)ccc21 ; CC1(C)c2ccccc2-c2cc(N(c3ccc4c(c3)C(C)(C)c3ccccc3-4)c3cccc4c3sc3cccc(Cl)c34)ccc21]

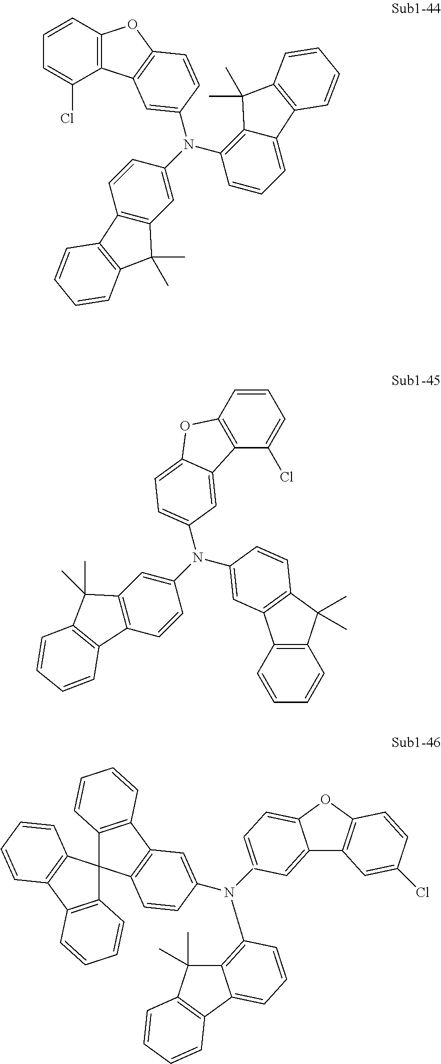

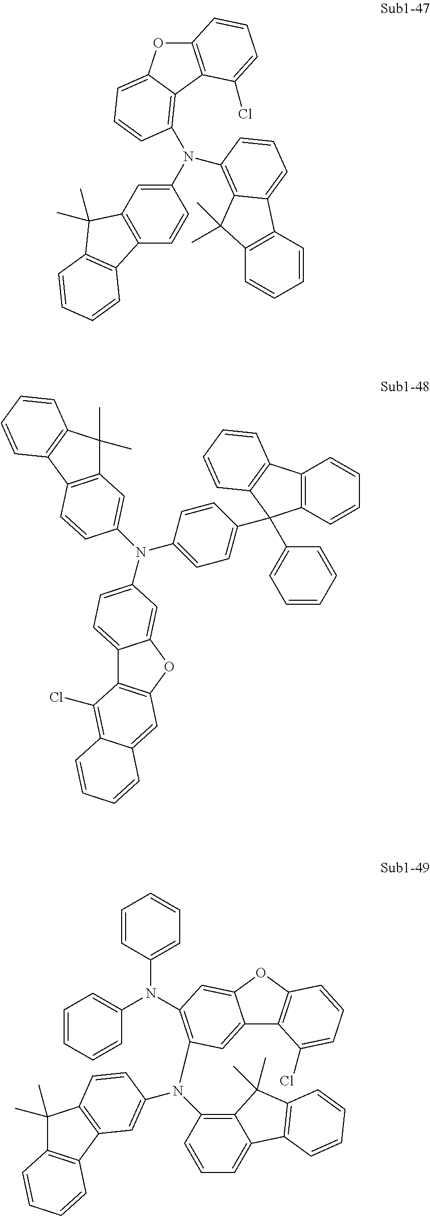 [SMILES: CC1(C)c2ccccc2-c2ccc(N(c3cccc4c3C(C)(C)c3ccccc3-4)c3cccc4oc5cccc(Cl)c5c34)cc21 ; CC1(C)c2ccccc2-c2ccc(N(c3ccc(C4(c5ccccc5)c5ccccc5-c5ccccc54)cc3)c3ccc4c(c3)oc3cc5ccccc5c(Cl)c34)cc21 ; CC1(C)c2ccccc2-c2cc(N(c3cc4c(cc3N(c3ccccc3)c3ccccc3)oc3cccc(Cl)c34)c3cccc4c3C(C)(C)c3ccccc3-4)ccc21]

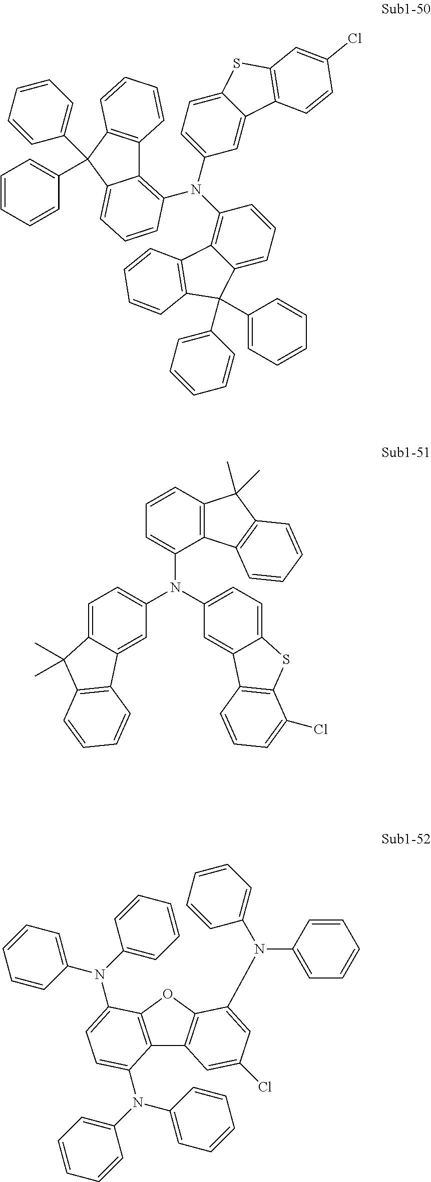 [SMILES: Clc1ccc2c(c1)sc1ccc(N(c3cccc4c3-c3ccccc3C4(c3ccccc3)c3ccccc3)c3cccc4c3-c3ccccc3C4(c3ccccc3)c3ccccc3)cc12 ; CC1(C)c2ccccc2-c2cc(N(c3ccc4sc5c(Cl)cccc5c4c3)c3cccc4c3-c3ccccc3C4(C)C)ccc21 ; Clc1cc(N(c2ccccc2)c2ccccc2)c2oc3c(N(c4ccccc4)c4ccccc4)ccc(N(c4ccccc4)c4ccccc4)c3c2c1]

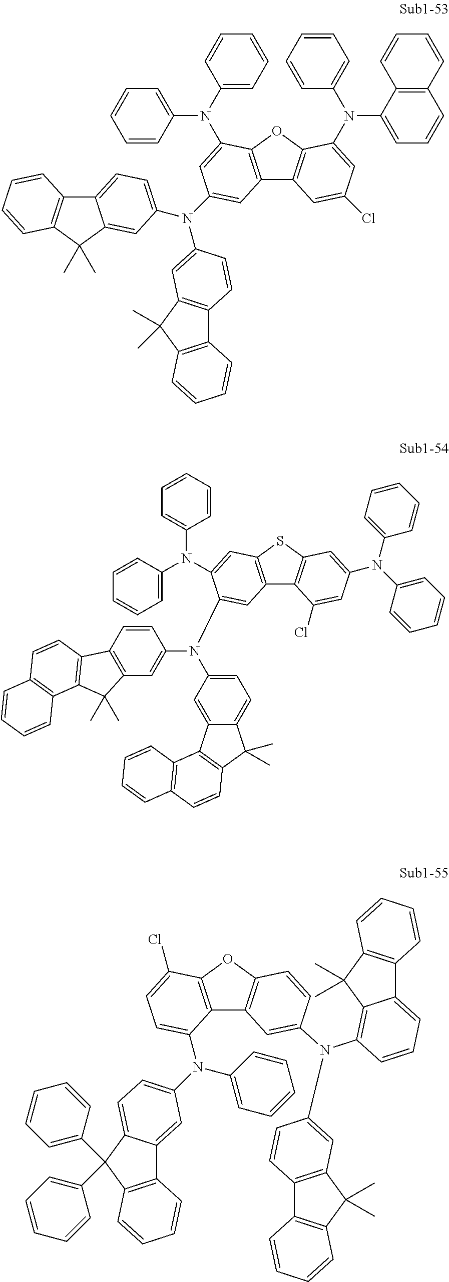 [SMILES: CC1(C)c2ccccc2-c2ccc(N(c3ccc4c(c3)C(C)(C)c3ccccc3-4)c3cc(N(c4ccccc4)c4ccccc4)c4oc5c(N(c6ccccc6)c6cccc7ccccc67)cc(Cl)cc5c4c3)cc21 ; CC1(C)c2ccc(N(c3ccc4c(c3)C(C)(C)c3c-4ccc4ccccc34)c3cc4c(cc3N(c3ccccc3)c3ccccc3)sc3cc(N(c5ccccc5)c5ccccc5)cc(Cl)c34)cc2-c2c1ccc1ccccc21 ; CC1(C)c2ccccc2-c2ccc(N(c3ccc4oc5c(Cl)ccc(N(c6ccccc6)c6ccc7c(c6)-c6ccccc6C7(c6ccccc6)c6ccccc6)c5c4c3)c3cccc4c3C(C)(C)c3ccccc3-4)cc21]

TABLE 1

| compound | FD-MS | compound | FD-MS |
|---|---|---|---|
| Sub1-1 | m/z = 369.09(C₂₄H₁₆CINO = 369.85) | Sub1-2 | m/z = 369.09(C₂₄H₁₆CINO = 369.85) |
| Sub1-3 | m/z = 521.15(C₃₆H₂₄CINO = 522.04) | Sub1-4 | m/z = 446.12(C₂₉H₁₉CIN₂O = 446.93) |
| Sub1-5 | m/z = 571.1 7(C₄₀H₂₆CINO = 572.1) | Sub1-6 | m/z = 613.16(C₄₂H₂₈CINS = 614.2) |
| Sub1-7 | m/z = 485.1 (C₃₂H₂₀CINS = 486.03) | Sub1-8 | m/z = 669.14(C₄₄H₂₈CINS₂ = 670.29) |
| Sub1-9 | m/z = 385.07(C₂₄H₁₆CINS = 385.91) | Sub1-10 | m/z = 369.09(C₂₄H₁₆CINO = 369.85) |
| Sub1-11 | m/z = 537.13(C₃₆H₂₄CINS = 538.11) | Sub1-12 | m/z = 419.08(C₂6Hi4CIN₃O = 419.87) |
| Sub1-13 | m/z = 513.19(C₃₅H₂₈CINO = 514.07) | Sub1-14 | m/z = 773.25(C56H₃6CINO = 774.36) |
| Sub1-15 | m/z = 495.14(C₃₄H₂₂CINO = 496.01) | Sub1-16 | m/z = 511.12(C34H₂2CINS = 512.07) |
| Sub1-17 | m/z = 419.11 (C₂₈H₁₈CINO = 419.91) | Sub1-18 | m/z = 561.13(C38H₂4CINS = 562.13) |
| Sub1-19 | m/z = 552.14(C₃₆H₂₅CIN₂S = 553.12) | Sub1-20 | m/z = 552.14(C₃₆H₂₅CIN₂S = 553.12) |
| Sub1-21 | m/z = 485.15(C₃₃H₂₄CINO = 486.01) | Sub1-22 | m/z = 419.11 (C₂₈H₁₈CINO = 419.91) |
| Sub1-23 | m/z = 617.19(C₄₂H₃₂CINS = 618.24) | Sub1-24 | m/z = 741 23(C₅₂H₃₆CINS = 742.38) |
| Sub1-25 | m/z = 801,23(C₅₇H₃₆CINS = 802.43) | Sub1-26 | m/z = 667.21 (C₄₆H₃₄CINS = 668.3) |
| Sub1-27 | m/z = 667.21 (C₄₆H₃₄CINS = 668.3) | Sub1-28 | m/z = 739.21 (C₅₂H₃₄CINS = 740.36) |
| Sub1-29 | m/z = 823.26(C₆₀H₃₈CINO = 824.42) | Sub1-30 | m/z = 927.33(C₆₈H₄₆CINO = 928.57) |
| Sub1-31 | m/z = 601.22(C₄₂H₃₂CINO = 602.17) | Sub1-32 | m/z = 844.32(C₆₀H₄₅CIN₂0 = 845.48) |
| Sub1-33 | m/z = 1001.34(C₇₄H₄₈CINO = 1002.65) | Sub1-34 | m/z = 601.22(C₄₂H₃₂CINO = 602.17) |
| Sub1-35 | m/z = 601.22(C₄₂H₃₂CINO = 602.17) | Sub1-36 | m/z = 601.22(C₄₂H₃₂CINO = 602.17) |
| Sub1-37 | m/z = 717.23(C₅₀H₃₆CINS = 718.36) | Sub1-38 | m/z = 791,24(C₅₆H₃₈CINS = 792.44) |
| Sub1-39 | m/z = 958.31 (C₆₈H₄₇CIN₂S = 959.65) | Sub1-40 | m/z = 617.19(C₄₂H₃₂CINS = 618.24) |
| Sub1-41 | m/z = 617.19(C₄₂H₃₂CINS = 618.24) | Sub1-42 | m/z = 741,23(C₅₂H₃₆CINS = 742.38) |
| Sub1-43 | m/z = 617.19(C₄₂H₃₂CINS = 618.24) | Sub1-44 | m/z = 601.22(C₄₂H₃₂CINO = 602.17) |
| Sub1-45 | m/z = 601.22(C₄₂H₃₂CINO = 602.17) | Sub1-46 | m/z = 723.23(C₅₂H₃₄CINO = 724.3) |
| Sub1-47 | m/z = 601.22(C₄₂H₃₂CINO = 602.17) | Sub1-48 | m/z = 775.26(C₅₆H₃₈CINO = 776.38) |
| Sub1-49 | m/z = 768.29(C₅₄H₄₁CIN₂O = 769.39) | Sub1-50 | m/z = 865.26(C₆₂H₄₀CINS = 866.52) |
| Sub1-51 | m/z = 617.19(C₄₂H₃₂CINS = 618.24) | Sub1-52 | m/z = 703.24(C₄₈H₃₄CIN₃O = 704.27) |
| Sub1-53 | m/z = 985.38(C₇₀H₅₂CIN₃O = 986.66) | Sub1-54 | m/z = 1051.37(C₇₄H₅₄CIN₃S = 1052.78) |
| Sub1-55 | m/z = 1008.38(C₇₃H₅₃CIN₂O = 1009.69) | | |

Meanwhile, the compound belonging to Sub 2 may be the following compounds, but is not limited thereto, and Table 2 below shows FD-MS values of the compounds belonging to Sub 2.

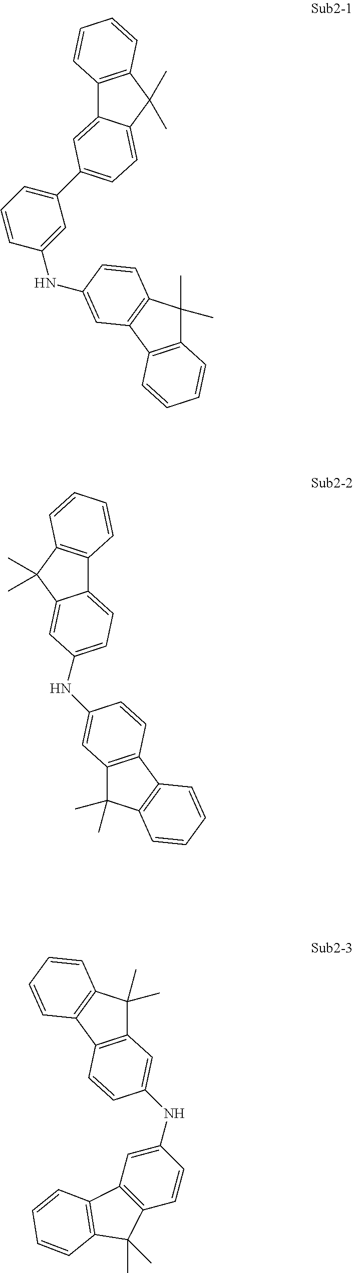

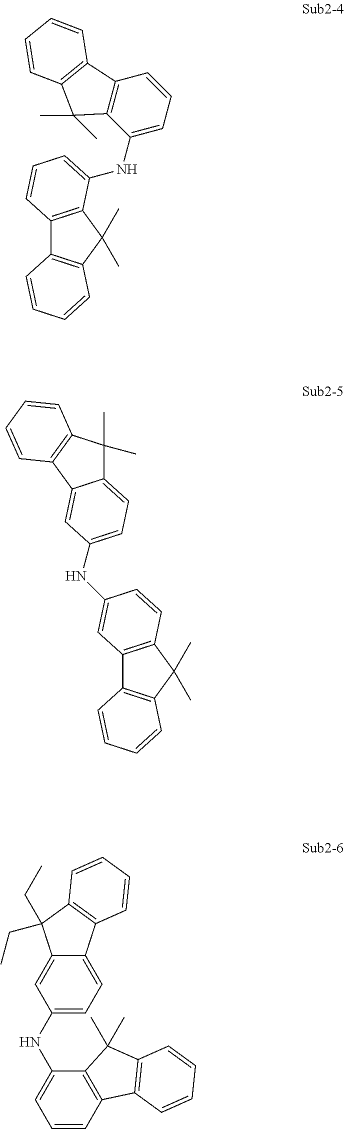

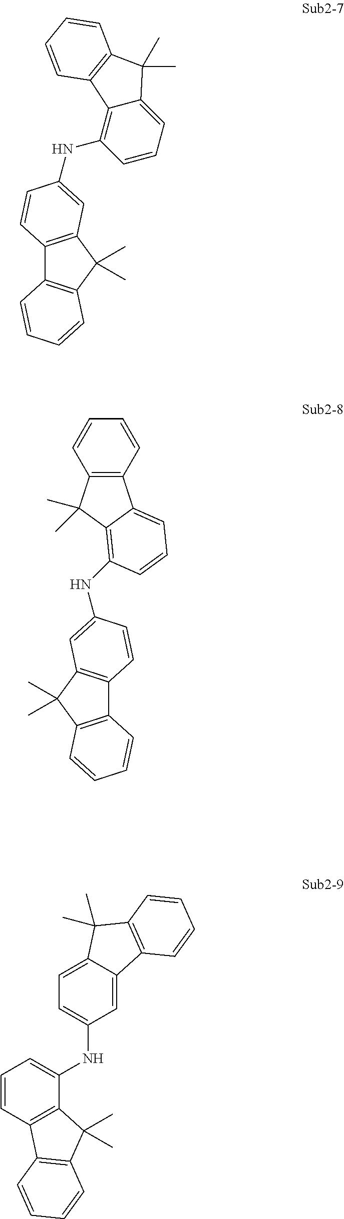

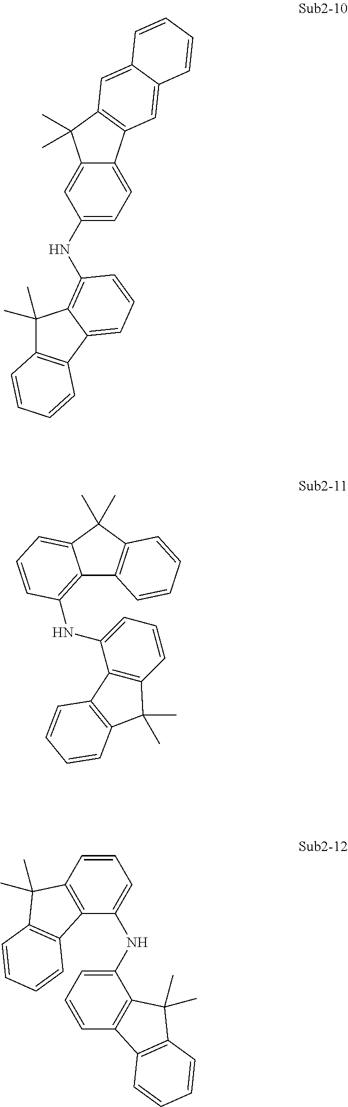

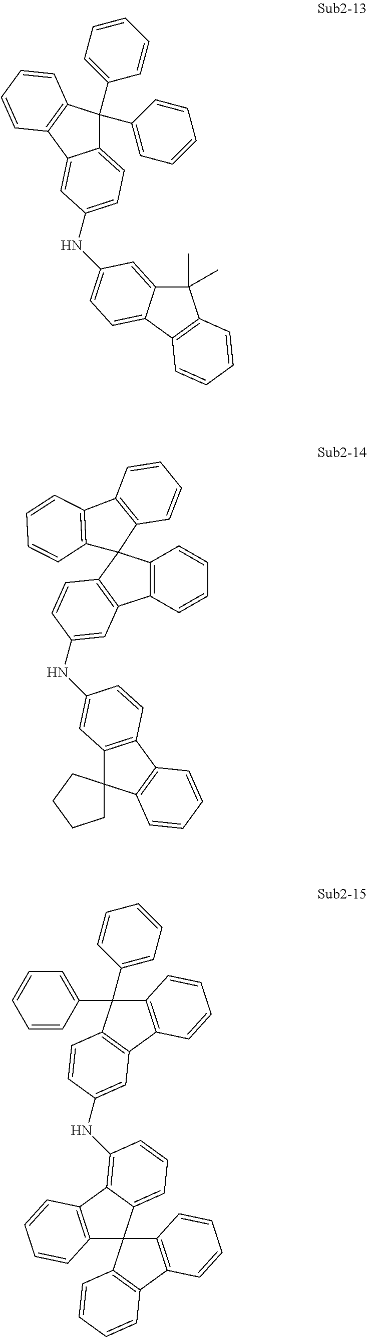 [SMILES: CC1(C)c2ccccc2-c2ccc(Nc3ccc4c(c3)-c3ccccc3C4(c3ccccc3)c3ccccc3)cc21 ; c1ccc2c(c1)-c1ccc(Nc3ccc4c(c3)-c3ccccc3C43c4ccccc4-c4ccccc43)cc1C21CCCC1 ; c1ccc(C2(c3ccccc3)c3ccccc3-c3cc(Nc4cccc5c4-c4ccccc4C54c5ccccc5-c5ccccc54)ccc32)cc1]

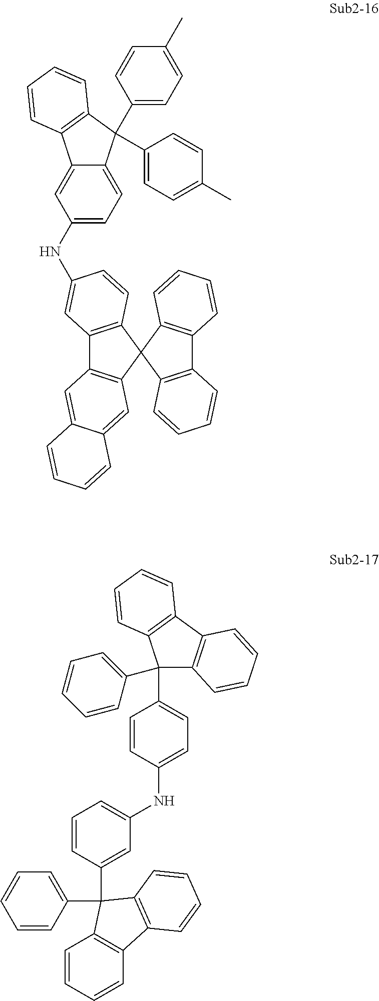 [SMILES: Cc1ccc(C2(c3ccc(C)cc3)c3ccccc3-c3cc(Nc4ccc5c(c4)-c4cc6ccccc6cc4C54c5ccccc5-c5ccccc54)ccc32)cc1 ; c1ccc(C2(c3ccc(Nc4cccc(C5(c6ccccc6)c6ccccc6-c6ccccc65)c4)cc3)c3ccccc3-c3ccccc32)cc1]

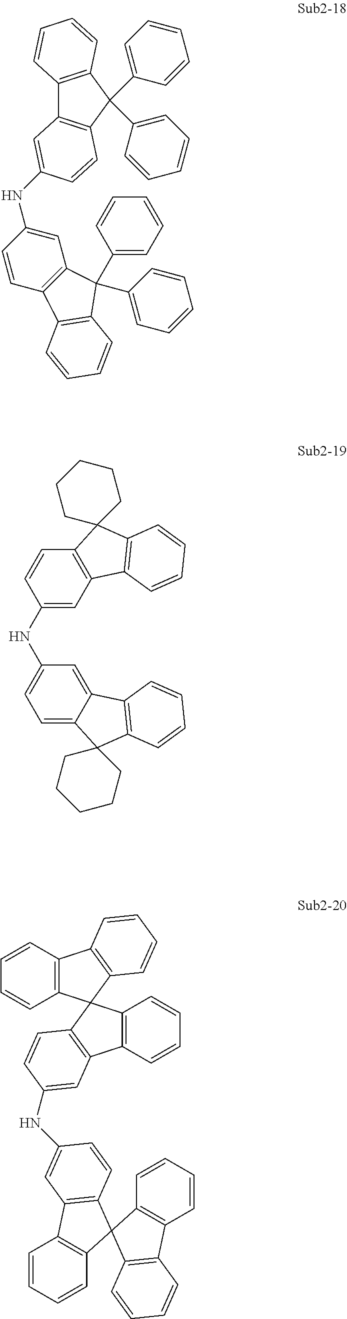 [SMILES: c1ccc(C2(c3ccccc3)c3ccccc3-c3cc(Nc4ccc5c(c4)C(c4ccccc4)(c4ccccc4)c4ccccc4-5)ccc32)cc1 ; c1ccc2c(c1)-c1cc(Nc3ccc4c(c3)-c3ccccc3C43CCCCC3)ccc1C21CCCCC1 ; c1ccc2c(c1)-c1ccccc1C21c2ccccc2-c2cc(Nc3ccc4c(c3)-c3ccccc3C43c4ccccc4-c4ccccc43)ccc21]

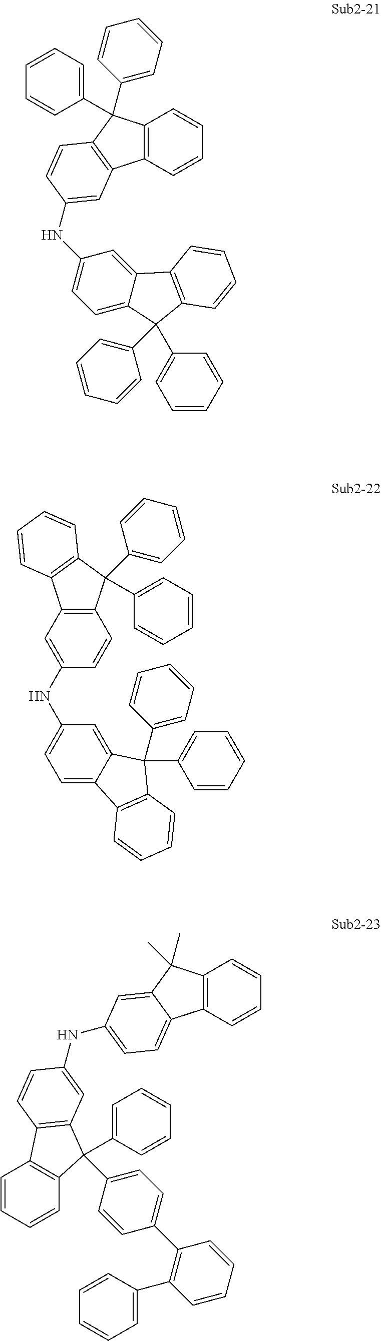 [SMILES: c1ccc(C2(c3ccccc3)c3ccccc3-c3cc(Nc4ccc5c(c4)-c4ccccc4C5(c4ccccc4)c4ccccc4)ccc32)cc1 ; c1ccc(C2(c3ccccc3)c3ccccc3-c3cc(Nc4ccc5c(c4)C(c4ccccc4)(c4ccccc4)c4ccccc4-5)ccc32)cc1 ; CC1(C)c2ccccc2-c2ccc(Nc3ccc4c(c3)C(c3ccccc3)(c3ccc(-c5ccccc5-c5ccccc5)cc3)c3ccccc3-4)cc21]

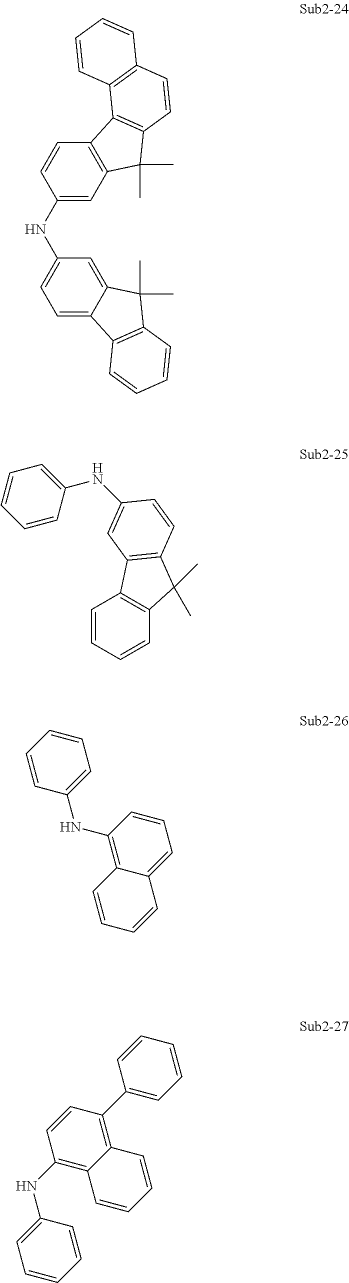 [SMILES: CC1(C)c2ccccc2-c2ccc(Nc3ccc4c(c3)C(C)(C)c3ccc5ccccc5c3-4)cc21 ; CC1(C)c2ccccc2-c2cc(Nc3ccccc3)ccc21 ; c1ccc(Nc2cccc3ccccc23)cc1 ; c1ccc(Nc2ccc(-c3ccccc3)c3ccccc23)cc1]

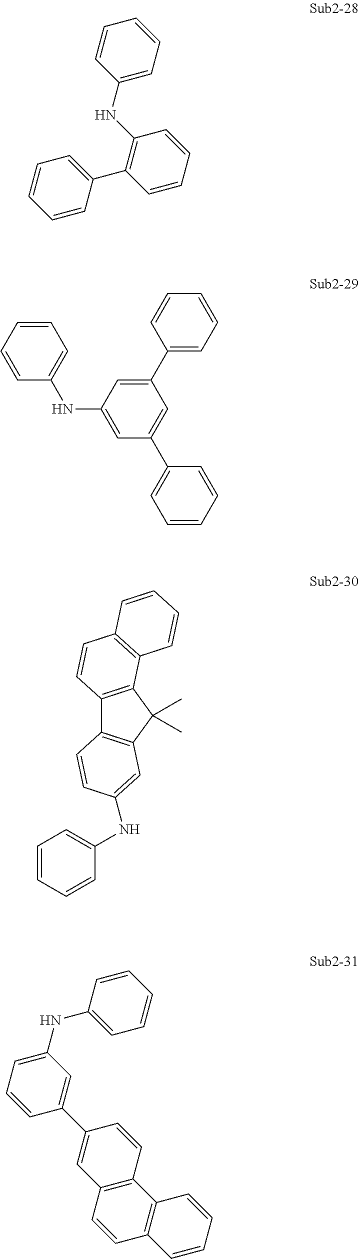 [SMILES: c1ccc(Nc2ccccc2-c2ccccc2)cc1 ; c1ccc(Nc2cc(-c3ccccc3)cc(-c3ccccc3)c2)cc1 ; CC1(C)c2cc(Nc3ccccc3)ccc2-c2ccc3ccccc3c21 ; c1ccc(Nc2cccc(-c3ccc4c(ccc5ccccc54)c3)c2)cc1]

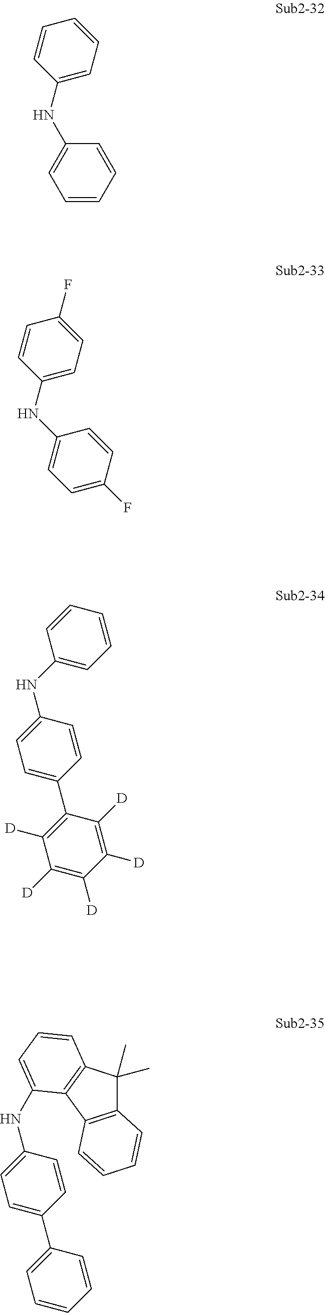 [SMILES: c1ccc(Nc2ccccc2)cc1 ; Fc1ccc(Nc2ccc(F)cc2)cc1 ; [2H]c1c([2H])c([2H])c(-c2ccc(Nc3ccccc3)cc2)c([2H])c1[2H] ; CC1(C)c2ccccc2-c2c(Nc3ccc(-c4ccccc4)cc3)cccc21]

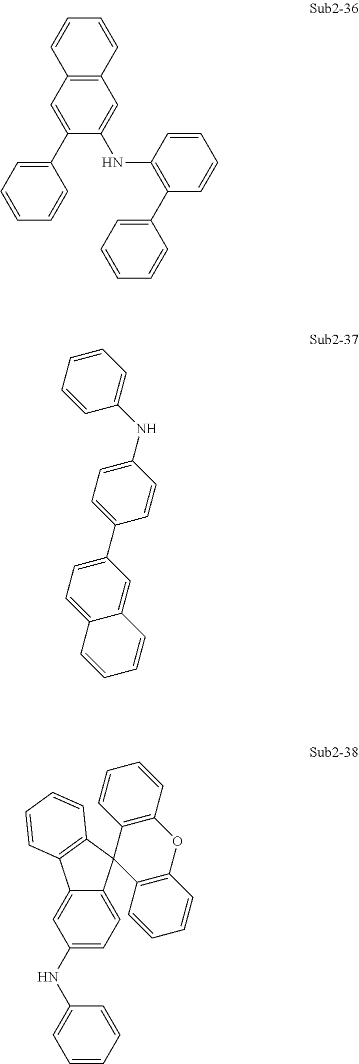 [SMILES: c1ccc(-c2ccccc2Nc2cc3ccccc3cc2-c2ccccc2)cc1 ; c1ccc(Nc2ccc(-c3ccc4ccccc4c3)cc2)cc1 ; c1ccc(Nc2ccc3c(c2)-c2ccccc2C32c3ccccc3Oc3ccccc32)cc1]

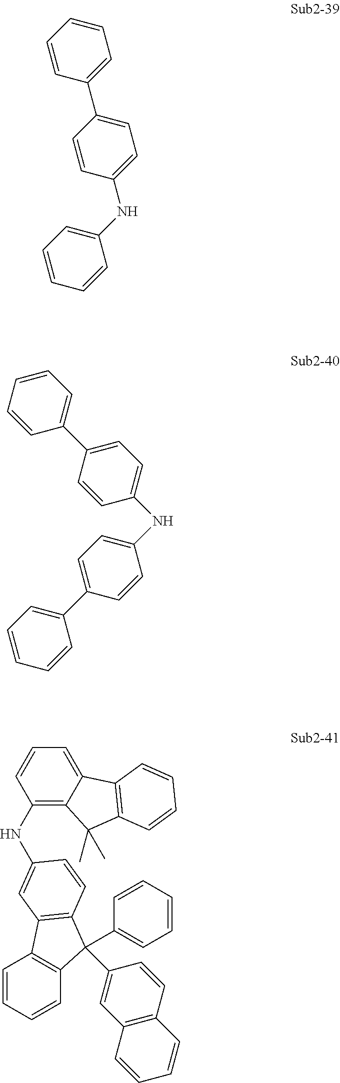 [SMILES: c1ccc(Nc2ccc(-c3ccccc3)cc2)cc1 ; c1ccc(-c2ccc(Nc3ccc(-c4ccccc4)cc3)cc2)cc1 ; CC1(C)c2ccccc2-c2cccc(Nc3ccc4c(c3)-c3ccccc3C4(c3ccccc3)c3ccc4ccccc4c3)c21]

TABLE 2

| compound | FD-MS | compound | FD-MS |
|---|---|---|---|
| Sub2-1 | m/z = 477.25(C₃₆H₃₁N = 477.65) | Sub2-2 | m/z = 401.21(C₃oH27N = 401.55) |
| Sub2-3 | m/z = 401.21(C₃₀H₂₇N = 401.55) | Sub2-4 | m/z = 401.21(C₃₀H₂₇N = 401.55) |
| Sub2-5 | m/z = 401.21(C₃₀H₂₇N = 401.55) | Sub2-6 | m/z = 429.25(C₃₂H₃₁N = 429.61) |
| Sub2-7 | m/z = 401.21(C₃₀H₂₇N = 401.55) | Sub2-8 | m/z = 401.21(C₃₀H₂₇N = 401.55) |
| Sub2-9 | m/z = 401.21(C₃₀H₂₇N = 401.55) | Sub2-10 | m/z = 45123(C₃₄H₂₉N = 451.61) |
| Sub2-11 | m/z = 401.21(C₃₀H₂₇N = 401.55) | Sub2-12 | m/z = 401.21(C₃₀H₂₇N = 401.55) |
| Sub2-13 | m/z = 525.25(C₄₀H₃₁N = 525.7) | Sub2-14 | m/z = 549.25(C₄₂H₃₁N = 549.72) |
| Sub2-15 | m/z = 647.26(C₅₀H₃₃N = 647.82) | Sub2-16 | m/z = 725.31(C₅₆H₃₉N = 725.94) |
| Sub2-17 | m/z = 649.28(C₅₀H₃₅N = 649.84) | Sub2-18 | m/z = 649.28(C₅₀H₃₅N = 649.84) |
| Sub2-19 | m/z = 48128(C₃₆H₃₅N = 481.68) | Sub2-20 | m/z = 645.25(C₅0H₃₁N = 645.81) |
| Sub2-21 | m/z = 649.28(C₅₀H35N = 649.84) | Sub2-22 | m/z = 649.28(C₅₀H₃₅N = 649.84) |
| Sub2-23 | m/z = 677.31(C₅₂H₃₉N = 677.89) | Sub2-24 | m/z = 451.23(C₃₄H₂₉N = 451.61) |
| Sub2-25 | m/z = 285.15(C₂H₁₉N = 285.39) | Sub2-26 | m/z = 219.1(C₁₆H₁₃N = 219.29) |
| Sub2-27 | m/z = 295.14(C₂₂H₁₇N = 295.39) | Sub2-28 | m/z = 245.12(C₁₈H₁₅N = 245.33) |
| Sub2-29 | m/z = 321.15(C₂₄H₁₉N = 321.42) | Sub2-30 | m/z = 335.17(C₂₅H₂₁N = 335.45) |
| Sub2-31 | m/z = 345.15(C₂₆H19N = 345.45) | Sub2-32 | m/z = 169.09(C₁₂H₁₁N = 169.23) |
| Sub2-33 | m/z = 205.07(C₁₂H₉F₂N = 205.21) | Sub2-34 | m/z = 250.15(C₁₈H₁₀D₅N = 250.36) |
| Sub2-35 | m/z = 361.18(C₂₇H₂₃N = 361.49) | Sub2-36 | m/z = 371.17(C₂₈H₂₁N = 371.48) |
| Sub2-37 | m/z = 295.14(C₂₂H₁₇N = 295.39) | Sub2-38 | m/z = 423.16(C₃₁H₂₁NO = 423.52) |
| Sub2-39 | m/z = 245.12(C₁₈H₁₅N = 245.33) | Sub2-40 | m/z = 321.15(C₂₄H₁₉N = 321.42) |
| Sub2-41 | m/z = 575.26(C₄₄H₃₃N = 575.76) | | |

### II. Synthesis of Product

### 1. Synthesis Example of P-2

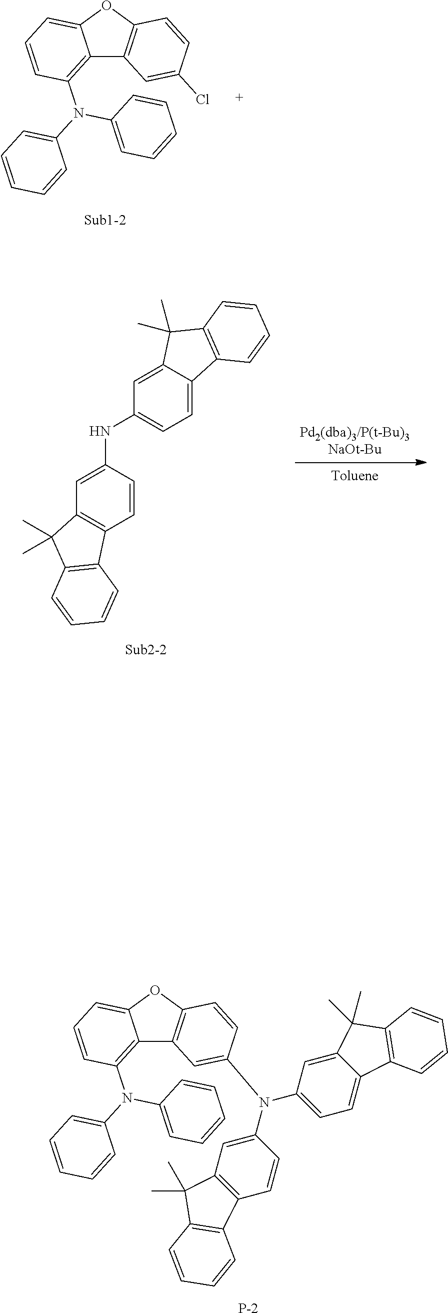

Sub 1-2 (30 g, 0.08 mol) and Sub 2-2 (32.5 g, 0.08 mol), Pd₂(dba)3 (2.2 g, 0.0024 mol), 50% P(t-Bu)₃ (2.0 g, 0.005 mol), NaOt-Bu (23.4 g, 0.24 mol), toluene (150 ml) were added to a round flask and stirred at 124° C. When the reaction was completed, 50 g of product P-2 (yield: 84%) was obtained by concentrating the reaction product and then recrystallizing the resulting compound through a silicagel column and recrystallization method.

### 2. Synthesis Example of P-9

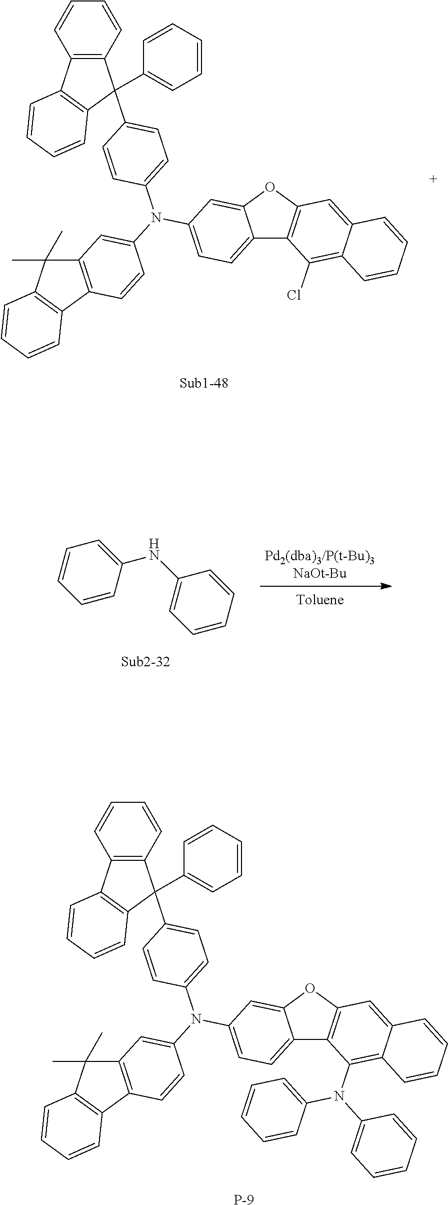

Sub 1-48 (50 g, 0.06 mol) and Sub 2-32 (10.9 g, 0.06 mol), Pd₂(dba)₃ (1.8 g, 0.0019 mol), 50% P(t-Bu)3 (1.6 g, 0.004 mol), NaOt-Bu (18.6 g, 0.19 mol), toluene (130 ml) were added to a round flask and stirred at 124° C. When the reaction was completed, 48 g (yield: 82%) of the product P-9 was obtained through the separation method of P-1.

### 3. Synthesis Example of P-20

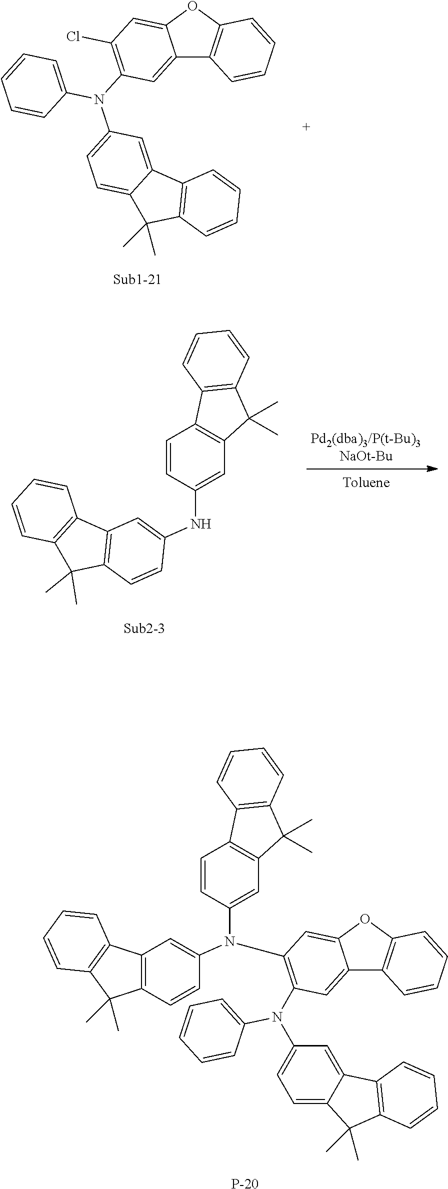

Sub 1-21 (50 g, 0.10 mol) and Sub 2-3 (41.3 g, 0.10 mol), Pd₂(dba)₃ (2.8 g, 0.003 mol), 50% P(t-Bu)₃ (2.5 g, 0.006 mol), NaOt-Bu (30 g, 0.31 mol), toluene (160 ml) were added to a round flask and stirred at 124° C. When the reaction was completed, 80 g (yield: 91.4%) of the product P-20 was obtained through the separation method of P-1.

### 4. Synthesis Example of P-26

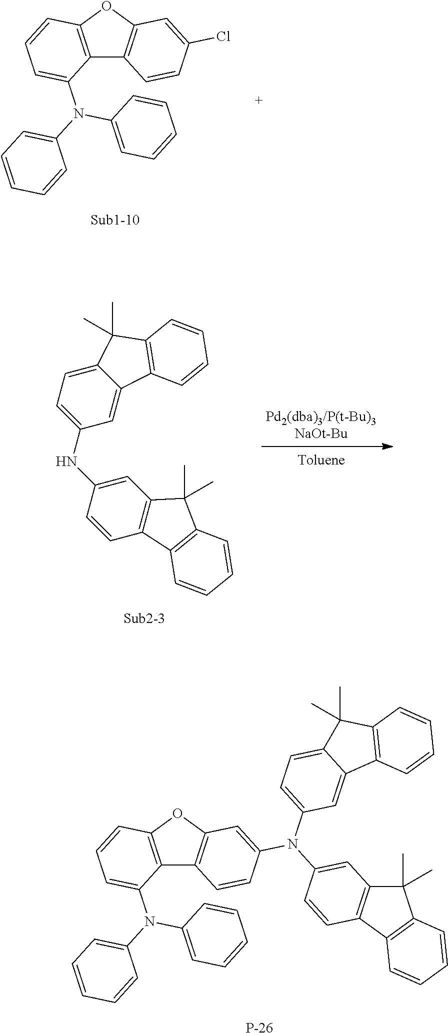

Sub 1-10 (50 g, 0.14 mol) and Sub 2-3 (54.2 g, 0.14 mol), Pd₂(dba)₃ (3.7 g, 0.004 mol), 50% P(t-Bu)₃ (3.3 g, 0.008 mol), NaOt-Bu (39 g, 0.41 mol), toluene (200 ml) were added to a round flask and stirred at 124° C. When the reaction was completed, 80 g (yield: 80.6%) of the product P-26 was obtained through the separation method of P-1.

### 5. Synthesis Example of P-29

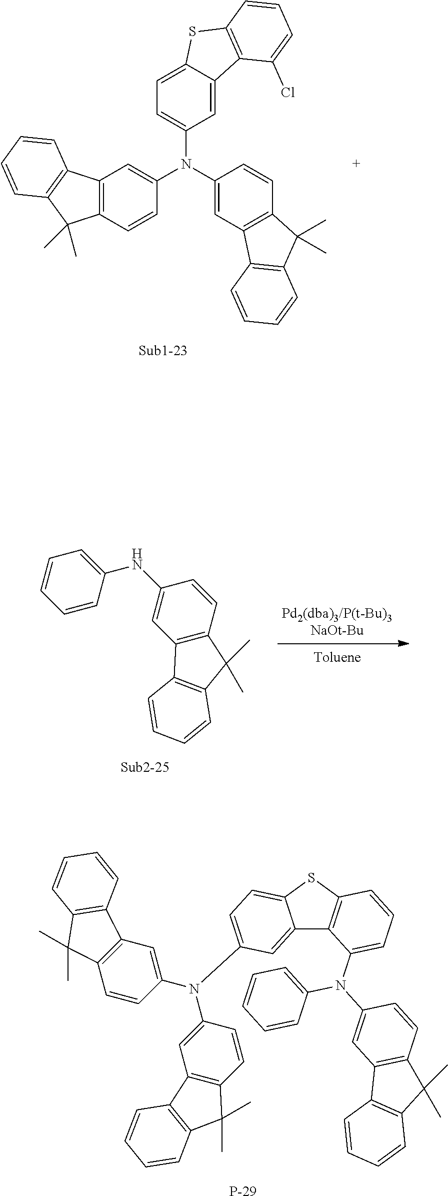

Sub 1-23 (35 g, 0.06 mol) and Sub 2-25 (16.2 g, 0.06 mol), Pd₂(dba)₃ (1.6 g, 0.002 mol), 50% P(t-Bu)₃ (1.4 g, 0.004 mol), NaOt-Bu (16.3 g, 0.17 mol), toluene (130 ml) were added to a round flask and stirred at 124° C. When the reaction was completed, 40 g (yield: 81.5%) of the product P-29 was obtained through the separation method of P-1.

### 6. Synthesis Example of P-34

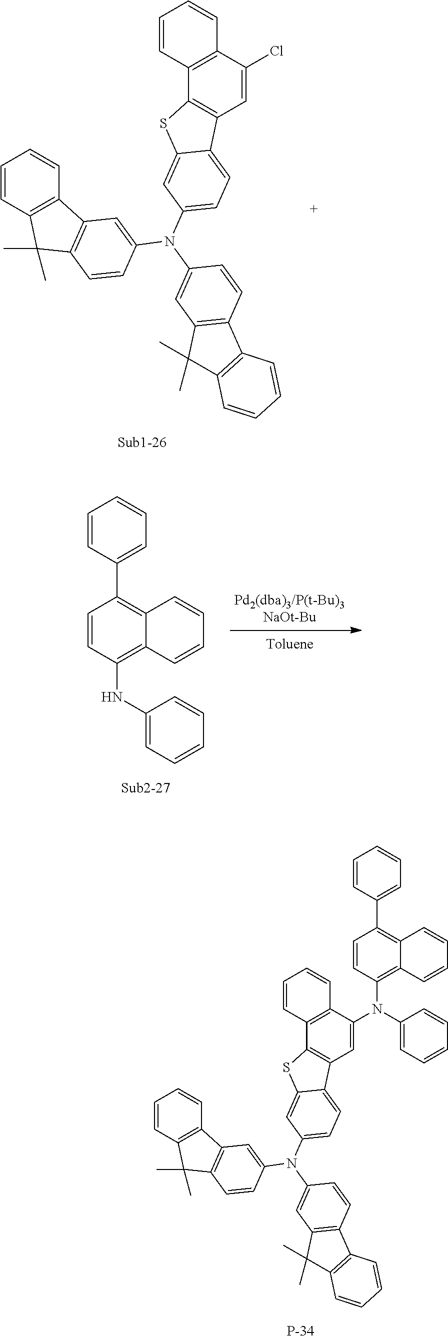

Sub 1-26 (35 g, 0.05 mol) and Sub 2-27 (15.5 g, 0.06 mol), Pd₂(dba)₃ (1.4 g, 0.002 mol), 50% P(t-Bu)₃ (1.3 g, 0.004 mol), NaOt-Bu (15.1 g, 0.16 mol), toluene (100 ml) were added to a round flask and stirred at 124° C. When the reaction was completed, 41 g (yield: 84.4%) of the product P-34 was obtained through the separation method of P-1.

### 7. Synthesis Example of P-42

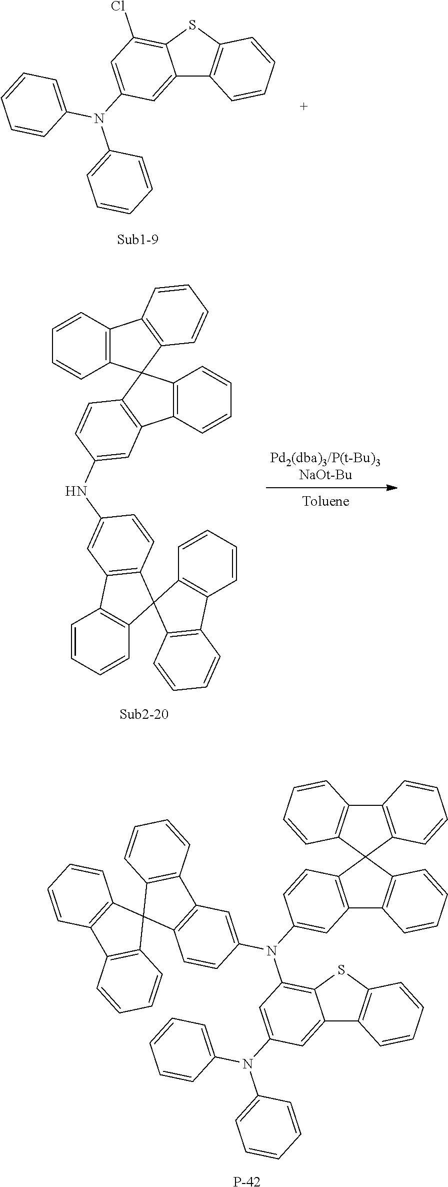

Sub 1-9 (40 g, 0.10 mol) and Sub 2-20 (66.9 g, 0.10 mol), Pd₂(dba)₃ (2.8 g, 0.003 mol), 50% P(t-Bu)₃ (2.5 g, 0.006 mol), NaOt-Bu (29.9 g, 0.31 mol), toluene (180 ml) were added to a round flask and stirred at 124° C. When the reaction was completed, 87 g (yield: 84.3%) of the product P-42 was obtained through the separation method of P-1.

### 8. Synthesis Example of P-47

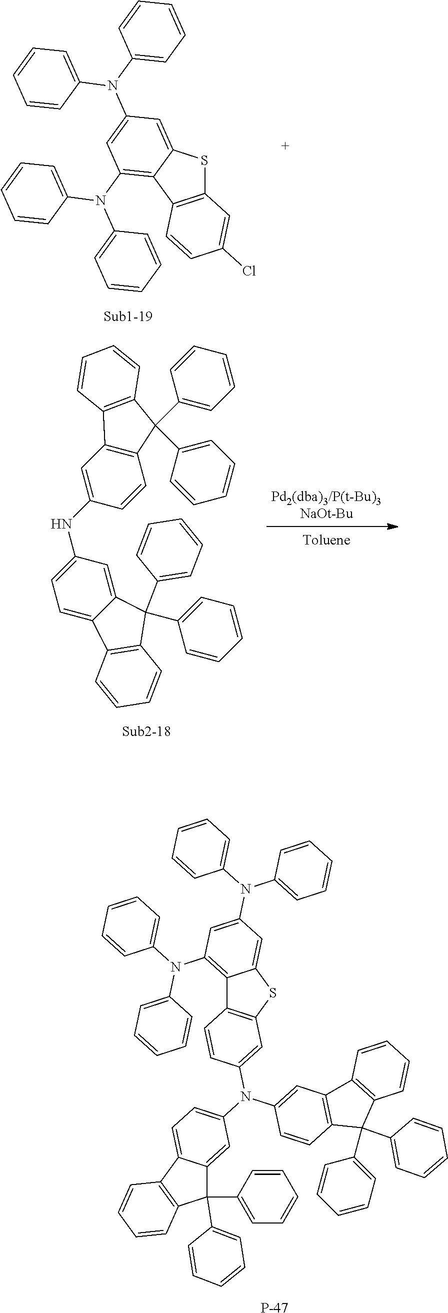

Sub 1-19 (38 g, 0.07 mol) and Sub 2-18 (44.6 g, 0.07 mol), Pd₂(dba)₃ (1.9 g, 0.002 mol), 50% P(t-Bu)₃ (1.7 g, 0.004 mol), NaOt-Bu (19.8 g, 0.21 mol), toluene (160 ml) were added to a round flask and stirred at 124° C. When the reaction was completed, 72 g (yield: 89.8%) of the product P-47 was obtained through the separation method of P-1.

### 9. Synthesis Example of P-58

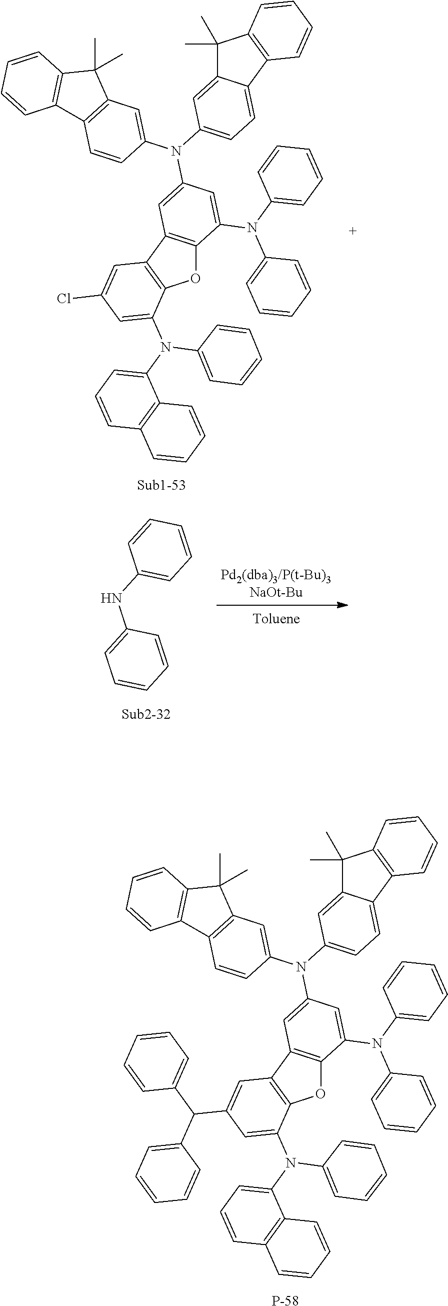

Sub 1-53 (50 g, 0.05 mol) and Sub 2-32 (8.6 g, 0.05 mol), Pd₂(dba)₃ (1.4 g, 0.002 mol), 50% P(t-Bu)₃ (1.2 g, 0.003 mol), NaOt-Bu (14.6 g, 0.15 mol), toluene (100 ml) were added to a round flask and stirred at 124° C. When the reaction was completed, 50 g (yield: 88.1%) of the product P-58 was obtained through the separation method of P-1.

Meanwhile, the FD-MS values of the compounds P-1 to P-60 of the present invention prepared according to the above synthesis examples are shown in Table 3.

TABLE 3

| compound | FD-MS | compound | FD-MS |
|---|---|---|---|
| P-1 | m/z = 910.39(C₆₈H₅₀N₂O = 911.16) | P-2 | m/z = 734.33(C54H₄₂N₂O = 734.94) |
| P-3 | m/z = 1010.42(C₇₆H₅₄N₂O = 1011.28) | P-4 | m/z = 959.39(C₇₁H₄₉N₃O = 960.19) |
| P-5 | m/z = 734.33(C₅₄H₄₂N₂O = 734.94) | P-6 | m/z = 892.33(C₆₄H₄₂F₂N₂O = 893.05) |
| P-7 | m/z = 734.33(C₅₄H₄₂N₂O = 734.94) | P-8 | m/z = 1030.39(C₇₈H₅₀N₂O = 1031.27) |
| P-9 | m/z = 908.38(C₆₈H₄₈N₂O = 909.15) | P-10 | m/z = 1215.52(C₉₂H₅₇D₅N₂O = 1216.55) |
| P-11 | m/z = 734.33(C₅₄H₄₂N₂O = 734.94) | P-12 | m/z = 1012.44(C₇₆H₅₆N₂O = 1013.3) |
| P-13 | m/z = 926.42(C₆₉H₅₄N₂O = 927.2) | P-14 | m/z = 936.41(C₇₀H₅₂N₂O = 937.2) |
| P-15 | m/z = 1032.38(C₇₆H₄₈N₄O = 1033.25) | P-16 | m/z = 878.42(C₆₅H₅₄N₂O = 879.16) |
| P-17 | m/z = 1166.52(C₈₈H₆N₂O = 1167.51) | P-18 | m/z = 1184.47(C₉₀H₆₀N₂O = 1185.48) |
| P-19 | m/z = 1032.41(C₇₈H₅₂N₂O = 1033.29) | P-20 | m/z = 850.39(C₆₃H₅₀N₂O = 851.1 |
| P-21 | m/z = 956.38(C₇₂H48N₂O = 957.19) | P-22 | m/z = 1060.44(C₈₀H₅₆N₂O = 1061.34) |
| P-23 | m/z = 1231.51(C₉₁H₆₅N₃O₂ = 1232.54) | P-24 | m/z = 1027.45(C₇₆H₅₇N₃O = 1028.31) |
| P-25 | m/z = 734.33(C₅₄H₄₂N₂O = 734.94) | P-26 | m/z = 734.33(C₅₄H₄₂N₂O = 734.94) |
| P-27 | m/z = 734.33(C₅₄H₄₂N₂O = 734.94) | P-28 | m/z = 734.33(C₅₄H₄₂N₂O = 734.94) |
| P-29 | m/z = 866.37(C₆₃H₅₀N₂S = 867.17) | P-30 | m/z = 874.34(C₆₄H₄₆N₂S = 875.15) |
| P-31 | m/z = 1048.39(C₇₈H₅₂N₂S = 1049.35) | P-32 | m/z = 982.43(C₇₂H₅₈N₂S = 983.33) |
| P-33 | m/z = 934.34(C₆₉H₄₆N₂S = 935.2) | P-34 | m/z = 926.37(C₆₈H₅₀N₂S = 927.22) |
| P-35 | m/z = 1082.46(C₈₀H₆₂N₂S = 1083.45) | P-36 | m/z = 1254.49(C₉₄H₆₆N₂S = 1255.64) |
| P-37 | m/z = 850.34(C₆₂H₄₆N₂S = 851.12) | P-38 | m/z = 1114.44(C₈₀H₆₂N₂S₂ = 1115.51) |
| P-39 | m/z = 948.35(C₇₀H₄₈N₂S = 949.23) | P-40 | m/z = 1002.4(C₇₄H₅4N₂S = 1003.32) |
| P-41 | m/z = 924.35(C₆₈H₄₈N₂S = 925.21) | P-42 | m/z = 994.34(C₇₄H₄6N₂S = 995.26) |
| P-43 | m/z = 1150.43(C₈₆H₅₈N₂S = 1151.48) | P-44 | m/z = 1257.51(C₉₃H₆₇N₃S = 1258.64) |
| P-45 | m/z = 926.37(C₆₈H₅₀N₂S = 927.22) | P-46 | m/z = 926.37(C₆₈H₅₀N₂S = 927.22) |
| P-47 | m/z = 1165.44(C₈₆H₅₉N₃S = 1166.5) | P-48 | m/z = 1091.43(C₈₀H₅₇N₃S = 1092.42) |
| P-49 | m/z = 750.31(C₅₄H₄₂N₂S = 751) | P-50 | m/z = 750.31(C₅₄H₄₂N₂S = 751) |
| P-51 | m/z = 950.37(C₇₀H₅₀N₂S = 951.24) | P-52 | m/z = 902.37(C₆₆H₅₀N₂S = 903.2) |
| P-53 | m/z = 734.33(C₅₄H₄₂N₂O = 734.94) | P-54 | m/z = 988.4(C₇₃H₅₂N₂O₂ = 989.23) |
| P-55 | m/z = 1129.44(C₈₃H₅₉N₃S = 1130.47) | P-56 | m/z = 1156.44(C₈₅H₆₀N₂OS = 1157.49) |
| P-57 | m/z = 1068.48(C₇₈H₆₀N₄₀ = 1069.37) | P-58 | m/z = 1118.49(C₈₂H₆₂N₄O = 1119.43) |
| P-59 | m/z = 1184.49(C₈₆H₆₄N₄S = 1185.55) | P-60 | m/z = 1141.5(C₈₅H₆₃N₃O = 1142.46) |

### Example 1

### Green Organic Light Emitting Device (Emitting Auxiliary Layer)

First, on an ITO layer (anode) formed on a glass substrate, N¹-(naphthalen-2-yl)-N⁴, N⁴-bis(4-(naphthalen-2-yl(phenyl)amino)phenyl)-N¹-phenylbenzene-1,4-diamine (Hereinafter, abbreviated as 2-TNATA) film as a hole injection layer was vacuum-deposited to form a thickness of 60 nm. Subsequently, 4,4-bis[N-(1-naphthyl)-N-phenylamino]biphenyl (hereinafter abbreviated as -NPD) as a hole transport corn pound was vacuum-deposited on the film to a thickness of 60 nm to form a hole transport layer. Then, as a material for the emitting auxiliary layer, the compounds represented by Formula 1 and Comparative Example were vacuum-deposited to a thickness of 20 nm to form an emitting auxiliary layer. After forming the emitting auxiliary layer, CBP[4,4′-N,N′-dicarbazole-biphenyl] was used as a host material, and Ir(ppy)3 [tris(2-phenylpyridine)-iridium] was used as a dopant material, doped at a weight ratio of 95:5, and vacuum deposited to a thickness of 30 nm to form an emitting layer on the emitting auxiliary layer. (1,1′-bisphenyl)-4-oleato)bis(2-methyl-8-quinolineoleato)aluminum (hereinafter abbreviated as BAlq) as a hole blocking layer was vacuum-deposited to a thickness of 10 nm, as an electron transport layer, tris(8-quinolinol)aluminum (hereinafter, abbreviated as Alq3) was deposited to a thickness of 40 nm. Thereafter, LiF, which is an alkali metal halide, was deposited as an electron injection layer to a thickness of 0.2 nm, and then Al was deposited to a thickness of 150 nm and used as a cathode to prepare an organic electroluminescent device.

### Example 2 to Example 17

An organic electroluminescent device was manufactured in the same manner as in Example 1, except that the compound of the present invention described in Table 4 was used instead of the compound P-2 of the present invention as an emitting auxiliary layer material.

### Comparative Example 1

An organic electroluminescent device was manufactured in the same manner as in Example 1, except that the emitting auxiliary layer was not used.

### Comparative Example 2 to Comparative Example 3

An organic electroluminescent device was manufactured in the same manner as in Example 1, except that comparative compounds A to B were used instead of the compound P-2 of the present invention as an emitting auxiliary layer material.

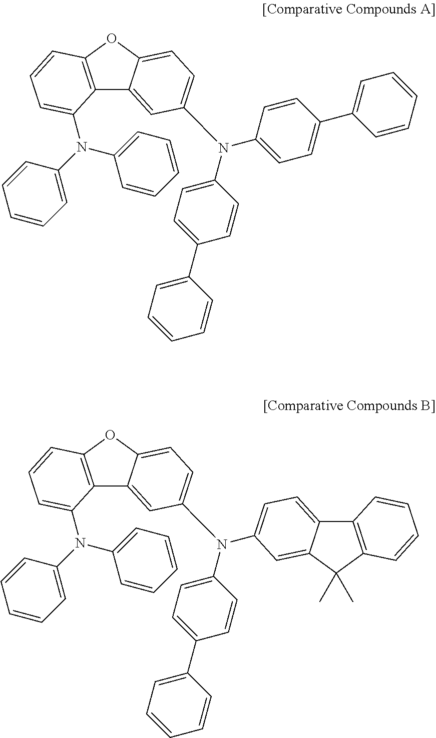 [SMILES: c1ccc(-c2ccc(N(c3ccc(-c4ccccc4)cc3)c3ccc4oc5cccc(N(c6ccccc6)c6ccccc6)c5c4c3)cc2)cc1 ; CC1(C)c2ccccc2-c2ccc(N(c3ccc(-c4ccccc4)cc3)c3ccc4oc5cccc(N(c6ccccc6)c6ccccc6)c5c4c3)cc21]

By applying a forward bias DC voltage to the organic electroluminescent devices prepared in Examples and Comparative Examples prepared in this way, Electroluminescence (EL) characteristics were measured with PR-650 from Photoresearch, and as a result of the measurement, the T95 lifetime was measured using a lifetime measuring device manufactured by McScience at 5000 cd/m² standard luminance. Table 4 below shows the device fabrication and evaluation results.

TABLE 4

| | | | Current | | | | | |
|---|---|---|---|---|---|---|---|---|
| | | | Density | Brightness | Efficiency | | CIE | |
| | compound | Voltage | (mA/cm²) | (cd/m²) | (cd/A) | T(95) | x | y |
| comparative | — | 6.4 | 17.8 | 5000 | 28.1 | 99.4 | 0.34 | 0.61 |
| example(1) | | | | | | | | |
| comparative | comparative | 6.3 | 15.3 | 5000 | 32.7 | 105.9 | 0.34 | 0.64 |
| example(2) | compound | | | | | | | |
| | A | | | | | | | |
| comparative | comparative | 6.0 | 13.7 | 5000 | 36.4 | 114.9 | 0.33 | 0.65 |
| example(3) | compound | | | | | | | |
| | B | | | | | | | |
| example(1) | P-2 | 4.9 | 8.9 | 5000 | 56.2 | 133.0 | 0.33 | 0.65 |
| example(2) | P-5 | 4.9 | 9.0 | 5000 | 55.3 | 132.3 | 0.33 | 0.64 |
| example(3) | P-7 | 5.0 | 9.5 | 5000 | 52.7 | 130.2 | 0.33 | 0.64 |
| example(4) | P-10 | 5.1 | 10.1 | 5000 | 49.3 | 129.5 | 0.33 | 0.64 |
| example(5) | P-20 | 5.3 | 10.3 | 5000 | 48.4 | 128.1 | 0.33 | 0.65 |
| example(6) | P-21 | 5.2 | 10.5 | 5000 | 47.6 | 128.8 | 0.33 | 0.65 |
| example(7) | P-25 | 5.0 | 9.3 | 5000 | 53.6 | 130.9 | 0.33 | 0.65 |
| example(8) | P-26 | 4.9 | 9.2 | 5000 | 54.5 | 131.6 | 0.33 | 0.65 |
| example(9) | P-29 | 4.8 | 9.8 | 5000 | 51.0 | 125.9 | 0.33 | 0.65 |
| example(10) | P-31 | 5.1 | 10.7 | 5000 | 46.7 | 123.8 | 0.33 | 0.65 |
| example(11) | P-35 | 5.0 | 11.8 | 5000 | 42.4 | 122.4 | 0.33 | 0.65 |
| example(12) | P-39 | 5.2 | 10.9 | 5000 | 45.8 | 124.5 | 0.33 | 0.65 |
| example(13) | P-42 | 5.3 | 11.3 | 5000 | 44.1 | 123.1 | 0.33 | 0.65 |
| example(14) | P-47 | 5.3 | 11.6 | 5000 | 43.2 | 121.7 | 0.33 | 0.64 |
| example(15) | P-50 | 4.8 | 9.6 | 5000 | 51.9 | 126.6 | 0.33 | 0.65 |
| example(16) | P-52 | 5.2 | 10.0 | 5000 | 50.1 | 125.2 | 0.33 | 0.65 |
| example(17) | P-57 | 5.4 | 11.1 | 5000 | 45.0 | 127.4 | 0.33 | 0.64 |

As can be seen from the results in Table 4, when manufacturing a green organic electronic element using the material for an organic electronic element of the present invention as an emitting auxiliary layer material, it can be seen that not only can the driving voltage of the organic electroluminescent device be lowered, but also the luminous efficiency and lifespan are remarkably improved compared to Comparative Examples in which no emitting auxiliary layer is used or Comparative Compounds A to B are used. Comparing the results of Comparative Examples 2 to 3, it can be seen that as fluorene is substituted for the amino group, the driving voltage is pulled, the efficiency is increased, and the lifespan is increased. Comparing with the compounds of Examples 1 to 17 corresponding to Formula 1 of the present patent, 2 or more fluorene groups are substituted in the substituents of the amino group. As the fluorene groups are substituted in this way, the overall HOMO level can be raised. When a plurality of fluorenes are substituted, the effect is doubled and the hole injection characteristic from the hole transport layer can be improved. In addition, by substituting a fluorene group with strong hole characteristics for an amine, the stability of the hole is increased, and thus the lifespan of the device as a whole is also increased. And compared with a simple aryl group, the fluorene group has a plate-like structure on the structure, and this plate-like structure improves hole mobility in the overall device and pulls the driving voltage.

Comparing the device results of Examples 1 to 17, the driving voltage, efficiency, and lifespan characteristics are different depending on the type of element included in the linker. In more detail, when the element of the Linker is S, the overall driving voltage and efficiency characteristics are excellent, but it can be seen that the lifespan characteristics are lowered compared to Linker containing the element O. Moreover, it can be seen that different characteristics are shown depending on the bonding position of the amine bonded to Linker, and these characteristics show different hole characteristics depending on the position of the amine.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

According to the present invention, it is possible to manufacture an organic device having excellent device characteristics of high luminance, high light emission and long lifespan, and thus there is industrial applicability.

What is claimed is:

1. A compound represented by Formula 1: 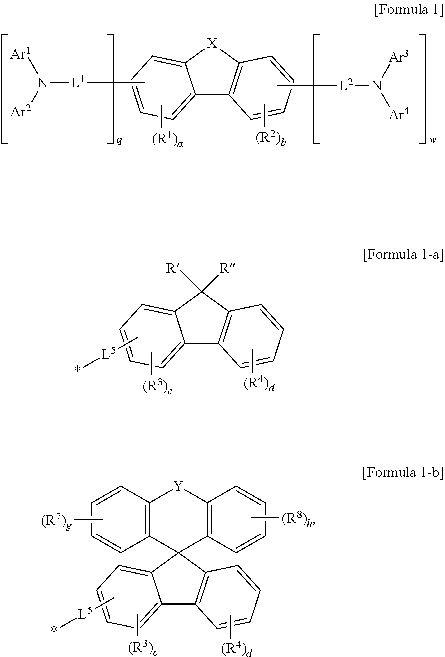
wherein:
X is O or S,
R¹ and R² are each independently selected from the group consisting of a C₆-C₆₀ aryl group; a C₂-C₆₀ heterocyclic group including at least one heteroatom of O, N, S, Si or a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxyl group; a C₆-C₆₀ aryloxy group; and where a and b are 2 or more, R¹ and R² are each in plural being the same or different,
L¹ and L² are each independently selected from the group consisting of a single bond, a C₆-C₆₀ arylene group, a C₂-C₆₀ heteroarylene group including at least one heteroatom of O, N, S, Si or and where q and w are 2 or more, L¹ and L² are each in plural being the same or different,
Ar¹, Ar², Ar³ and Ar⁴ are each independently selected from the group consisting of a C₆-C₆₀ aryl group, a substituent represented by Formula 1-a, and a substituent represented by Formula 1-b, with the proviso that at least one pair of Ar¹ and Ar², and Ar³ and Ar⁴ is a substituent represented by Formula 1-a,
a and b are each independently an integer of 0 to 4, q and w are each independently an integer of 0 to 2, with the proviso that q+w is 2 or more,
Y is NR^{a}, O or S,
R^{a} is selected from the group consisting of a C₆-C₆₀ aryl group; a C₂-C₆₀ heteroaryl group including at least one heteroatom of O, N, S, Si or F; a C₂-C₆₀ heterocyclic group including at least one heteroatom of O, N, S, Si or F; and —L′—N(R^{c})(R^{d}),
L′ is selected from the group consisting of a C₆-C₆₀ arylene group; a fluorenylene group; a fused ring group of a C₃-C₆₀ aliphatic ring and a C₆-C₆₀ aromatic ring; and a C₂-C₆₀ heterocyclic group including at least one heteroatom of O, N, S, Si or F;
R^{c} and R^{d} are each independently selected from the group consisting of a C₆-C₆₀ aryl group; a fluorenyl group; a fused ring group of a C₃-C₆₀ aliphatic ring and a C₆-C₆₀ aromatic ring; a C₂-C₆₀ heterocyclic group including at least one heteroatom of O, N, S, Si or F;
R³, R⁴, R⁷ and R⁸ are each independently selected from the group consisting of a C₆-C₆₀ aryl group; a C₂-C₆₀ heterocyclic group including at least one heteroatom of O, N, S, Si or F; a fused ring group of a C₃-C₆₀ aliphatic ring and a C₆-C₆₀ aromatic ring; a C₁-C₆₀ alkyl group; an C₂-C₆₀ alkenyl group; a C₂-C₆₀ alkynyl group; a C₁-C₆₀ alkoxyl group; and a C₆-C₆₀ aryloxy group, and where a, b, c and d are 2 or more, R³, R⁴, R⁷ and R⁸ are each in plural being the same or different,
d, g and h are each independently an integer from 0 to 4, c is an integer from 0 to 3,
L⁵ and L₅ are each independently selected from the group consisting of a single bond; a C₆-C₆₀ arylene group; a C₂-C₆₀ heteroarylene group including at least one heteroatom of O, N, S, Si or F;
R′ and R″ are independently of each other hydrogen; deuterium; halogen; cyano group; C₁-C₆₀ alkyl group; a C₂-C₆₀ alkenyl group; a C₂-C₆₀ alkynyl group; a C₁-C₆₀ alkoxyl group; a C₆-C₆₀ aryloxy group; a C₆-C₆₀ aryl group; a fluorenyl group; a C₂-C₆₀ heteroaryl group including at least one heteroatom of O, N, S, Si or F; a fused ring group of a C₃-C₆₀ aliphatic ring and a C₆-C₆₀ aromatic ring; and R′ and R″ are bonded to each other to form a spiro,
* indicates the position to be bonded,
wherein the aryl group, arylene group, heteroarylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂˜C₂₀ heterocyclic group; C₃-C₂₀ cycloalkyl group; C₇-C₂₀ arylalkyl group;
and C₈-C₂₀ arylalkenyl group; and —L′—N(R^{c})(R^{d}); wherein the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by combination thereof.

2. The compound of claim 1, wherein Formula 1 includes a compound represented by Formula 2: 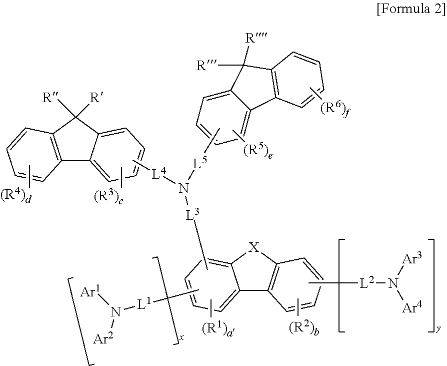
wherein:
X, R¹, R², R³, R⁴, L², L⁵, Ar¹, Ar², Ar³, Ar⁴, R′ and R″ are the same as defined in claim 1,
a′, c and e are each independently an integer of 0 to 3, b, d and f are each independently an integer of 0 to 4,
x is an integer of 0 or 1, y is an integer of 0 to 2, provided that x+y is 1 or more,
R⁵ and R⁶ are the same as the definition of R¹ to R² in claim 1,
L³ and L⁴ are the same as the definition of L² and L⁵ in claim 1,
R′″ and R″″ are the same as the definitions of R′ and R″ in claim 1.

3. The compound of claim 2, wherein Formula 2 is represented by any one of Formulas 2-1 to 2-5: 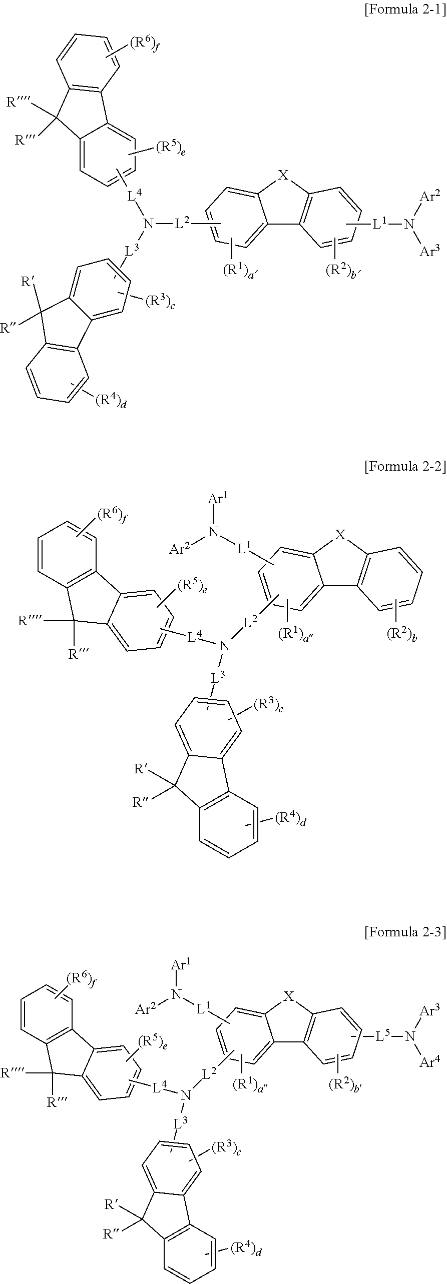 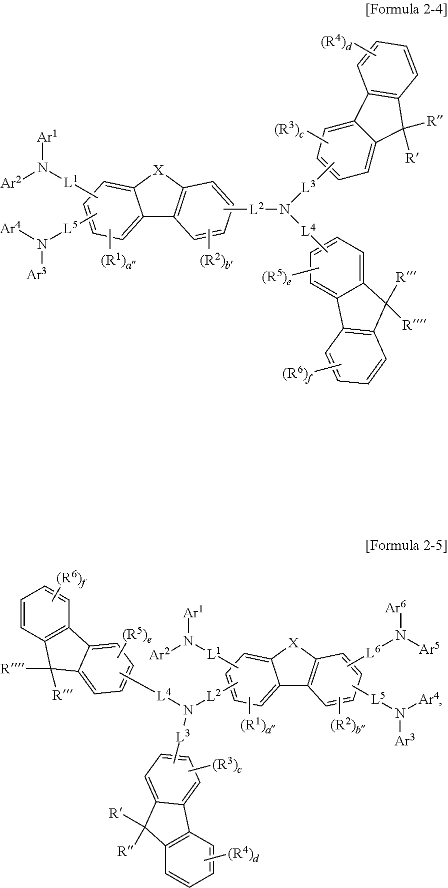
wherein:
X, R¹, R², R³, R⁴, R⁵, R⁶, L¹, L², L³, L⁴, L⁵, Ar¹, Ar², Ar³, Ar⁴, R′, R″, R′″, R″″, b, c, d, e and f are the same as defined in claim 2,
L⁶ is the same as the definition of L¹ to L⁵ in claim 2,
Ar⁵ and Ar⁶ are the same as the definitions of Ar¹ to Ar⁴ in claim 2,
a″ and b″ are independently an integer of 0 to 2, and a′ and b′ are independently an integer of 0 to 3.

4. The compound of claim 2, wherein Formula 2 is represented by Formula 2-6 or Formula 2-7: 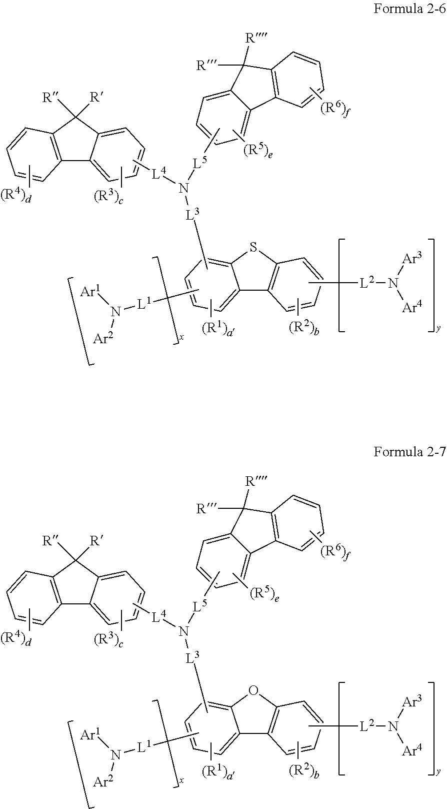
wherein R¹, R², R³, R⁴, R⁵, R⁶, L¹, L², L³, L⁴, L⁵, Ar¹, Ar², Ar³, Ar⁴, R′, R″, R′″, R″″, a′, b, c, d, e, f, x and y are the same as defined in claim 2.

5. The compound of claim 2, wherein Formula 2 is represented by any one of Formulas 2-8 to 2-11: 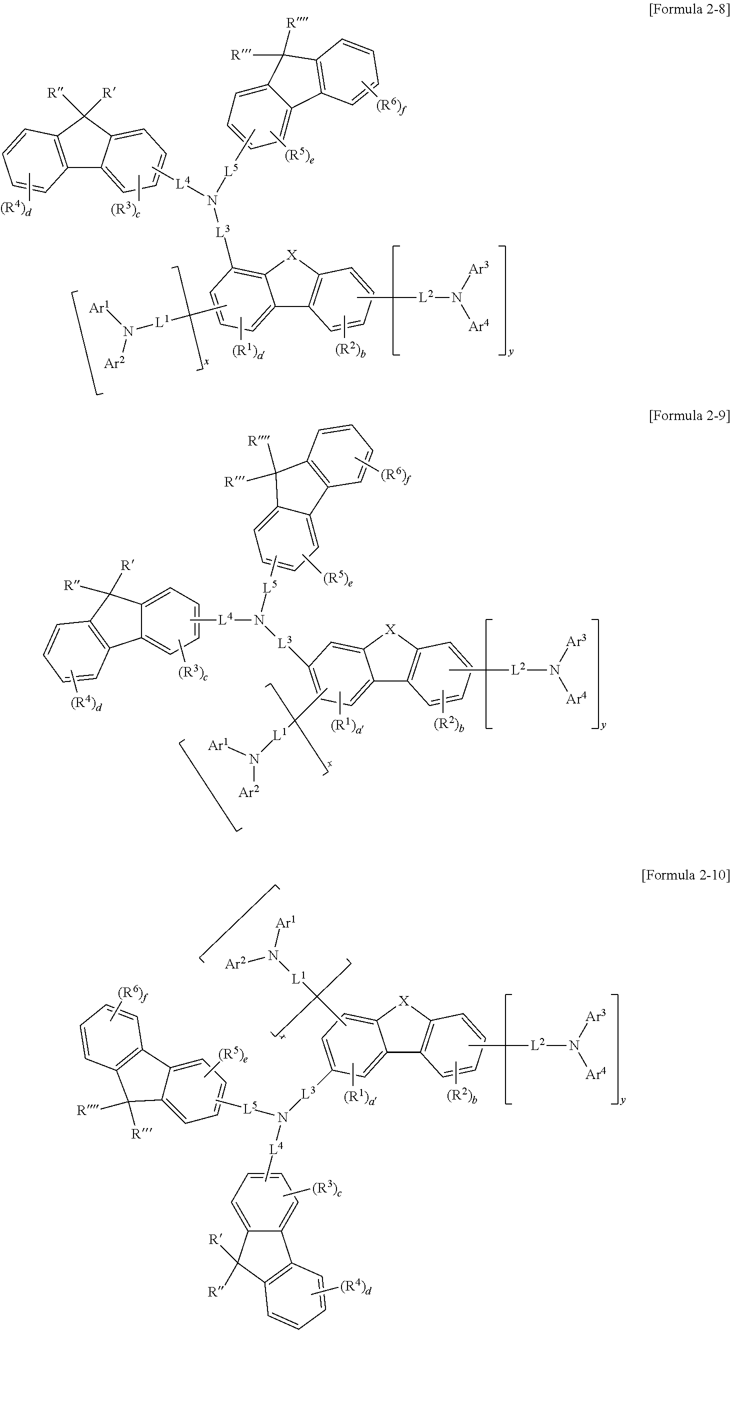 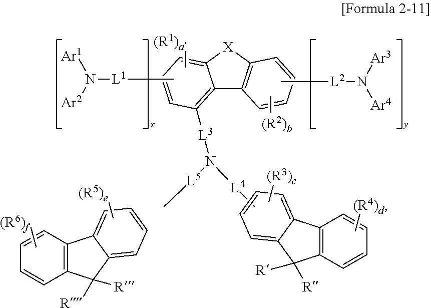
wherein X, R¹, R², R³, R⁴, R⁵, R⁶, L¹, L², L³, L⁴, L⁵, Ar¹, Ar², Ar³, Ar⁴, R′, R″, R′″, R″″, a′, b, c, d, e, f, x and y are the same as defined in claim 2.

6. The compound of claim 2, wherein Formula 2 is represented by any one of Formulas 2-12 to 2-15: 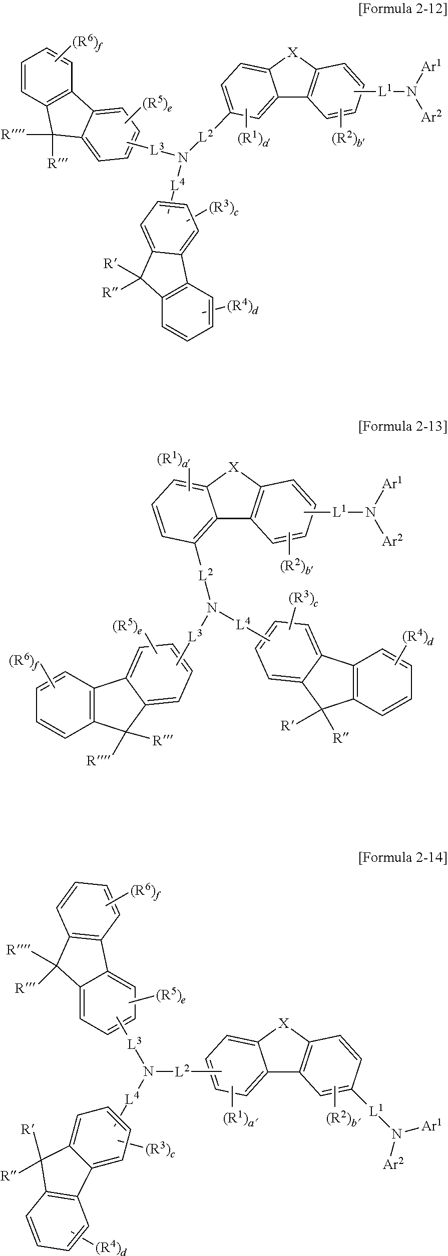 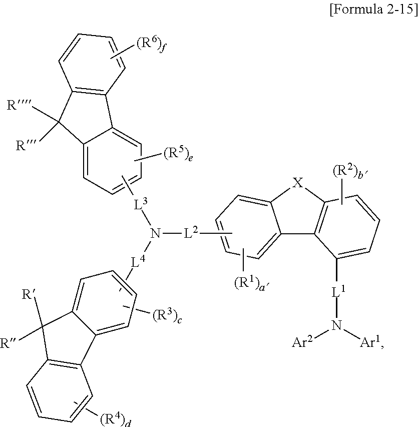
wherein:
X, R¹, R², R³, R⁴, R⁵, R⁶, L¹, L², L³, L⁴, Ar¹, Ar², R′, R″, R′″ and R″″ are the same as defined in claim 2, and
a′, b′, c and e are independently an integer of 0 to 3, and b and f are independently an integer of 0 to 4.

7. The compound of claim 2, wherein Formula 2 is represented by any one of Formulas 2-16 to 2-19: 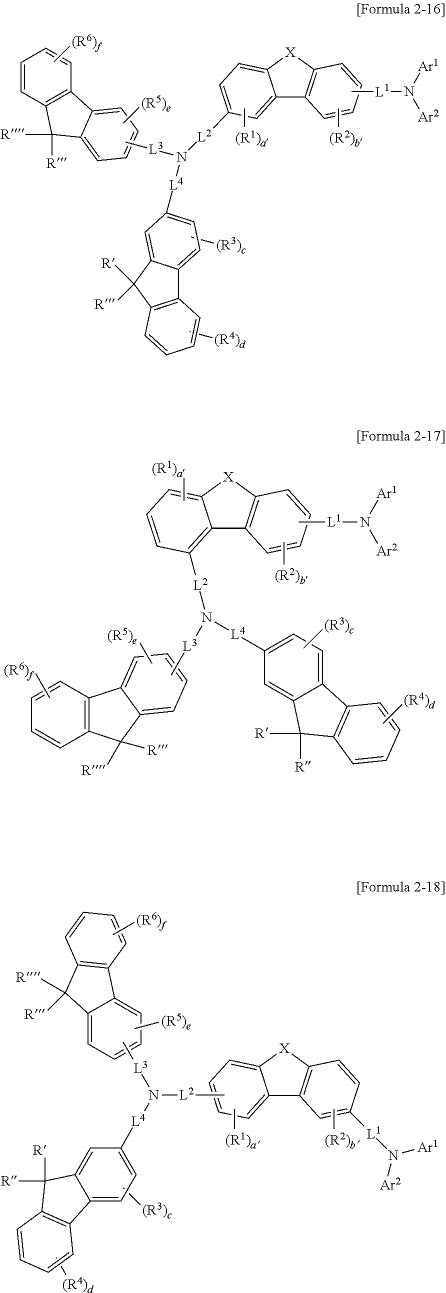 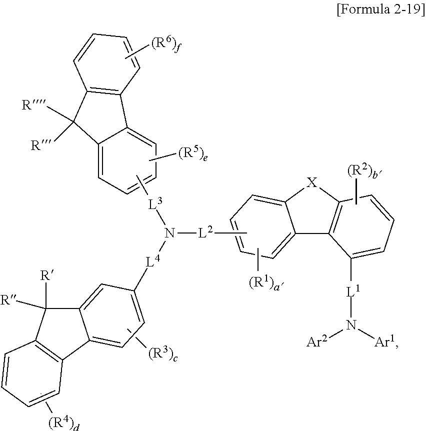
wherein:
X, R¹, R², R³, R⁴, R⁵, R⁶, L¹, L², L³, L⁴, Ar¹, Ar², R′, R″, R′″ and R″″ are the same as defined in claim 2, and
a′, b′, c and e are independently an integer of 0 to 3, and b and f are independently an integer of 0 to 4.

8. The compound of claim 2, wherein Formula 2 is represented by Formula 2-20: 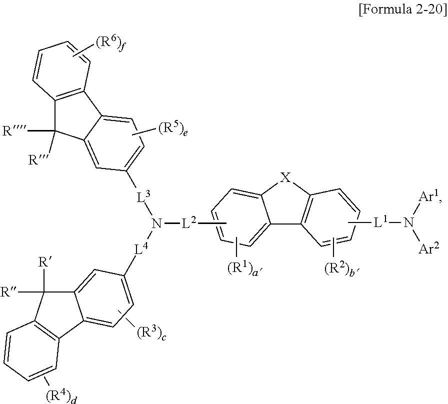
wherein:
X, R¹, R², R³, R⁴, R⁵, R⁶, L¹, L², L³, L⁴, Ar¹, Ar², R′, R″, R′″ and R″″ are the same as defined in claim 2, and
a′, b′, c and e are independently an integer of 0 to 3, and b and f are independently an integer of 0 to 4.

9. The compound of claim 2, wherein the compound represented by Formula 1 is any one of the following compounds P-1 to P-60:

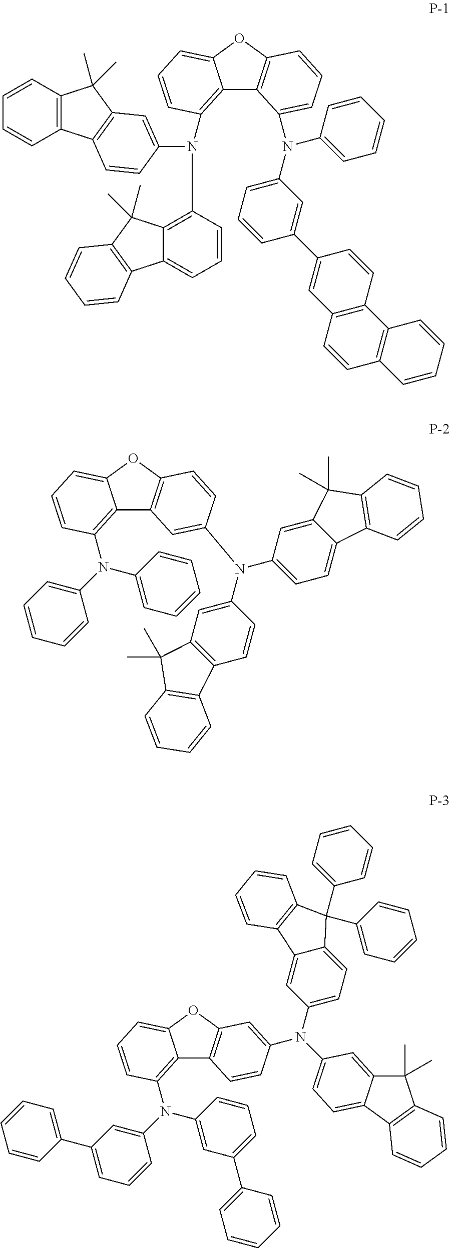 [SMILES: CC1(C)c2ccccc2-c2ccc(N(c3cccc4c3C(C)(C)c3ccccc3-4)c3cccc4oc5cccc(N(c6ccccc6)c6cccc(-c7ccc8c(ccc9ccccc98)c7)c6)c5c34)cc21 ; CC1(C)c2ccccc2-c2ccc(N(c3ccc4c(c3)C(C)(C)c3ccccc3-4)c3ccc4oc5cccc(N(c6ccccc6)c6ccccc6)c5c4c3)cc21 ; CC1(C)c2ccccc2-c2ccc(N(c3ccc4c(c3)-c3ccccc3C4(c3ccccc3)c3ccccc3)c3ccc4c(c3)oc3cccc(N(c5cccc(-c6ccccc6)c5)c5cccc(-c6ccccc6)c5)c34)cc21]

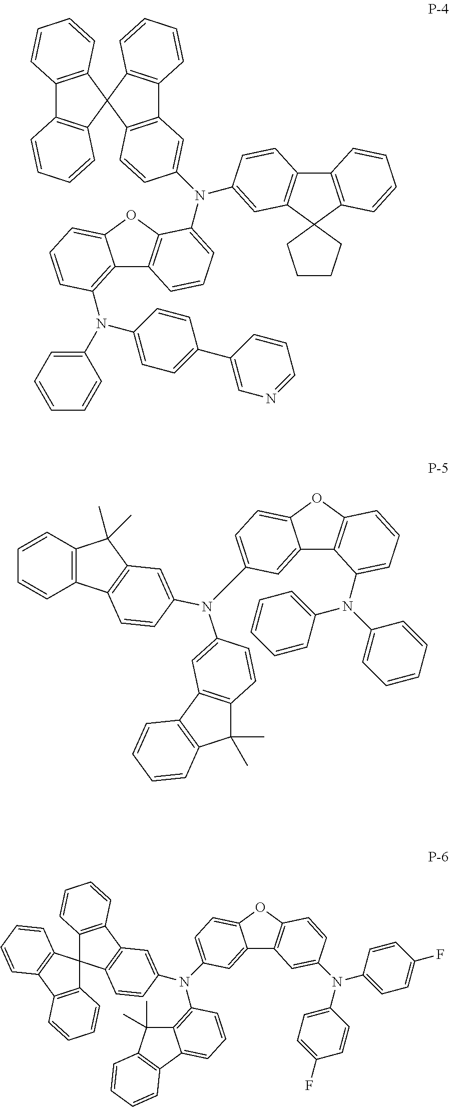 [SMILES: c1ccc(N(c2ccc(-c3cccnc3)cc2)c2cccc3oc4c(N(c5ccc6c(c5)-c5ccccc5C65c6ccccc6-c6ccccc65)c5ccc6c(c5)C5(CCCC5)c5ccccc5-6)cccc4c23)cc1 ; CC1(C)c2ccccc2-c2cc(N(c3ccc4c(c3)C(C)(C)c3ccccc3-4)c3ccc4oc5cccc(N(c6ccccc6)c6ccccc6)c5c4c3)ccc21 ; CC1(C)c2ccccc2-c2cccc(N(c3ccc4c(c3)-c3ccccc3C43c4ccccc4-c4ccccc43)c3ccc4oc5ccc(N(c6ccc(F)cc6)c6ccc(F)cc6)cc5c4c3)c21]

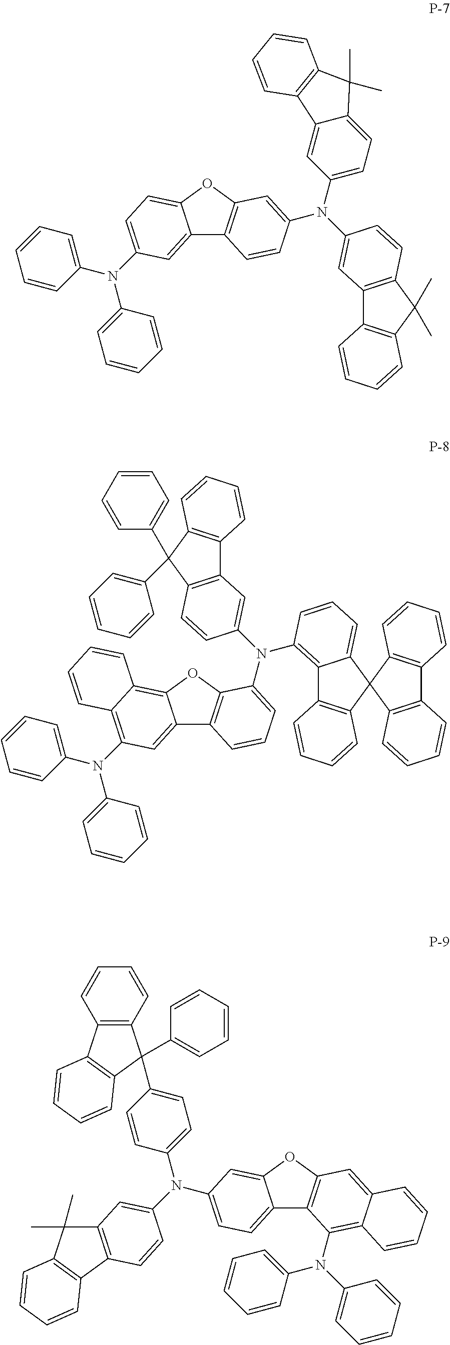 [SMILES: CC1(C)c2ccccc2-c2cc(N(c3ccc4c(c3)-c3ccccc3C4(C)C)c3ccc4c(c3)oc3ccc(N(c5ccccc5)c5ccccc5)cc34)ccc21 ; c1ccc(N(c2ccccc2)c2cc3c4cccc(N(c5ccc6c(c5)-c5ccccc5C6(c5ccccc5)c5ccccc5)c5cccc6c5-c5ccccc5C65c6ccccc6-c6ccccc65)c4oc3c3ccccc23)cc1 ; CC1(C)c2ccccc2-c2ccc(N(c3ccc(C4(c5ccccc5)c5ccccc5-c5ccccc54)cc3)c3ccc4c(c3)oc3cc5ccccc5c(N(c5ccccc5)c5ccccc5)c34)cc21]

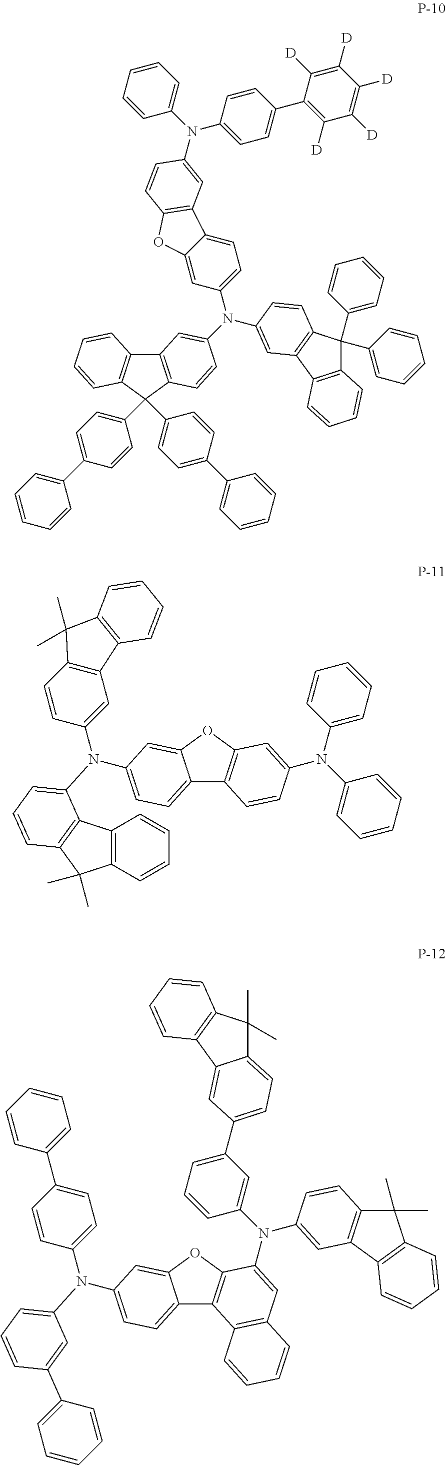 [SMILES: [2H]c1c([2H])c([2H])c(-c2ccc(N(c3ccccc3)c3ccc4oc5cc(N(c6ccc7c(c6)-c6ccccc6C7(c6ccccc6)c6ccccc6)c6ccc7c(c6)-c6ccccc6C7(c6ccc(-c7ccccc7)cc6)c6ccc(-c7ccccc7)cc6)ccc5c4c3)cc2)c([2H])c1[2H] ; CC1(C)c2ccccc2-c2cc(N(c3ccc4c(c3)oc3cc(N(c5ccccc5)c5ccccc5)ccc34)c3cccc4c3-c3ccccc3C4(C)C)ccc21 ; CC1(C)c2ccccc2-c2cc(-c3cccc(N(c4ccc5c(c4)-c4ccccc4C5(C)C)c4cc5ccccc5c5c4oc4cc(N(c6ccc(-c7ccccc7)cc6)c6cccc(-c7ccccc7)c6)ccc45)c3)ccc21]

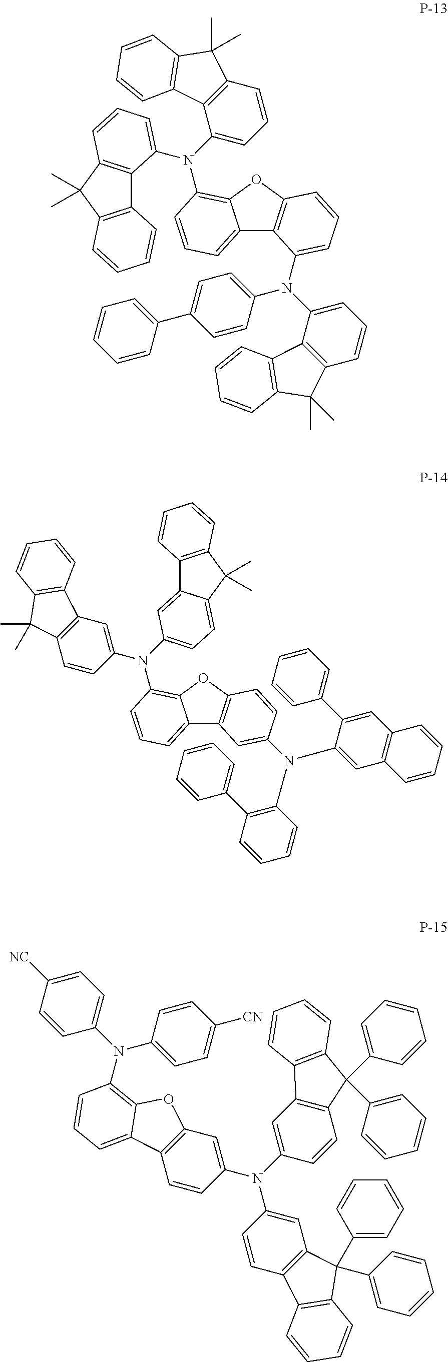 [SMILES: CC1(C)c2ccccc2-c2c(N(c3cccc4c3-c3ccccc3C4(C)C)c3cccc4c3oc3cccc(N(c5ccc(-c6ccccc6)cc5)c5cccc6c5-c5ccccc5C6(C)C)c34)cccc21 ; CC1(C)c2ccccc2-c2cc(N(c3ccc4c(c3)-c3ccccc3C4(C)C)c3cccc4c3oc3ccc(N(c5ccccc5-c5ccccc5)c5cc6ccccc6cc5-c5ccccc5)cc34)ccc21 ; N#Cc1ccc(N(c2ccc(C#N)cc2)c2cccc3c2oc2cc(N(c4ccc5c(c4)-c4ccccc4C5(c4ccccc4)c4ccccc4)c4ccc5c(c4)C(c4ccccc4)(c4ccccc4)c4ccccc4-5)ccc23)cc1]

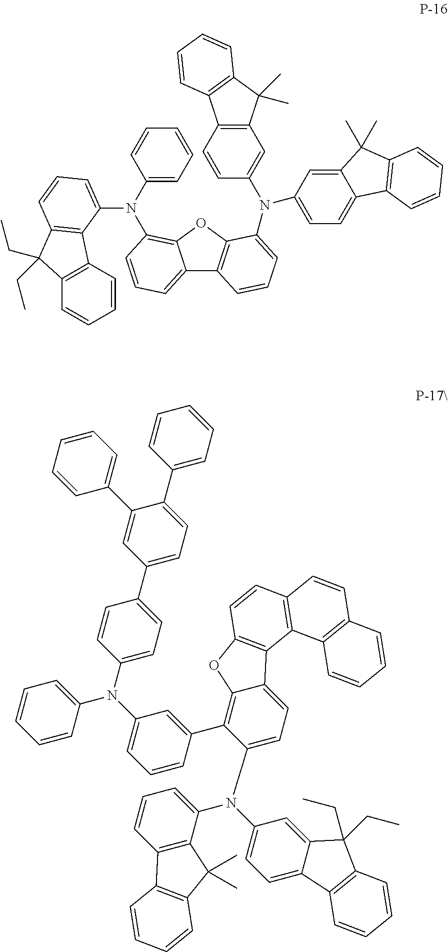 [SMILES: CCC1(CC)c2ccccc2-c2c(N(c3ccccc3)c3cccc4c3oc3c(N(c5ccc6c(c5)C(C)(C)c5ccccc5-6)c5ccc6c(c5)C(C)(C)c5ccccc5-6)cccc34)cccc21 ; CCC1(CC)c2ccccc2-c2ccc(N(c3cccc4c3C(C)(C)c3ccccc3-4)c3ccc4c(oc5ccc6ccc7ccccc7c6c54)c3-c3cccc(N(c4ccccc4)c4ccc(-c5ccc(-c6ccccc6)c(-c6ccccc6)c5)cc4)c3)cc21]

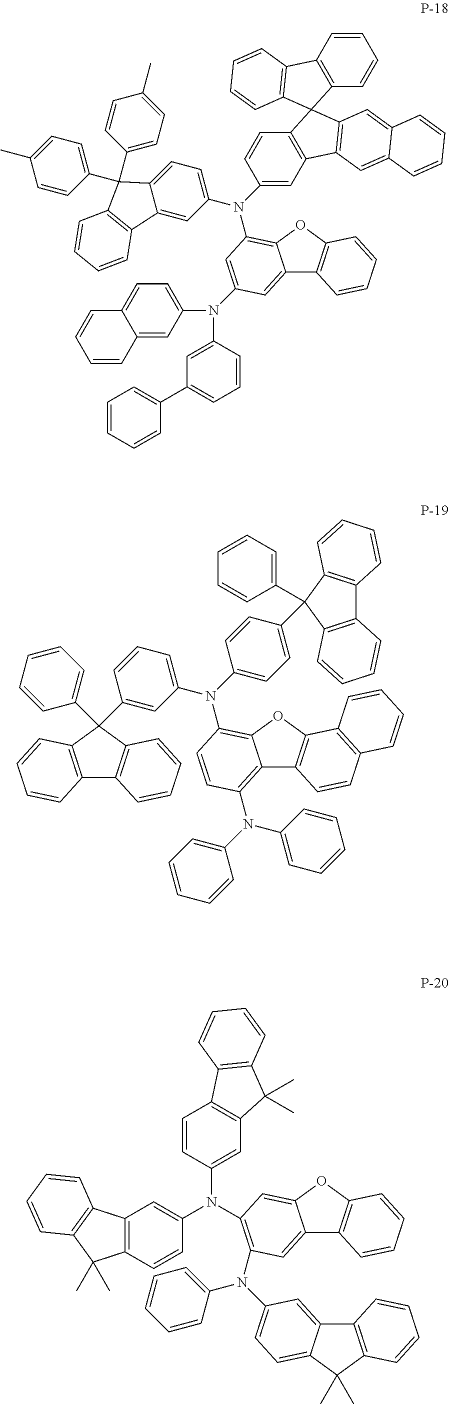 [SMILES: Cc1ccc(C2(c3ccc(C)cc3)c3ccccc3-c3cc(N(c4ccc5c(c4)-c4cc6ccccc6cc4C54c5ccccc5-c5ccccc54)c4cc(N(c5cccc(-c6ccccc6)c5)c5ccc6ccccc6c5)cc5c4oc4ccccc45)ccc32)cc1 ; c1ccc(N(c2ccccc2)c2ccc(N(c3ccc(C4(c5ccccc5)c5ccccc5-c5ccccc54)cc3)c3cccc(C4(c5ccccc5)c5ccccc5-c5ccccc54)c3)c3oc4c5ccccc5ccc4c23)cc1 ; CC1(C)c2ccccc2-c2cc(N(c3ccc4c(c3)C(C)(C)c3ccccc3-4)c3cc4oc5ccccc5c4cc3N(c3ccccc3)c3ccc4c(c3)-c3ccccc3C4(C)C)ccc21]

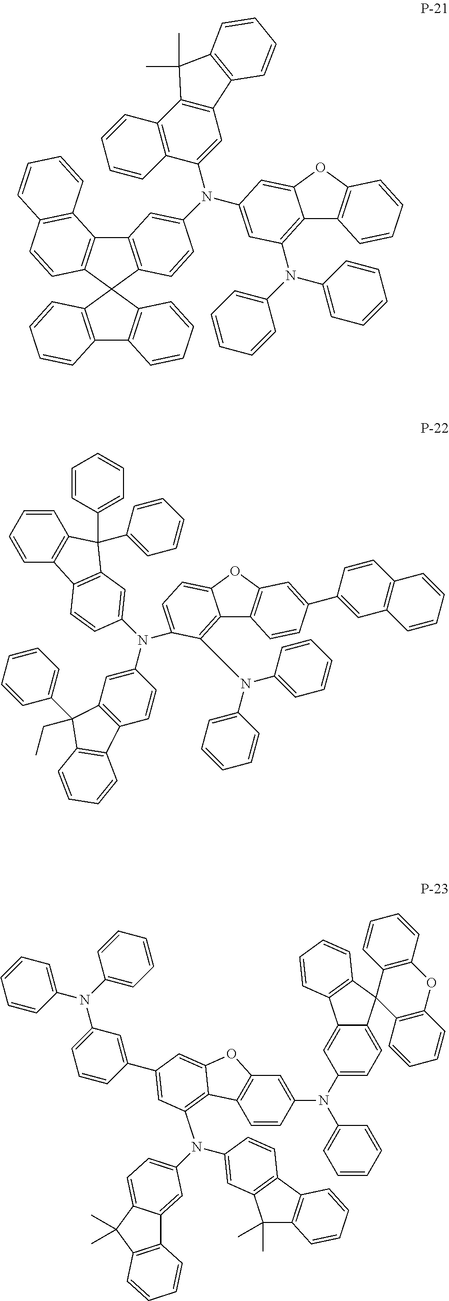 [SMILES: CC1(C)c2ccccc2-c2cc(N(c3ccc4c(c3)-c3c(ccc5ccccc35)C43c4ccccc4-c4ccccc43)c3cc(N(c4ccccc4)c4ccccc4)c4c(c3)oc3ccccc34)c3ccccc3c21 ; CCC1(c2ccccc2)c2ccccc2-c2ccc(N(c3ccc4c(c3)C(c3ccccc3)(c3ccccc3)c3ccccc3-4)c3ccc4oc5cc(-c6ccc7ccccc7c6)ccc5c4c3N(c3ccccc3)c3ccccc3)cc21 ; CC1(C)c2ccccc2-c2cc(N(c3ccc4c(c3)C(C)(C)c3ccccc3-4)c3cc(-c4cccc(N(c5ccccc5)c5ccccc5)c4)cc4oc5cc(N(c6ccccc6)c6ccc7c(c6)-c6ccccc6C76c7ccccc7Oc7ccccc76)ccc5c34)ccc21]

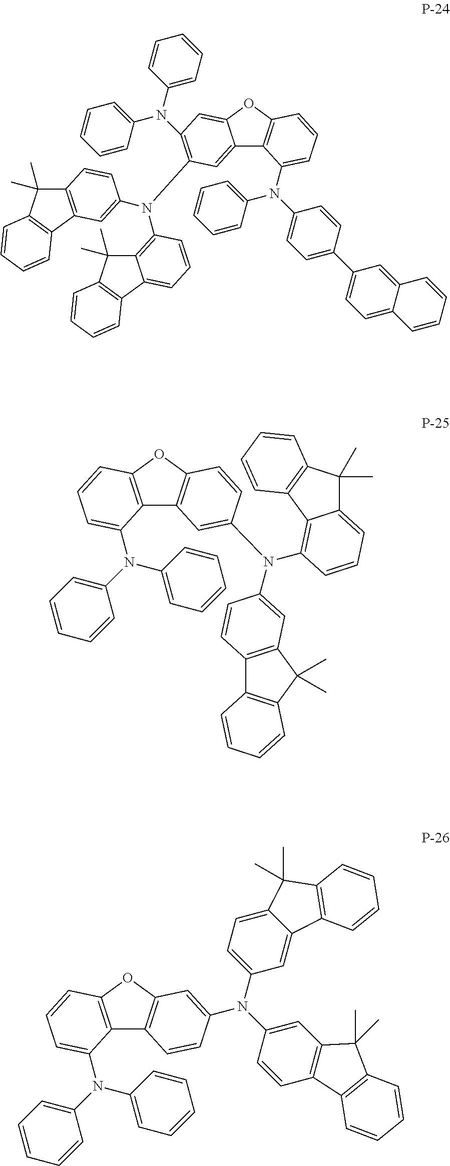 [SMILES: CC1(C)c2ccccc2-c2cc(N(c3cc4c(cc3N(c3ccccc3)c3ccccc3)oc3cccc(N(c5ccccc5)c5ccc(-c6ccc7ccccc7c6)cc5)c34)c3cccc4c3C(C)(C)c3ccccc3-4)ccc21 ; CC1(C)c2ccccc2-c2ccc(N(c3ccc4oc5cccc(N(c6ccccc6)c6ccccc6)c5c4c3)c3cccc4c3-c3ccccc3C4(C)C)cc21 ; CC1(C)c2ccccc2-c2cc(N(c3ccc4c(c3)C(C)(C)c3ccccc3-4)c3ccc4c(c3)oc3cccc(N(c5ccccc5)c5ccccc5)c34)ccc21]

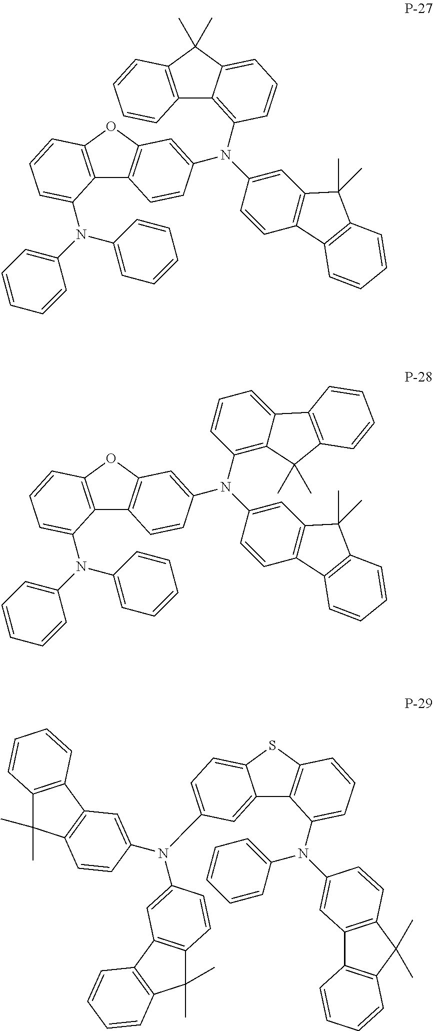

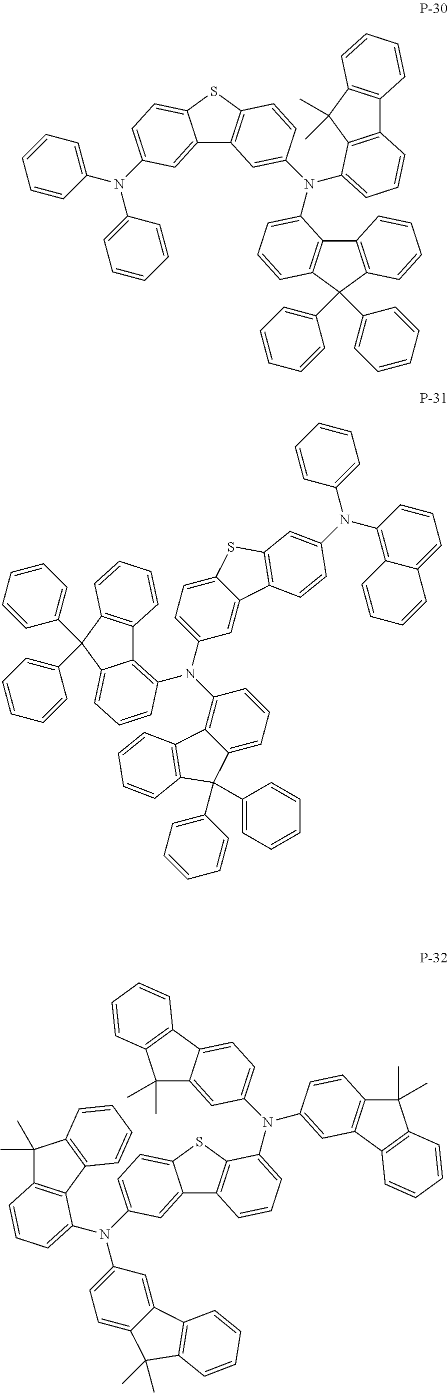 [SMILES: CC1(C)c2ccccc2-c2cccc(N(c3ccc4sc5ccc(N(c6ccccc6)c6ccccc6)cc5c4c3)c3cccc4c3-c3ccccc3C4(c3ccccc3)c3ccccc3)c21 ; c1ccc(N(c2ccc3c(c2)sc2ccc(N(c4cccc5c4-c4ccccc4C5(c4ccccc4)c4ccccc4)c4cccc5c4-c4ccccc4C5(c4ccccc4)c4ccccc4)cc23)c2cccc3ccccc23)cc1 ; CC1(C)c2ccccc2-c2cc(N(c3ccc4sc5c(N(c6ccc7c(c6)-c6ccccc6C7(C)C)c6ccc7c(c6)C(C)(C)c6ccccc6-7)cccc5c4c3)c3cccc4c3-c3ccccc3C4(C)C)ccc21]

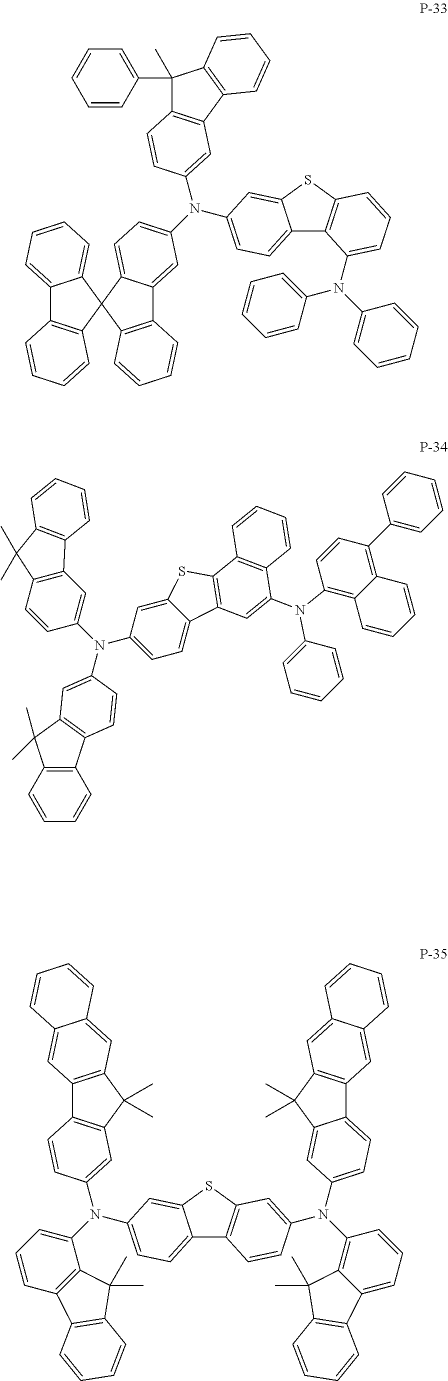 [SMILES: CC1(c2ccccc2)c2ccccc2-c2cc(N(c3ccc4c(c3)-c3ccccc3C43c4ccccc4-c4ccccc43)c3ccc4c(c3)sc3cccc(N(c5ccccc5)c5ccccc5)c34)ccc21 ; CC1(C)c2ccccc2-c2cc(N(c3ccc4c(c3)C(C)(C)c3ccccc3-4)c3ccc4c(c3)sc3c5ccccc5c(N(c5ccccc5)c5ccc(-c6ccccc6)c6ccccc56)cc43)ccc21 ; CC1(C)c2cc(N(c3ccc4c(c3)sc3cc(N(c5ccc6c(c5)C(C)(C)c5cc7ccccc7cc5-6)c5cccc6c5C(C)(C)c5ccccc5-6)ccc34)c3cccc4c3C(C)(C)c3ccccc3-4)ccc2-c2cc3ccccc3cc21]

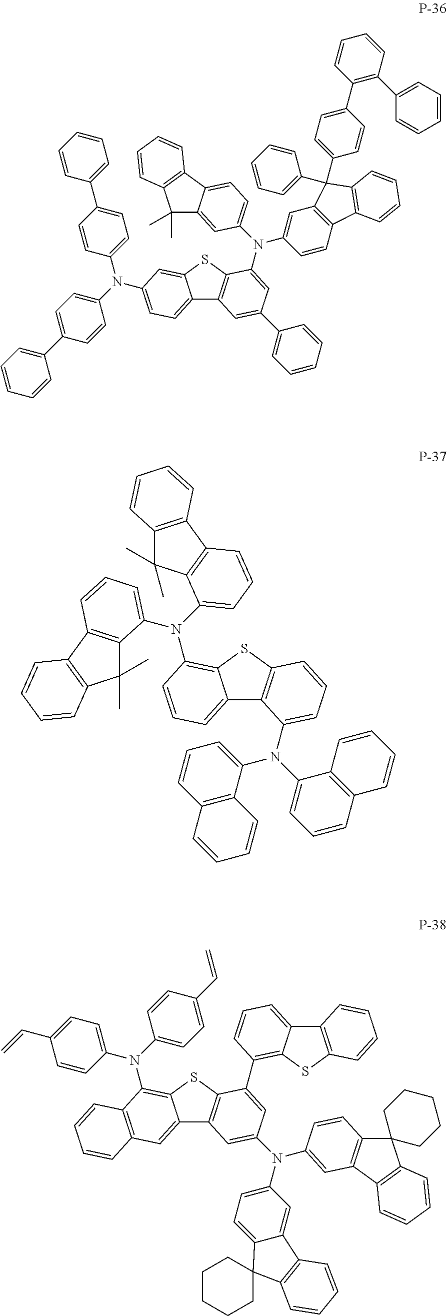 [SMILES: CC1(C)c2ccccc2-c2ccc(N(c3ccc4c(c3)C(c3ccccc3)(c3ccc(-c5ccccc5-c5ccccc5)cc3)c3ccccc3-4)c3cc(-c4ccccc4)cc4c3sc3cc(N(c5ccc(-c6ccccc6)cc5)c5ccc(-c6ccccc6)cc5)ccc34)cc21 ; CC1(C)c2ccccc2-c2cccc(N(c3cccc4c3C(C)(C)c3ccccc3-4)c3cccc4c3sc3cccc(N(c5cccc6ccccc56)c5cccc6ccccc56)c34)c21 ; C=Cc1ccc(N(c2ccc(C=C)cc2)c2c3ccccc3cc3c2sc2c(-c4cccc5c4sc4ccccc45)cc(N(c4ccc5c(c4)-c4ccccc4C54CCCCC4)c4ccc5c(c4)-c4ccccc4C54CCCCC4)cc23)cc1]

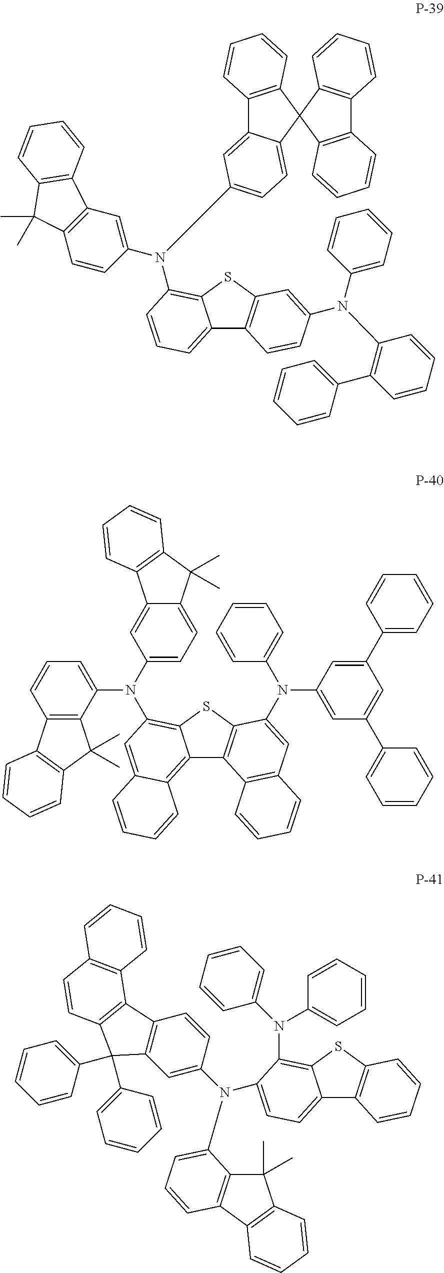 [SMILES: CC1(C)c2ccccc2-c2cc(N(c3ccc4c(c3)-c3ccccc3C43c4ccccc4-c4ccccc43)c3cccc4c3sc3cc(N(c5ccccc5)c5ccccc5-c5ccccc5)ccc34)ccc21 ; CC1(C)c2ccccc2-c2cc(N(c3cccc4c3C(C)(C)c3ccccc3-4)c3cc4ccccc4c4c3sc3c(N(c5ccccc5)c5cc(-c6ccccc6)cc(-c6ccccc6)c5)cc5ccccc5c34)ccc21 ; CC1(C)c2ccccc2-c2cccc(N(c3ccc4c(c3)C(c3ccccc3)(c3ccccc3)c3ccc5ccccc5c3-4)c3ccc4c(sc5ccccc54)c3N(c3ccccc3)c3ccccc3)c21]

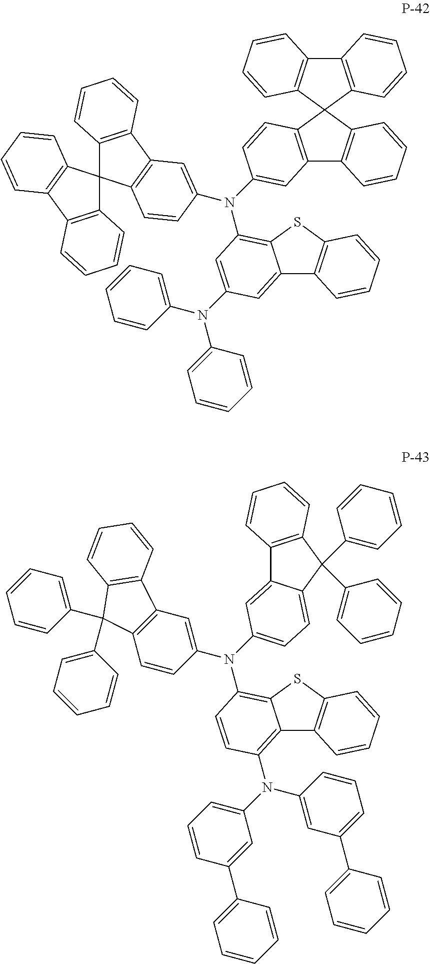 [SMILES: c1ccc(N(c2ccccc2)c2cc(N(c3ccc4c(c3)-c3ccccc3C43c4ccccc4-c4ccccc43)c3ccc4c(c3)-c3ccccc3C43c4ccccc4-c4ccccc43)c3sc4ccccc4c3c2)cc1 ; c1ccc(-c2cccc(N(c3cccc(-c4ccccc4)c3)c3ccc(N(c4ccc5c(c4)-c4ccccc4C5(c4ccccc4)c4ccccc4)c4ccc5c(c4)-c4ccccc4C5(c4ccccc4)c4ccccc4)c4sc5ccccc5c34)c2)cc1]

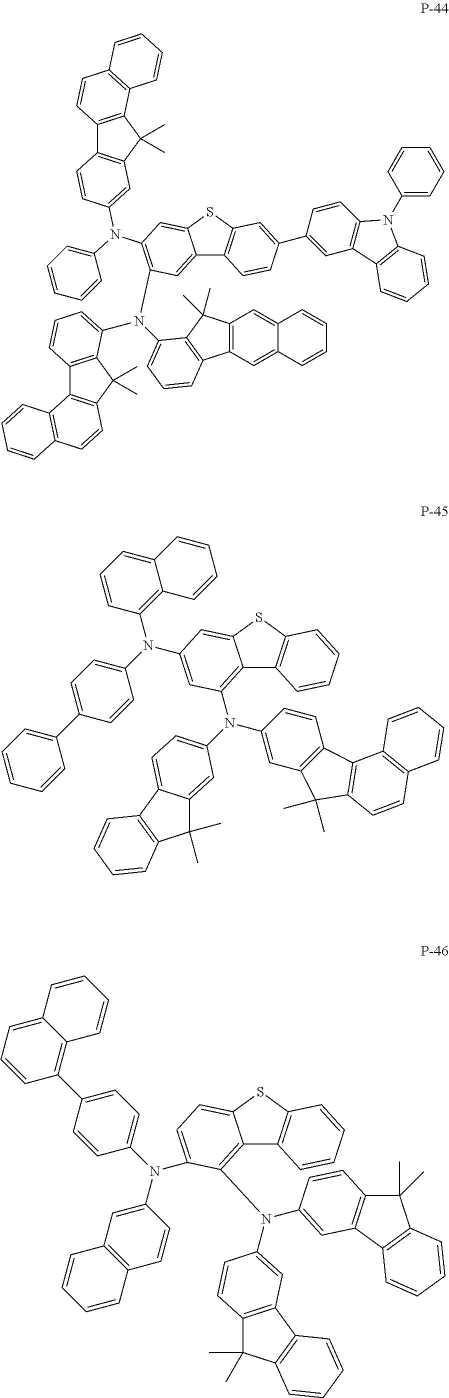 [SMILES: CC1(C)c2cc3ccccc3cc2-c2cccc(N(c3cc4c(cc3N(c3ccccc3)c3ccc5c(c3)C(C)(C)c3c-5ccc5ccccc35)sc3cc(-c5ccc6c(c5)c5ccccc5n6-c5ccccc5)ccc34)c3cccc4c3C(C)(C)c3ccc5ccccc5c3-4)c21 ; CC1(C)c2ccccc2-c2ccc(N(c3ccc4c(c3)C(C)(C)c3ccc5ccccc5c3-4)c3cc(N(c4ccc(-c5ccccc5)cc4)c4cccc5ccccc45)cc4sc5ccccc5c34)cc21 ; CC1(C)c2ccccc2-c2cc(N(c3ccc4c(c3)-c3ccccc3C4(C)C)c3c(N(c4ccc(-c5cccc6ccccc56)cc4)c4ccc5ccccc5c4)ccc4sc5ccccc5c34)ccc21]

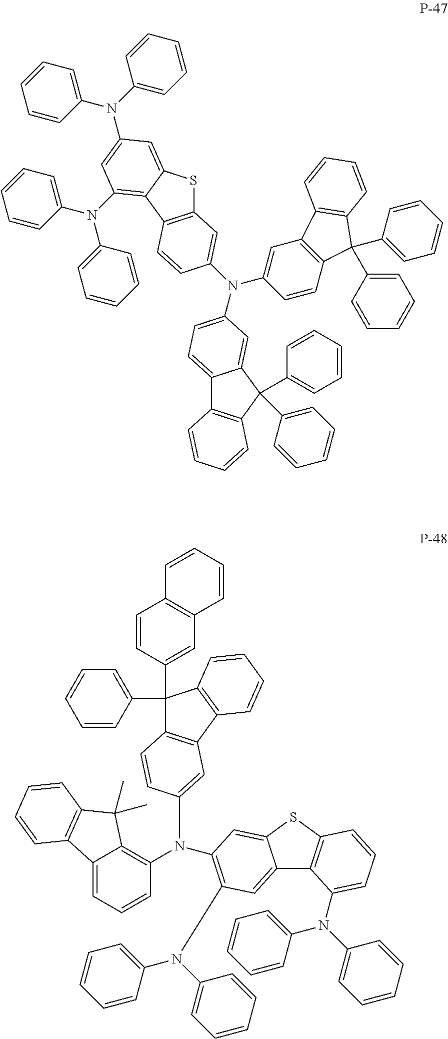 [SMILES: c1ccc(N(c2ccccc2)c2cc(N(c3ccccc3)c3ccccc3)c3c(c2)sc2cc(N(c4ccc5c(c4)-c4ccccc4C5(c4ccccc4)c4ccccc4)c4ccc5c(c4)C(c4ccccc4)(c4ccccc4)c4ccccc4-5)ccc23)cc1 ; CC1(C)c2ccccc2-c2cccc(N(c3ccc4c(c3)-c3ccccc3C4(c3ccccc3)c3ccc4ccccc4c3)c3cc4sc5cccc(N(c6ccccc6)c6ccccc6)c5c4cc3N(c3ccccc3)c3ccccc3)c21]

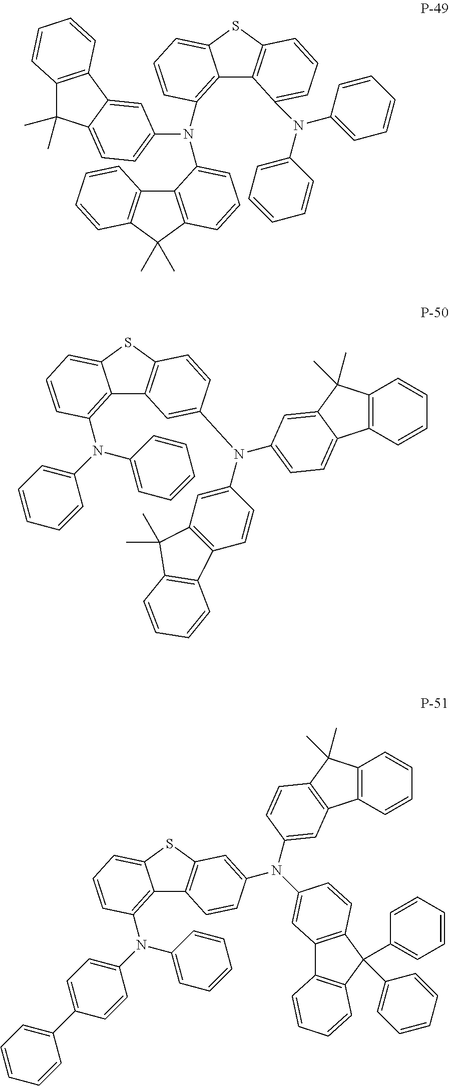 [SMILES: CC1(C)c2ccccc2-c2cc(N(c3cccc4c3-c3ccccc3C4(C)C)c3cccc4sc5cccc(N(c6ccccc6)c6ccccc6)c5c34)ccc21 ; CC1(C)c2ccccc2-c2ccc(N(c3ccc4c(c3)C(C)(C)c3ccccc3-4)c3ccc4sc5cccc(N(c6ccccc6)c6ccccc6)c5c4c3)cc21 ; CC1(C)c2ccccc2-c2cc(N(c3ccc4c(c3)-c3ccccc3C4(c3ccccc3)c3ccccc3)c3ccc4c(c3)sc3cccc(N(c5ccccc5)c5ccc(-c6ccccc6)cc5)c34)ccc21]

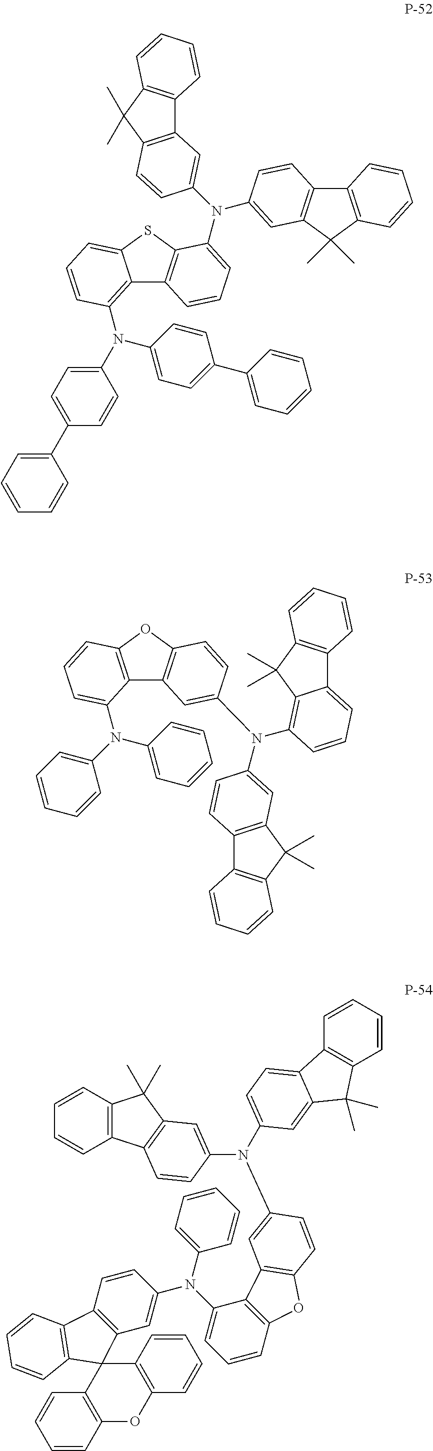 [SMILES: CC1(C)c2ccccc2-c2cc(N(c3ccc4c(c3)C(C)(C)c3ccccc3-4)c3cccc4c3sc3cccc(N(c5ccc(-c6ccccc6)cc5)c5ccc(-c6ccccc6)cc5)c34)ccc21 ; CC1(C)c2ccccc2-c2ccc(N(c3ccc4oc5cccc(N(c6ccccc6)c6ccccc6)c5c4c3)c3cccc4c3C(C)(C)c3ccccc3-4)cc21 ; CC1(C)c2ccccc2-c2ccc(N(c3ccc4c(c3)C(C)(C)c3ccccc3-4)c3ccc4oc5cccc(N(c6ccccc6)c6ccc7c(c6)C6(c8ccccc8Oc8ccccc86)c6ccccc6-7)c5c4c3)cc21]

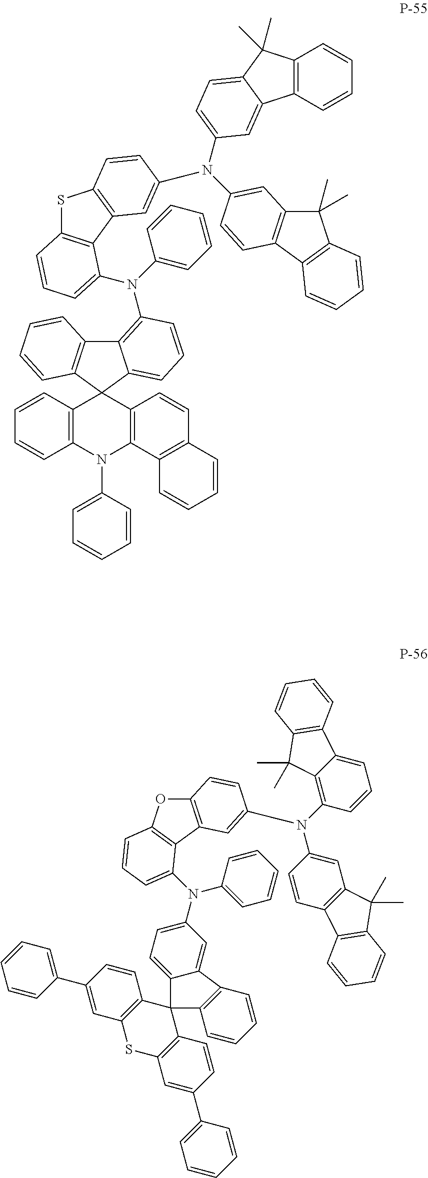 [SMILES: CC1(C)c2ccccc2-c2cc(N(c3ccc4c(c3)C(C)(C)c3ccccc3-4)c3ccc4sc5cccc(N(c6ccccc6)c6cccc7c6-c6ccccc6C76c7ccccc7N(c7ccccc7)c7c6ccc6ccccc76)c5c4c3)ccc21 ; CC1(C)c2ccccc2-c2ccc(N(c3ccc4oc5cccc(N(c6ccccc6)c6ccc7c(c6)-c6ccccc6C76c7ccc(-c8ccccc8)cc7Sc7cc(-c8ccccc8)ccc76)c5c4c3)c3cccc4c3C(C)(C)c3ccccc3-4)cc21]

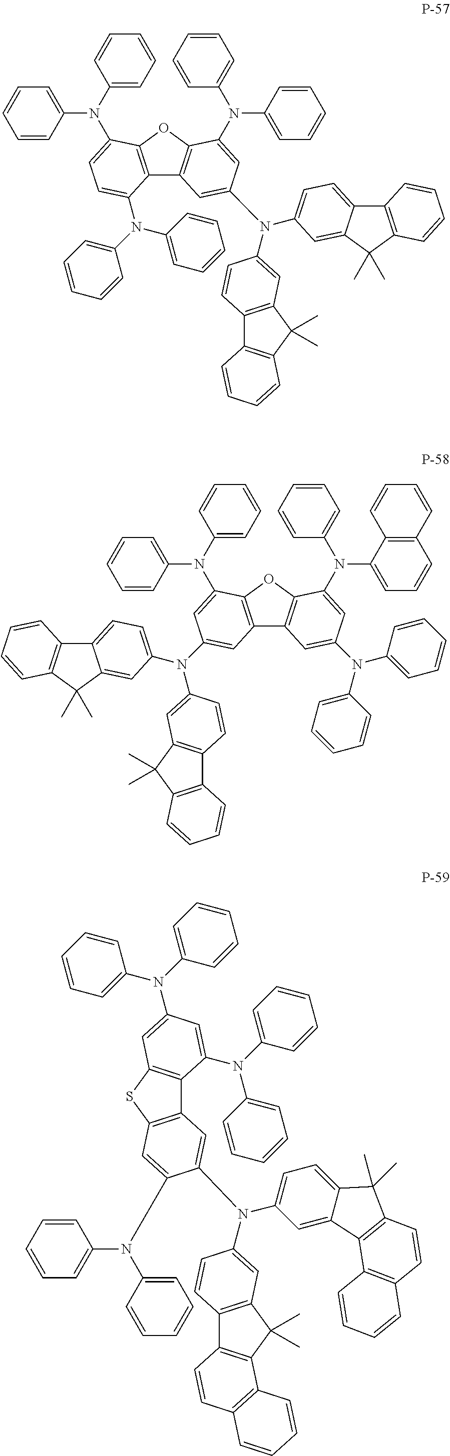 [SMILES: CC1(C)c2ccccc2-c2ccc(N(c3ccc4c(c3)C(C)(C)c3ccccc3-4)c3cc(N(c4ccccc4)c4ccccc4)c4oc5c(N(c6ccccc6)c6ccccc6)ccc(N(c6ccccc6)c6ccccc6)c5c4c3)cc21 ; CC1(C)c2ccccc2-c2ccc(N(c3ccc4c(c3)C(C)(C)c3ccccc3-4)c3cc(N(c4ccccc4)c4ccccc4)c4oc5c(N(c6ccccc6)c6cccc7ccccc67)cc(N(c6ccccc6)c6ccccc6)cc5c4c3)cc21 ; CC1(C)c2ccc(N(c3ccc4c(c3)C(C)(C)c3c-4ccc4ccccc34)c3cc4c(cc3N(c3ccccc3)c3ccccc3)sc3cc(N(c5ccccc5)c5ccccc5)cc(N(c5ccccc5)c5ccccc5)c34)cc2-c2c1ccc1ccccc21]

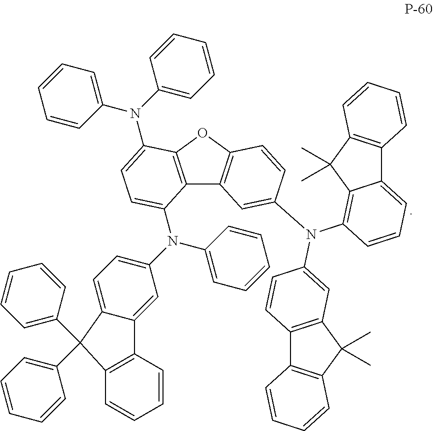 [SMILES: CC1(C)c2ccccc2-c2ccc(N(c3ccc4oc5c(N(c6ccccc6)c6ccccc6)ccc(N(c6ccccc6)c6ccc7c(c6)-c6ccccc6C7(c6ccccc6)c6ccccc6)c5c4c3)c3cccc4c3C(C)(C)c3ccccc3-4)cc21]

10. An organic electronic element comprising an anode, a cathode, and an organic material layer formed between the anode and the cathode, wherein the organic material layer comprises a single compound or 2 or more compounds represented by Formula 1 of claim 1.

11. The organic electronic element of claim 10, wherein the organic material layer comprises at least one of a hole injection layer, a hole transport layer, an emitting auxiliary layer, an emitting layer, an electron transport auxiliary layer, an electron transport layer, and an electron injection layer.

12. The organic electronic element of claim 10, wherein the organic material layer is an emitting auxiliary layer.

13. The organic electronic element of claim 10, wherein the organic electronic element further comprises a light efficiency enhancing layer formed on at least one surface of the anode and the cathode, the surface being opposite to the organic material layer.

14. The organic electronic element of claim 10, wherein the organic material layer comprises 2 or more stacks including a hole transport layer, an emitting layer, and an electron transport layer sequentially formed on the anode.

15. The organic electronic element of claim 10, wherein the organic material layer further comprises a charge generation layer formed between the 2 or more stacks.

16. The organic electronic element of claim 10, wherein the organic electronic element is a green organic electronic element.

17. The organic electronic element of claim 16, wherein the organic material layer is an emitting auxiliary layer of the green organic electronic element.

18. An electronic device comprising: a display device comprising the organic electronic element of claim 10; and a control unit for driving the display device.

19. An electronic device according to claim 18, wherein the organic electronic element is at least one of an OLED, an organic solar cell, an organic photo conductor (OPC), an organic transistor (organic TFT), and an element for monochromic or white illumination.
